(19) United States
(12) Patent Application Publication
(10) Pub. No.: US 2020/0030412 A1
(43) Pub. Date: Jan. 30, 2020

(54) IL-12 COMPOSITIONS AND METHODS OF USE IN HEMATOPOIETIC RECOVERY

(71) Applicant: NEUMEDICINES INC., Pasadena, CA (US)

(72) Inventor: Lena A. BASILE, Tujunga, CA (US)

(21) Appl. No.: 16/291,999

(22) Filed: Mar. 4, 2019

Related U.S. Application Data
(63) Continuation of application No. 14/928,439, filed on Oct. 30, 2015, now abandoned.
(60) Provisional application No. 62/073,197, filed on Oct. 31, 2014.

(51) Int. Cl. A61K 38/20

(52) U.S. Cl. CPC A61K 38/208

(57) ABSTRACT

Aspects and embodiments of the instant disclosure provide therapeutic methods and compositions comprising interleukin 12 (IL-12) useful for improving hematopoietic recovery HSCT transplantation in a subject. In particular, the instant disclosure provide exemplary methods and compositions comprising IL-12 promoted hematopoiesis and increased the recovery of peripheral blood cells and survival in lethally irradiated mice as effectively as a BMCT, indicating that rHuIL-12 therapy can to increase HSC engraftment following HSCT. We identified IL-12Rβ2 expressing cells in irradiated mouse bone marrow which are potential targets of IL-12. Administration of rMuIL-12 increased the number of IL-12R□2 expressing Lin− cells in mouse bone marrow, indicating that bone marrow HSCs and niche cells are the direct target of rMuIL-12 and that hematopoiesis-promoting activity of rMuIL-12 is mediated by IL-12 receptors on HSCs. Finally, we show expression of IL-12β2 on human bone marrow lin− and CD34+ cells, indicating a potential role for IL-12 in human transplantation.

### CROSS-REFERENCE TO RELATED APPLICATIONS

This application claims the benefit of priority from U.S. Provisional Patent Application No. 62/073,197, filed Oct. 31, 2014. The entire contents of which are incorporated herein by reference in its entirety.

### FIELD

The present disclosure relates generally to novel methods and compositions for transplantation. In particular, methods and compositions for promoting hematopoietic recovery following stem cell transplantation comprising administering to a subject in need thereof a therapeutically effective amount of a pharmaceutical composition comprising IL-12.

### BACKGROUND

The following includes information that may be useful in understanding various aspects and embodiments of the present disclosure. It is not an admission that any of the information provided herein is prior art, or relevant, to the presently described or claimed inventions, or that any publication or document that is specifically or implicitly referenced is prior art.

Stem cell transplantation is a medical procedure in the fields of hematology and oncology, which may be performed for people with diseases of the blood, bone marrow, or certain cancers. Hematopoietic stem cell transplantation remains a risky procedure with many possible complications, and has traditionally been reserved for patients with life-threatening diseases. While occasionally used experimentally in nonmalignant and non-hematologic indications such as severe disabling auto-immune disease and cardiovascular disease, the risk of fatal complications appears too high to gain wider acceptance.

A total of 50,417 first hematopoietic stem cell transplants were reported as taking place worldwide in 2006, according to a global survey of 1327 centers in 71 countries conducted by the Worldwide Network for Blood and Marrow Transplantation. Of these, 28,901 (57%) were autologous and 21,516 (43%) were allogeneic (11,928 from family donors and 9,588 from unrelated donors). The main indications for transplant were lymphoproliferative disorders (54.5%) and leukemias (33.8%), and the majority took place in either Europe (48%) or the Americas (36%). In 2009, according to the world marrow donor association, stem cell products provided for unrelated transplantation worldwide had increased to 15,399 (3,445 bone marrow donations, 8,162 peripheral blood stem cell donations, and 3,792 cord blood units).

In the past, the hematopoietic stem cells were harvested from the bone marrow directly; however, currently these stem cells can be directly collected from the blood stream of patients after they have been given a growth factor which causes the stem cells to move from the marrow into the circulation. The instrument used to harvest the stem cells is called an apheresis machine. This type of transplant is called an autologous transplant as the stem cells are actually collected from the patient before the high dose therapy is given. The other major form of transplant is referred to an allogeneic transplant where the hematopoietic stem cells are collected from a donor (usually a brother or sister or matched donor). An allogeneic transplant has an added benefit as the patient is essentially getting a new immune system. Scientists have now recognized that it is this new immune system which is often able to eradicate tumor cells which remain even after patients receive high dose therapy. This phenomenon is known as graft versus tumor (GVT) effect.

Although widely used, hematopoietic cellular transplantation, whether autologous or allogenic, remains a high risk procedure. Thus the field of hematopoietic cellular transplantation has undergone changes over the last five to ten years. In particular, a phenomenon was noted where patients who had relapsed after an allogeneic transplant, subsequently were able to be placed back into a complete remission and ultimately cured of their disease when immune effector cells (T-lymphocytes) from the donor were re-infused into the patient. This information lead to a paradigm shift in the transplant field and hence lead to the birth of the non-myeloablative allogeneic stem cell transplant which is also known by other names such as: “mini-allo” transplant, “transplant lite”, “drive-thru” transplant, “reduced intensity” transplant, or “mixed chimera” transplant.

But even with the introduction of mini-transplants and new procedures for hematopoietic cellular transplantation, the risk of infection and other complications remains high. Major complications are veno-occlusive disease, mucositis, infections (sepsis), graft versus-host disease and the development of new malignancies. Thus, novel agents that can assist in improving the outcome for patients undergoing hematopoietic cellular transplantation procedures are desired. Such novel agents would increase the chance for hematopoietic recovery, while reducing the chance for the serious complications following hematopoietic cellular transplantation.

### SUMMARY OF THE INVENTION

Accordingly, there is a need for novel methods and compositions useful for hematopoietic recovery following hematopoietic stem cell transplantation (HSCT).

The present disclosure provides methods and therapeutic agents that target multiple pathways of hematopoiesis and innate immunity and can be used therapeutically for a broad range of clinical disorders including hematopoietic recovery following hematopoietic stem cell transplantation (HSCT). In some aspects the present disclosure provides methods and therapeutic agents that can improve hematopoietic recovery following HSCT.

In one aspect, the invention relates to composition comprising a recombinant human interleukin-12 (rHuIL-12) and/or its mouse homologue, IL-12 (rMuIL-12), and methods of using those compositions to reconstitute bone marrow using a single, low dose administered either before or after total body irradiation (TBI). It has been surprisingly shown, for example, that recombinant human interleukin-12 (rHuIL-12) and its mouse homologue, IL-12 (rMuIL-12) have the remarkable ability to reconstitute bone marrow using a single, low dose administered either before or after total body irradiation (TBI).

In other aspects the invention relates to methods of administering rHuIL-12 to stimulate hematopoiesis, which can act, for example, through interaction of rHuIL-12 with IL-12 receptors expressed on HSC and niche cells. In other aspects, the invention relates to methods of treating with rHuIL-12 as an adjunct to hematopoietic cellular transplants or other methods for enhancing HSC engraftment and bone marrow recovery following transplantation.

In one aspect, a method of protecting a subject from system, organ, tissue, or cellular damage, following exposure of the subject to ionizing radiation comprising: administering a dose of therapeutically effective amount of a pharmaceutical composition comprising substantially isolated IL-12 to the subject following myoablation and non-myoablation. Exemplary myeloablative methods can include for example, radiation, chemotherapy and/or radiation and chemotherapy. Exemplary non-myeloablative methods can include for example, mini-transplant or reduced intensity conditioning.

In one aspect, the myeloablative radiation is received as a total body irradiation.

In one aspect, the radiation is received as a fractionated dose in two or more fractions. In another embodiment, the radiation is received as a fractionated dose in a hyperfractionation therapy. In another aspect, the radiation is received as a fractionated dose in an accelerated fractionation therapy.

In one aspect, the effective dose of IL-12 is given in one or more doses of 50 to 300 ng/Kg. Other effective doses of IL-12 are in the dose range of 100-200 ng/kg.

In one aspect, the one or more effective dose(s) of IL-12 are given before HSCT. In other aspects, the one or more effective dose(s) of IL-12 are given before and after HSCT. In another aspect, the one or more effective dose(s) of IL-12 are given after HSCT.

In one aspect, the one or more effective doses of IL-12 are administered topically, subcutaneously, intradermally, intravenously, intraperitoneally, intramuscularly, epidurally, parenterally, intranasally, and/or intracranially.

In one aspect, as an exemplary method and/or composition, the instant disclosure provided a comparison between the hematopoiesis-promoting activities of rMuIL-12 and a bone marrow cell transplant (BMCT) in irradiated mice in vivo, and demonstrated the potential cellular targets of rHuIL-12 and the role of IL-12 receptors in human hematopoiesis in vitro.

In another aspect, as an exemplary method and/or composition, the instant disclosure demonstrated that at least one administration of low-dose (10 ng/mouse) rMuIL-12 to lethally irradiated mice increased survival and peripheral blood cell recovery as effectively as a BMCT. In one embodiment, at 12 days post radiation, murine bone marrow of mice treated with rMuIL-12 was characterized with the presence of IL-12 receptor β2 subunit (IL-12Rβ2)-expressing myeloid progenitors, megakaryocytes, and osteoblasts.

In one aspect, as an exemplary method and/or composition, administration of rMuIL-12 also increased the number of IL-12Rβ2 expressing cells in mouse bone marrow Lin− cells. In one embodiment, analysis of human bone marrow cells indicated that pluripotent Lin− cells and CD34+ cells also expressed IL-12Rβ2 along with other markers of hematopoietic stem cells (HSCs).

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this Brief Summary, which is included for purposes of illustration only and not restriction. Additional embodiments may be disclosed in the Detailed Description below.

### BRIEF DESCRIPTION OF THE FIGURES

FIGS. 1A-1D describes the efficacy of rMuIL-12 in increasing the Survival (A) and Blood Cell Recovery (B-D) in irradiated mice to a Similar Extent as Bone Marrow Cell Transplant (BMCT). Animals received vehicle, rMuIL-12 (10 ng/mouse), or BMCT (1.1×10⁶ cells) intravenously and were monitored for survival and blood cell counts for 35 days. TBI=total body irradiation.

FIGS. 2A-2D. rMuIL-12 Increased Hematopoietic Reconstitution In Irradiated Mice. Sections of femoral bone marrow stained for IL-12Rβ2 are shown for non-irradiated, untreated mice (A) and, at 12 days post-TBI, for animals treated subcutaneously with vehicle or rMuIL-12 (20 ng/mouse). While mice treated with vehicle lacked IL-12Rβ2-expressing cells and showed no signs of hematopoietic regeneration (B), mice treated with rMuIL-12 showed hematopoietic reconstitution and the presence of IL-12Rβ2-expressing megakaryocytes, myeloid progenitors, and osteoblasts (C-D). Note hematopoiesis and IL-12Rβ2-expressing stem and non-stem cells after rMuIL-12 treatment. Magnification=100×.

FIG. 3. Megakaryocyte Islands Observed Close to the Trabecular bone. Sections of femoral bone marrow stained for IL-12Rβ2 (orange color) at 12 days post-TBI, for mice treated subcutaneously with rMuIL-12 (20 ng/mouse) at 24 hours before and 3 days after. Magnification=100×.

FIG. 4. Mouse Bone Marrow Lin− Cells Expressed IL-12Rβ2. Lin− cells were immunomagnetically selected among mouse bone marrow cells and were analyzed with flow cytometry following labeling with an antibody against IL-12Rβ2. Gated area (R6) indicates the subset expressing IL-12Rβ2, totaling approximately 5% of Lin− cells.

FIGS. 5A-5C. Human Bone Marrow Lin− Cells and CD34+ Cells Expressed IL-12Rβ2. Human Lin− cells (A) and CD34+ cells (B, C) were labeled with antibodies against IL-12Rβ2 and CD34 and analyzed with flow cytometry (A and B) or immunocytochemistry (C). The quadrants were set using unstained and isotype controls. R2: IL-12Rβ2+CD34−, R3: IL12Rβ2+CD34+, R5: IL-12Rβ2−CD34+.

FIGS. 6A-6B. Human Lin−IL-12Rβ2+ Cells and CD34+IL-12Rβ2 Cells Co Expressed Other Stem Cell Markers. Lin− cells (A) and CD34+ cells (B) were co-labeled with antibodies against IL-12Rβ2 and the indicated markers of stem cells and were analyzed with flow cytometry. Note that IL-12Rβ2 is expressed on 1-4% of Lin− cells and 6-50% of CD34+ cells.

FIG. 7. Survival of rhesus monkeys following exposure to TBI and treatment 24 hours after TBI with either vehicle or rHuIL-12.

Kaplan-Meier plots of survival over the study period are shown for each treatment group. Each dose group comprised 18 animals. Log rank p-values were 0.0305 0.0344, 0.0404, and 0.0265, respectively for the 50 ng/kg, 100 ng/kg, 250 ng/kg and 500 ng/kg dose groups vs. the vehicle-treated control group.

FIGS. 8A-8E. Blood counts over time in rhesus monkeys exposed to lethal TBI and treated 24 hours after TBI with either vehicle or rHuIL-12 (Average±SEM).

A) platelets; B) mean platelet volume; C) neutrophils; D) lymphocytes; E) reticulocytes. Normal ranges are as follows: lymphocytes, 1.85 to 8.71×109/L; neutrophils, 1.21 to 10.29×109/L; platelets, 252 to 612×109/L; mean platelet volume, 6.3 to 9.4×109/L; reticulocytes, 29.9 to 103.9×109/L.

FIGS. 9A-9D. Identification of Bone Marrow Regeneration Islands.

(A) Histopathological identification of regenerating bone marrow. Clusters of cells appearing in otherwise ablated bone marrow were scored as one regenerating island. Left panel, ablated bone marrow; middle panel, regenerating bone marrow; right panel, non-irradiated bone marrow. (Olympus BX41 compound microscope; Infinity Analyze software v5.0, magnification: 10×). (B) Quantification of number of islands of regeneration for individual treatment groups (left panel, p<0.01 for 500 ng/kg group vs. control) and the combined rHUIL-12-treated groups vs. vehicle-treated control (right panel, p<0.05). (C) Quantification of area of regeneration for individual treatment groups (left panel, p<0.05 for 50 and 500 ng/kg groups vs. control) and the combined rHUIL-12-treated groups vs. vehicle-treated control (right panel, p<0.05). (D) Quantification of megakaryocytes for individual treatment groups (left panel) and the combined rHUIL-12-treated groups vs. vehicle-treated control (right panel).

FIG. 10. rMuIL-12 increased percentage survival as effectively as a BMT in lethally irradiated mice. Mice were treated intravenously with vehicle, rMuIL-12×1 (10 ng administered 24 hours before TBI), rMuIL-12×2 (10 ng administered 24 hours before and 3 days after TBI), or BMT (1.1×106 cells administered 2 hours after TBI). Survival curves for each group were statistically compared by Mantel-Cox test.

FIGS. 11A-11C. Blood cell recovery in Lethally Irradiated Mice Treated with rMuIL-12 and BMT were comparable. Mice were treated with rMuIL-12×1 (10 ng administered 24 hours before TBI), rMuIL-12×2 (10 ng administered 24 hours before and 3 days after TBI), or BMT (1.1×106 cells) administered 2 hours after TBI. Blood counts for A) Neutrophils; B) RBCs; C) platelets were determined on Days 21, 28, and 35. Statistical analyses were performed using Student T-test. The dashed lines in panel A, B and C indicate normal levels in mice. Error bars represent mean±standard deviation.

FIGS. 12A-12B. Human Bone Marrow CD34+ Cells Express IL-12Rbeta2. A) Human CD34+ cells were labeled with antibodies against IL-12Rbeta2 and analyzed using immunocytochemistry. Analyses were performed on an Olympus BX41 compound microscope, ×200 (20× objective and 10× ocular lens) B) IL-12Rbeta2 expression on human bone marrow CD34+ cells analyzed by flow cytometry (Dot plots shown). Isotype matched control for each antibody was included. One representative image of three independent analyses is shown.

FIGS. 13A-13D. IL-12Rbeta2 co-expresses with Other Stem Cell Markers on CD34+ Cells from Normal Human Bone Marrow. CD34+ cells were co-labeled with antibodies against IL-12R 2 and the indicated stem cell markers and analyzed by flow cytometry. A) CD117 (ckit); B) CD135 (Flt3); C) CD133; D) CD318 (CDCP1). For each set of results, the left panel shows the isotype control. One representative image of three independent analyses is shown.

### DETAILED DESCRIPTION

Methods and compositions for hematopoietic recovery following transplantation, including hematopoietic stem cell transplantation (HSCT) are provided. Hematopoietic stem cell transplantation (HSCT) includes the transplantation of multipotent hematopoietic stem cells, derived from bone marrow, peripheral blood stem cells, or umbilical cord blood. As used herein the HSCT myeloablative methods can include the use of radiation, chemotherapy and/or radiation and chemotherapy. The methods and compositions can also be useful for the non-myeloablative methods, such as mini-transplant or reduced intensity conditioning.

Aspects and embodiments of the instant disclosure provide therapeutic compositions and methods of use thereof comprising IL-12, including recombinant human interleukin-12 (IL-12) preparation for hematopoietic recovery following transplantation, including hematopoietic stem cell transplantation (HSCT).

As an exemplary composition and/or method, a recombinant human interleukin-12 (rHuIL-12) and its mouse homologue, IL-12 (rMuIL-12), show a remarkable ability to reconstitute bone marrow using a single, low dose administered either before or after total body irradiation (TBI). These novel, surprising and unexpected findings provided evidence that rHuIL-12 directly acts on IL-12 receptors expressed on HSC and niche cells to stimulate hematopoiesis. Additional clinical studies confirm the efficacy of the use of rHuIL-12 as an adjunct to hematopoietic cellular transplants for enhancing HSC engraftment and bone marrow recovery following transplantation.

The present disclosure also relates to methods and therapeutic agents that can improve hematopoietic recovery following HSCT. As an example, one or more effective dose(s) of IL-12 can be administered before HSCT. In other examples, the one or more effective dose(s) of IL-12 are given before and after HSCT. In another example, the one or more effective dose(s) of IL-12 are given after HSCT.

In addition, the present disclosure relates to methods of protecting a subject from system, organ, tissue, or cellular damage, following exposure of the subject to ionizing radiation comprising: administering a dose of therapeutically effective amount of a pharmaceutical composition comprising substantially isolated IL-12 to the subject following myoablation and non-myoablation. Myeloablative methods can include, for example, radiation, chemotherapy and/or radiation and chemotherapy. Exemplary non-myeloablative methods can include for example, mini-transplant or reduced intensity conditioning.

This disclosure also relates to myeloablative radiation, which may be received, for example, as a total body irradiation, or through irradiation of a part of the body. The radiation may also be received as a fractionated dose in two or more fractions. In another embodiment, the radiation is received as a fractionated dose in a hyperfractionation therapy. In another aspect, the radiation is received as a fractionated dose in an accelerated fractionation therapy.

In one aspect, the effective dose of IL-12 is given in one or more doses of 100 to 300 ng/Kg.

In one aspect, the one or more effective doses of IL-12 are administered topically, subcutaneously, intradermally, intravenously, intraperitoneally, intramuscularly, epidurally, parenterally, intranasally, and/or intracranially.

This invention also relates to methods for comparing the hematopoiesis-promoting activities of recombinant IL-12 and a bone marrow cell transplant (BMCT) in irradiated subjects in vivo, and demonstrating the potential cellular targets of rHuIL-12 and the role of IL-12 receptors in human hematopoiesis in vitro.

This invention also relates to at least one administration of low-dose (10 ng/mouse) rMuIL-12 to lethally irradiated mice increased survival and peripheral blood cell recovery as effectively as a BMCT. In one embodiment, at 12 days post radiation, murine bone marrow of mice treated with rMuIL-12 was characterized with the presence of IL-12 receptor β2 subunit (IL-12Rβ2)-expressing myeloid progenitors, megakaryocytes, and osteoblasts.

This invention also relates to administration of rMuIL-12 to increase the number of IL-12Rβ2 expressing cells in mouse bone marrow Lin− cells. In one embodiment, analysis of human bone marrow cells indicated that pluripotent Lin− cells and CD34+ cells also expressed IL-12Rβ2 along with other markers of hematopoietic stem cells (HSCs).

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this Brief Summary, which is included for purposes of illustration only and not restriction. Additional embodiments may be disclosed in the Detailed Description below.

As used herein, IL-12 is a heterodimeric cytokine, comprising both p40 and p35 subunits, that is well-known for its role in immunity. In numerous reports spanning about two decades, IL-12 has been shown to have an essential role in the interaction between the innate and adaptive arms of immunity by regulating inflammatory responses, innate resistance to infection, and adaptive immunity. Endogenous IL-12 is required for resistance to many pathogens and to transplantable and chemically induced tumors. The hallmark effect of IL-12 in immunity is its ability to stimulate the production of interferon-gamma (IFN-gamma) from natural killer (NK) cells, macrophages and T cells. Further, several in vitro studies in the early-mid nineties reported that IL-12 is capable of stimulating hematopoiesis synergistically with other cytokines. The hematopoiesis-promoting activity of IL-12 appears to be due to a direct action on bone marrow stem cells as these studies used highly purified progenitors or even single cells. The role of IFN-gamma in the hematopoietic activity of IL-12 is not clear as several studies have linked both the promotion and suppression of hematopoiesis to IFN-gamma.

Interleukin-12 (IL-12) is shown to have a radioprotective function when used before or shortly after exposure to total body radiation (Neta, et al. (1994) IL-12 protects bone marrow from and sensitizes intestinal tract to ionizing radiation. J Immunol 153: 4230-4237; Chen, et al, (2007) IL-12 facilitates both the recovery of endogenous hematopoiesis and the engraftment of stem cells after ionizing radiation, Exp Hematol 35: 203-213; in addition, the entire disclosures of US20110206635 and U.S. Pat. No. 7,939,058 are herein incorporated by reference. In the studies, mice were rescued from the deleterious effects of lethal total body radiation. The radioprotective effect was reported to reside within an unknown cell population in the bone marrow, likely long-term repopulating hematopoietic stem cells. In another study, IL-12 was shown to provide early recovery peripheral blood cell counts following sublethal radiation of tumor-bearing mice (Basile, et al (2008) Multilineage hematopoietic recovery with concomitant antitumor effects using low dose Interleukin-12 in myelosuppressed tumor-bearing mice. J Transl Med 6: 26). In this latter study, it was shown that IL-12 was synergistic with radiation in reducing tumor volume. In particular, IL-12 did not to increase tumor volumes when administered either before or after radiation exposure.

Thus, IL-12 has potential in radioprotection of the bone marrow following total body radiation. However, early studies reported that although IL-12 had a radioprotective effect in the bone marrow, the gastrointestinal (GI) system was sensitized to radiation damage (Neta, et al.). In a later report, the GI sensitization effect of IL-12 was found to be dependent on the dose of IL-12 administered (Chen, et al.). There have been no reports of the radioprotective effects of IL-12 to other tissues or organs, other than bone marrow.

The present invention is based a surprising and unexpected discovery that certain murine recombinant IL-12 (e.g. m-HemaMax) and human recombinant IL-12 (e.g. HemaMax) have the ability to improve hematopoietic recovery following HSCT transplantation in a subject.

Hematopoietic stem cell transplant (HSCT) is a procedure that restores stem cells that have been destroyed by high doses of chemotherapy and/or radiation therapy. Patients who undergo total body irradiation (TBI) for stem cell transplantation have prolonged periods of low counts of platelets. These low platelet counts cause bleeding and infection. Thus far, no drug is available for use to speed the recovery of platelets, and therefore transfusions are often necessary.

Disease, disorders and/or conditions that can be treated by HSCT can include, for example, multiple Myeloma; Non-Hodgkin lymphoma (NHL); Hodgkin lymphoma; acute myeloid leukemia; Neuroblastoma; Germ cell tumors; Auto immune disorders; Amyloidosis

Autologous HSCT: Acute myeloid leukemia; Acute lymphoblastic leukemia; Chronic myeloid leukemia; Chronic lymphocytic leukemia; Myeloproliferative disorders; Myelodysplastic syndromes; Multiple myeloma; Non-Hodgkin lymphoma; Hodgkin disease; Aplastic anemia;

Allogeneic HSCT; Pure red cell aplasia; Paroxysmal nocturnal hemoglobinuria; Fanconi anemia; Thalassemia major; Sickle cell anemia; Severe combined immunodeficiency (SCID); Wiskott-Aldrich syndrome; Hemophagocytic lymphohistiocytosis (HLH); Inborn errors of metabolism

In certain embodiments, a BMT procedure specifically developed for patients who had previously not been considered suitable for a conventional BMT is a reduced intensity conditioning (“RIC”). The concept of the RIC transplant is that high-dose therapy may not be necessary in order to have the patient accept a donor's stem cells. This avoidance of high-dose therapy makes the procedure safer in patients of older age or with pre-existing health problems. Instead, patients receive relatively less toxic conditioning therapy. Depending on the degree of reduction, the conditioning therapy is sometimes given in the Outpatient Unit rather than admitting the patient to the Inpatient Unit. The reduced-intensity conditioning is designed to suppress the patient's immune system enough so that it will accept the donor stem cells.

In one aspect, bone marrow is completely destroyed by total body irradiation or a combination of high dose chemotherapy and total body irradiation. The purpose of such extreme treatments is to eliminate all diseased cells that may reside in the bone marrow (e.g. leukemia cells or metastasized tumor cells derived from solid tumors). The procedure is followed by transplantation of bone marrow stem/progenitor cells.

In one aspect, adult stem/progenitor cells used for re-populating the empty bone cavity may be obtained directly from the bone marrow (for example, from posterior iliac crests), or from peripheral blood. In the latter case, the donor (e.g. the patient himself/herself or a close relative) may be pretreated with G-CSF and/or GM-CSF to mobilize bone marrow cells and enhance the yield of peripheral blood progenitor cells. The stem/progenitor cell population may be enriched by various methods, for example by using magnetic-activated cell sorting to remove monocytes or T-lymphocytes or Ficoll-Hypaque density gradient centrifugation. Prior to transplantation, the stem/progenitor cells are usually stored in a 5-20% dimethylsulfoxide-containing medium such as Iscove's modified Dulbecco's medium in the vapor phase of liquid nitrogen. Any standardized procedures for the isolation, enrichment and storage of stem/progenitor cells that are well known in the art may be used.

Leading hematopoietic supportive care therapies (EPO) have received black box warnings in response to their effect on tumor growth. The direct mechanism of action of HemaMax on hematopoietic stem cells can be contrasted with other well-known hematopoietic growth factors, such as EPO (branded as Procrit, Aranesp, and Epogen), and G-CSF (branded as Neulasta and Neupogen), as well as TPO mimetics (branded as Nplate and Promacta) and IL-11 (branded as Neumega). EPO-like molecules act at the level of erythroid precursor cells yielding increases in red blood cells. G-CSF-like molecules act at the level of neutrophil precursor cells yielding increases in neutrophils. TPO mimetics and IL-11 act at the level of megakaryoctes leading to increases in platelets. Target cell populations of these hematopoietic growth factors are all downstream of the hematopoietic stem cell, which is HemaMax's target cell.

There is no overlap between HemaMax's mechanism of action and that of the well-known hematopoeitic growth factors. HemaMax's mechanism of action involves activation of hematopoietic stem cells upstream of the activity of other hematopoietic factors, Consequently, HemaMax can replenish and regenerate the hematopoietic and immune systems following ablation, whereas these downstream acting factors cannot, as they target precursor cells to yield a single blood cell type. Via this early-acting (upstream) mechanism, HemaMax's activation of primitive hematopoietic stem cells can restore all major blood cell types. In pre-clinical studies, HemaMax has anti-tumor effects given its immunotherapy mechanism of action (increase in INF-γ and upregulation of T and NK cells).

The murine counterpart to HemaMax (rMuIL-12) promotes full-lineage blood cell recovery including white and red blood cells and platelets in both normal and tumor-bearing mice exposed to sublethal or lethal Total Body Irradiation (TBI). The activity of HemaMax is initiated at the level of primitive cells (hematopoietic and non-hematopoietic stem cells) residing in the bone marrow compartment. Activation of these primitive cells leads to regeneration of the bone marrow compartment following myeloablation or myelosuppression caused by radiation or chemotherapy.

HemaMax has a unique role in re-defining current methods pre/post transplantation of stem cells prior to HSCT and as an adjuvant Hematopoietic Stem Cell (HSC) engraftment enhancer post-HSCT. HSCT is most commonly used in the treatment of leukemia and lymphoma (also neuroblastoma and multiple myeloma) and most effective when in remission. HemaMax could restoring stem cells/bone marrow destroyed by treatments of chemotherapy by stimulating renewal and differentiation of early hematopoietic stem cells (HSCs—mobilize prior to transplantation and aid in HSC engraftment post-transplantation).

For the purpose of the current disclosure, the following definitions shall in their entireties be used to define technical terms and to define the scope of the composition of matter for which protection is sought in the claims.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, apes, and prenatal, pediatric, and adult humans.

As used herein, “preventing” or “protecting” means preventing in whole or in part, or ameliorating or controlling.

As used herein, the term “treating” refers to both therapeutic treatment and prophylactic or preventative measures, or administering an agent suspected of having therapeutic potential.

The term “a pharmaceutically effective amount” as used herein means an amount of active compound or pharmaceutical agent that elicits the biological or medicinal response in a tissue, system, animal or human that is being sought by a researcher, veterinarian, medical doctor or other clinician, which includes alleviation or palliation of the symptoms of the disease being treated.

As used herein, an “effective amount” in reference to the pharmaceutical compositions of the instant disclosure refers to the amount sufficient to have utility and provide desired therapeutic endpoint.

As used herein, radiation induced damage following total body irradiation (TBI) can affect organ, tissues, systems associated with the following: bone marrow, lymphatic system, immune system, mucosal tissue, mucosal immune system, gastrointestinal system, cardiovascular system, nervous system, reproductive organs, prostate, ovaries, lung, kidney, skin and brain.

As used herein, radiation exposure may be associated with radiation-induced acute, chronic, and systemic damage effects. In one aspect, the instant disclosure provides therapeutic compositions and methods of use thereof for treating radiation induced acute damage effects. Exemplary damage effects are not always limited to the normal tissue in the irradiation beam. Exemplary damage effect can extend beyond the treated area and can include, for example, esophagitis (difficulty swallowing); pneumonitis (cough, fever, lung fluid accumulation) in the lung; intestinal irradiation-induced inflammation (diarrhea, cramps, abdominal pain); nausea and vomiting; tiredness, fatigue, diarrhea, headache, tissue swelling, skin erythema, cough, and difficulty breathing. Exemplary damage effects can affect areas of the skin e.g. erythema, desquamation; oral mucosa, e.g. mucositis, nasopharynx; oropharynx; vocal cord; tonsil; skin, (squamous or carcinoma). In certain embodiments, exemplary effects can include telangiectasia, fibrosis, spinal cord myelitis, and cartilage fibrosis.

In certain embodiments, exemplary radiation induced damage effects can also include Blood-forming organ (Bone marrow) syndrome, characterized by damage to cells that divide at the most rapid pace (such as bone marrow, the spleen and lymphatic tissue). Exemplary symptoms include internal bleeding, fatigue, bacterial infections, and fever.

In certain embodiments, exemplary radiation induced damage effects can also include gastrointestinal tract syndrome, characterized by damage to cells that divide less rapidly (such as the linings of the stomach and intestines). Exemplary symptoms include nausea, vomiting, diarrhea, dehydration, electrolytic imbalance, loss of digestion ability, bleeding ulcers, and the symptoms of blood-forming organ syndrome.

In certain embodiments, exemplary radiation-induced damage effects can also include mucositis. In one embodiment, the radiation-induced mucositis is oral mucositis.

In certain embodiments, exemplary radiation induced effects can also include central nervous system syndrome, characterized by damage to cells that do not reproduce such as nerve cells. Exemplary symptoms include loss of coordination, confusion, coma, convulsions, shock, and the symptoms of the blood forming organ and gastrointestinal tract syndromes.

In certain embodiments, exemplary radiation induced damage effects can also include effects on the fetus due to prenatal radiation exposure. An embryo/fetus is especially sensitive to radiation, (embryo/fetus cells are rapidly dividing), particularly in the first 20 weeks of pregnancy.

In certain embodiments, exemplary radiation induced effects can also include damages due to ionizing irradiation-induced production of radical oxygen species (ROS) including superoxide, hydroxyl radical, nitric oxide and peroxynitrite from the interaction of ionizing irradiation with oxygen and water.

In one aspect, the instant disclosure provides therapeutic compositions and methods of use thereof for treating radiation induced chronic damage effects. Chronic irradiation effects are critically important in all patients, but particularly in those who receive total body irradiation (TBI). Total body irradiation is utilized in some cancer therapies particularly for patients who require a bone marrow transplant.

Exemplary radiation induced chronic damage effects can include, for example, features common to premature aging such as hair graying, skin thinning and dryness, formation of cataracts, early myocardial fibrosis, myocardial infarction, neurodegeneration, osteopenia/osteomalasia and neurocognitive defects.

In certain embodiments, exemplary radiation induced effects can also include fibrosis (the replacement of normal tissue with scar tissue, leading to restricted movement of the affected area); damage to the bowels, causing diarrhea and bleeding; memory loss; infertility and/or carcinogenesis/leukemogenesis.

In certain embodiments, the methods and compositions of the present disclosure are useful for improving hematopoiesis following stem cell transplantation. Exemplary myeloablative delivery modality/regimen can include, for example, conventional fractionation therapy, hyperfractionation, hypofractionation, and accelerated fractionation.

In one embodiment, the therapeutic modality/regimen is hyperfractionation therapy. In hyperfractionation, the goal is to deliver higher tumor doses while maintaining a level of long-term tissue damage that is clinically acceptable. The daily dose is unchanged or slightly increased while the dose per fraction is decreased, and the overall treatment time remains constant.

In one embodiment, the therapeutic modality/regimen is accelerated fractionation therapy. In the accelerated fractionation therapy, the dose per fraction is unchanged while the daily dose is increased, and the total time for the treatment is reduced.

In one embodiment, the therapeutic modality/regimen is Continuous hyperfractionated accelerated radiation therapy (CHART) therapy. In (CHART) therapy, an intense schedule of treatment in which multiple daily fractions are administered within an abbreviated period.

In one embodiment, the therapeutic modality/regimen is IMRT.

Combination with Chemotherapy

A number of chemotherapeutic agents can enhance the effects of radiation therapy. In one aspect, the aspects and embodiments of the present disclosure can be utilized as a combined therapy with existing chemotherapeutic modalities. The combination (sequential or concurrent) therapy can be co-administration or co-formulation.

“Interleukin-12 (IL-12)” refers to IL-12 molecule that yields at least one of the hematopoietic properties disclosed herein, including native IL-12 molecules, variant 11-12 molecules and covalently modified IL-12 molecules, now known or to be developed in the future, produced in any manner known in the art now or to be developed in the future.

The IL-12 molecule may be present in a substantially isolated form. It will be understood that the product may be mixed with carriers or diluents which will not interfere with the intended purpose of the product and still be regarded as substantially isolated. A product of the invention may also be in a substantially purified form, in which case it will generally comprise about 80%, 85%, or 90%, including, for example, at least about 95%, at least about 98% or at least about 99% of the peptide or dry mass of the preparation.

Generally, the amino acid sequences of the IL-12 molecule used in embodiments of the invention are derived from the specific mammal to be treated by the methods of the invention. Thus, for the sake of illustration, for humans, generally human IL-12, or recombinant human IL-12, would be administered to a human in the methods of the invention, and similarly, for felines, for example, the feline IL-12, or recombinant feline IL-12, would be administered to a feline in the methods of the invention.

Also included in the invention, however, are certain embodiments where the IL-12 molecule does not derive its amino acid sequence from the mammal that is the subject of the therapeutic methods of the invention. For the sake of illustration, human IL-12 or recombinant human IL-12 may be utilized in a feline mammal. Still other embodiments of the invention include IL-12 molecules where the native amino acid sequence of IL-12 is altered from the native sequence, but the IL-12 molecule functions to yield the hematopoietic properties of IL-12 that are disclosed herein. Alterations from the native, species-specific amino acid sequence of IL-12 include changes in the primary sequence of IL-12 and encompass deletions and additions to the primary amino acid sequence to yield variant IL-12 molecules. An example of a highly derivatized IL-12 molecule is the redesigned IL-12 molecule produced by Maxygen, Inc. (Leong S R, et al., Proc Nati Acad Sci USA. 2003 Feb. 4; 100 (3): 1163-8.), where the variant IL-12 molecule is produced by a DNA shuffling method. Also included are modified IL-12 molecules are also included in the methods of invention, such as covalent modifications to the IL-12 molecule that increase its shelf life, half-life, potency, solubility, delivery, etc., additions of polyethylene glycol groups, polypropylene glycol, etc., in the manner set forth in U.S. Pat. Nos. 4,640,835; 4,496,689; 4,301,144; 4,670,417; 4,791,192 or 4,179,337. One type of covalent modification of the IL-12 molecule is introduced into the molecule by reacting targeted amino acid residues of the IL-12 polypeptide with an organic derivatizing agent that is capable of reacting with selected side chains or the N- or C-terminal residues of the IL-12 polypeptide. Both native sequence IL-12 and amino acid sequence variants of IL-12 may be covalently modified. Also as referred to herein, the IL-12 molecule can be produced by various methods known in the art, including recombinant methods. Other IL-12 variants included in the present disclosure are those where the canonical sequence is post-translationally-modified, for example, glycosylated. In certain embodiments, the IL-12 is expressed in a mammalian expression system or cell line. In one embodiment, the IL-12 is produced by expression in Chinese Hamster Ovary (CHO) cells.

Since it is often difficult to predict in advance the characteristics of a variant IL-12 polypeptide, it will be appreciated that some screening of the recovered variant will be needed to select the optimal variant. A preferred method of assessing a change in the hematological stimulating or enhancing properties of variant IL-12 molecules is via the lethal irradiation rescue protocol disclosed below. Other potential modifications of protein or polypeptide properties such as redox or thermal stability, hydrophobicity, susceptibility to proteolytic degradation, or the tendency to aggregate with carriers or into multimers are assayed by methods well known in the art.

For general descriptions relating IL-12, see U.S. Pat. Nos. 5,573,764, 5,648,072, 5,648,467, 5,744,132, 5,756,085, 5,853,714 and 6,683,046. Interleukin-12 (IL-12) is a heterodimeric cytokine generally described as a proinflammatory cytokine that regulates the activity of cells involved in the immune response (Fitz K M, et al., 1989, J. Exp. Med. 170:827-45). Generally IL-12 stimulates the production of interferon-γ (INF-γ) from natural killer (NK) cells and T cells (Lertmemongkolchai G, Cai, et al., 2001, Journal of Immunology. 166:1097-105; Cui J, Shin T, et al., 1997, Science. 278:1623-6; Ohteki T, Fukao T, et alk., 1999, J. Exp. Med. 189:1981-6; Airoldi I, Gri G, et al., 2000, Journal of Immunology. 165:6880-8), favors the differentiation of T helper 1 (TH1) cells (Hsieh C S, et al., 1993, Science. 260:547-9; Manetti R, et al., 1993, J. Exp. Med. 177:1199-1204), and forms a link between innate resistance and adaptive immunity. IL-12 has also been shown to inhibit cancer growth via its immuno-modulatory and anti-angiogenesis effects (Brunda M J, et al., 1993, J. Exp. Med. 178:1223-1230; Noguchi Y, et al., 1996, Proc. Natl. Acad. Sci. U.S.A. 93:11798-11801; Giordano P N, et al., 2001, J. Exp. Med. 194:1195-1206; Colombo M P, et al, 2002, Cytokine Growth factor rev. 13:155-168; Yao L, et al., 2000, Blood 96:1900-1905). IL-12 is produced mainly by dendritic cells (DC) and phagocytes (macrophages and neutrophils) once they are activated by encountering pathogenic bacteria, fungi or intracellular parasites (Reis C, et al., 1997, J. Exp. Med. 186:1819-1829; Gazzinelli R T, et al., 1994, J. Immunol. 153:2533-2543; Dalod M, et al., 2002, J. Exp. Med. 195:517-528). The IL-12 receptor (IL-12 R) is expressed mainly by activated T cells and NK cells (Presky D H, et al., 1996, Proc. Natl. Acad. Sci. U.S.A. 93:14002-14007; Wu C Y, et al., 1996, Eur J. Immunol. 26:345-50).

Generally the production of IL-12 stimulates the production of INF-γ, which, in turn, enhances the production of IL-12, thus forming a positive feedback loop. In in vitro systems, it has been reported that IL-12 can synergize with other cytokines (IL-3 and SCF for example) to stimulate the proliferation and differentiation of early hematopoietic progenitors (Jacobsen S E, et al., 1993, J. Exp Med 2: 413-8; Ploemacher R E, et al., 1993, Leukemia 7: 1381-8; Hirao A, et al., 1995, Stem Cells 13: 47-53).

In vivo administration of IL-12 was observed to decrease peripheral blood cell counts and bone marrow hematopoiesis (Robertson M J, et al., 1999, Clinical Cancer Research 5: 9-16; Lenzi R, et al., 2002, Clinical Cancer Research 8:3686-95; Ryffel B. 1997, Clin Immunol Immunopathol. 83:18-20; Car B D, et al., 1999, The Toxicol Pathol. 27:58-63). Using INF-γ receptor knockout mice, Eng, et al and Car, et al demonstrated that high dose IL-12 did not induce the commonly seen toxicity effect, i.e., there was no inhibition of hematopoiesis (Eng V M, et al., 1995, J. Exp Med. 181:1893-8; Car B D, et al., 1995, American Journal of Pathology 147:1693-707). This observation suggests that the general phenomenon of IL-12 facilitated enhancement of differentiated hematopoietic cells, as reported previously, may be balanced in vivo by the production of INF-γ, which acts in a dominant myelo-suppressive fashion.

Current evidence suggests that an exemplary IL-12 preparation, a recombinant human IL-12 (e.g., HemaMax), triggers responses at, at least, 4 levels in the body (see FIG. 14). At the Level 1 response, HemaMax promotes proliferation and activation of extant, radiosensitive immune cells, namely NK cells, macrophages, and dendritic cells. HemaMax-induced plasma elevations of IL-15 and IL-18 also facilitate maturation of NK cells, leading to the release of IFN-γ, which in turn, positively affects the production of endogenous IL-12 from macrophages and dendritic cells, and perhaps NK cells. These events enhance the innate immune competency early on following HemaMax administration. At the Level 2 response, HemaMax promotes proliferation and differentiation of the surviving hematopoietic stem cells, osteoblasts, and megakaryocytes into a specific cellular configuration that ensues optimal hematopoiesis. HemaMax-induced secretion of EPO from CD34+, IL-12Rβ2-positive bone marrow cells may also suppress local over-production of IFN-γ in the bone marrow and, thus, provide a milieu that promotes expansion of hematopoietic cells. Hematopoietic regeneration in the bone marrow enhances both innate and adaptive immune competency. At the Level 3 response, HemaMax preserves GI stem cells, leading to a reduction in pathogen leakage, an increase in food consumption, and a decrease in diarrhea. At the Level 4 response, HemaMax likely directly increases renal release of EPO, a cytoprotective factor, which enhances cellular viability in a diverse set of organs/tissues. Continued production of endogenous IL-12 primarily from dendritic cells activated by pathogens and/or EPO serves as a positive feedback loop and plays a key role in sustaining the initial response to exogenous HemaMax, perhaps for weeks after radiation.

### Methods of Administration of IL-12

The instant disclosure provides methods of treatment by administration to a subject of one or more effective dose(s) of IL-12 for a duration to achieve the desired therapeutic effect. The subject is preferably a mammal, including, but not limited to, animals such as cows, pigs, horses, chickens, cats, dogs, etc., and is most preferably human.

Various delivery systems are known and can be used to administer IL-12 in accordance with the methods of the invention, e.g., encapsulation in liposomes, microparticles, microcapsules, recombinant cells capable of expressing IL-12, receptor-mediated endocytosis (see, e.g., Wu and Wu, 1987, J. Biol. Chem. 262:4429-4432), construction of nucleic acid comprising a gene for IL-12 as part of a retroviral or other vector, etc. Methods of introduction include but are not limited to intradermal, intramuscular, intraperitoneal, intravenous, subcutaneous, intranasal, epidural, and oral routes.

IL-12 can be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce pharmaceutical compositions comprising IL-12 into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent. It may he desirable to administer the pharmaceutical compositions comprising IL-12 locally to the area in need of treatment; this may be achieved, for example and not by way of limitation, by topical application, by injection, by means of a catheter, by means of a suppository, or by means of an implant, said implant being of a porous, non-porous, or gelatinous material, including membranes, such as sialastic membranes, or fibers.

Other modes of IL-12 administration involve delivery in a vesicle, in particular a liposome (see Langer, Science 249:1527-1533 (1990); Treat, et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

Still other modes of administration of IL-12 involve delivery in a controlled release system. In certain embodiments, a pump may be used (see Langer, supra; Sefton, CRC Crit. Ref. Biomed. Eng. 14:201 (1987); Buchwald, et al., Surgery 88:507 (1980); Saudek, et al., N. Engl. J. Med. 321:574 (1989)). Additionally polymeric materials can be used (see Medical Applications of Controlled Release, Langer and Wise (eds.), CRC Pres, Boca Raton, Fla. (1974); Controlled Drug Bioavailability, Drug Product Design and Performance, Smolen and Ball (eds.), Wiley, N.Y. (1984); Ranger and Peppas, J. Macromol. Sci. Rev. Macromol. Chem. 23:61 (1983; see also Levy, et al., Science 228:190 (1985); During, et al., Ann. Neurol. 25:351 (1989); Howard, et al., J. Neurosurg. 71:105 (1989)), or a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)). Other controlled release systems are discussed in the review by Langer (Science 249:1527-1533 (1990)).

### Forms and Dosages of IL-12

Suitable dosage forms of IL-12 for use in embodiments of the present invention encompass physiologically acceptable carriers that are inherently non-toxic and non-therapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and PEG, Carriers for topical or gel-based forms of IL-12 polypeptides include polysaccharides such as sodium carboxymethylcellulose or methylcellulose, polyvinylpyrrolidone, polyacrylates, polyoxyethylene-polyoxypropylene-block polymers, PEG, and wood wax alcohols, For all administrations, conventional depot forms are suitably used. Such forms include, for example, microcapsules, nano-capsules, liposomes, plasters, inhalation forms, nose sprays, sublingual tablets, and sustained-release preparations.

Suitable examples of sustained-release preparations include semipermeable matrices of solid hydrophobic polymers containing the polypeptide, which matrices are in the form of shaped articles, e.g. films, or microcapsules, Examples of sustained-release matrices include polyesters, hydrogels (for example, poly(2-hydroxyethyl-methacrylate) as described by Langer, et al., supra and Langer, supra, or poly(vinylalcohol), polylactides (U.S. Pat. No. 3,773,919), copolymers of L-glutamic acid and .gamma. ethyl-L-glutamate (Sidman, et al, supra), non-degradable ethylene-vinyl acetate (Langer, et al., supra), degradable lactic acid-glycolic acid copolymers such as the Lupron Depot™ (injectable microspheres composed of lactic acid-glycolicacid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated IL-12 polypeptides remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Sustained-release IL-12 containing compositions also include liposomally entrapped polypeptides. Liposomes containing a IL-12 polypeptide are prepared by methods known in the art, such as described in Eppstein, et al., Proc. Natl. Acad. Sci. USA 82:3688-3692 (1985); Hwang, et al., Proc. Natl. Acad. Sci. USA 77:4030 (1980); and U.S. Pat. Nos. 4,485,045 and 4,544,545. Ordinarily, the liposomes are the small (about 200-800 Angstroms) unilamelar type in which the lipid content is greater than about 30 mol. % cholesterol, the selected proportion being adjusted for the optimal Wnt polypeptide therapy. Liposomes with enhanced circulation time are disclosed in U.S. Pat. No. 5,013,556.

For the treatment of disease, the appropriate dosage of a IL-12 polypeptide will depend on the type of disease to be treated, as defined above, the severity and course of the disease, previous therapy, the patient's clinical history and response to the IL-12 therapeutic methods disclosed herein, and the discretion of the attending physician. In accordance with the invention, IL-12 is suitably administered to the patient at one time or over a series of treatments.

Depending on the type and severity of the disease, about 10 ng/kg to 2000 ng/kg of IL-12 is an initial candidate dosage for administration to the patient, whether, for example, by one or more separate administrations, or by continuous infusion. Humans can safely tolerate a repeated dosages of about 500 ng/kg, but single dosages of up to about 200 ng/kg should not produce toxic side effects. For example, the dose may be the same as that for other cytokines such as G-CSF, GM-CSF and EPO. For repeated administrations over several days or longer, depending on the condition, the treatment is sustained until a desired suppression of disease symptoms occurs. However, other dosage regimens may be useful. The progress of this therapy is easily monitored by conventional techniques and assays.

IL-12 may be administered along with other cytokines, either by direct co-administration or sequential administration. When one or more cytokines are co-administered with IL-12, lesser doses of IL-12 may be employed. Suitable doses of other cytokines, i.e. other than IL-12, are from about 1 ug/kg to about 15 mg/kg of cytokine. For example, the dose may be the same as that for other cytokines such as G-CSF, GM-CSF and EPO. The other cytokine(s) may be administered prior to, simultaneously with, or following administration of IL-12. The cytokine(s) and IL-12 may be combined to form a pharmaceutically composition for simultaneous administration to the mammal. In certain embodiments, the amounts of IL-12 and cytokine are such that a synergistic repopulation of blood cells (or synergistic increase in proliferation and/or differentiation of hematopoietic cells) occurs in the mammal upon administration of IL-12 and other cytokine thereto. In other words, the coordinated action of the two or more agents (i.e. the IL-12 and one or more cytokine(s)) with respect to repopulation of blood cells (or proliferation/differentiation of hematopoietic cells) is greater than the sum of the individual effects of these molecules.

Therapeutic formulations of IL-12 are prepared for storage by mixing IL-12 having the desired degree of purity with optional physiologically acceptable carriers, excipients, or stabilizers (Remington's Pharmaceutical Sciences, 16th edition, Osol, A., Ed., (1980)), in the form of lyophilized cake or aqueous solutions. Acceptable carriers, excipients, or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counter-ions such as sodium; and/or non-ionic surfactants such as Tween®, Pluronics™ or polyethylene glycol (PEG).

The term “buffer” as used herein denotes a pharmaceutically acceptable excipient, which stabilizes the pH of a pharmaceutical preparation. Suitable buffers are well known in the art and can be found in the literature. Pharmaceutically acceptable buffers include but are not limited to histidine-buffers, citrate-buffers, succinate-buffers, acetate-buffers, phosphate-buffers, arginine-buffers or mixtures thereof. The abovementioned buffers are generally used in an amount of about 1 mM to about 100 mM, of about 5 mM to about 50 mM and of about 10-20 mM. The pH of the buffered solution can be at least 4.0, at least 4.5, at least 5.0, at least 5.5 or at least 6.0. The pH of the buffered solution can be less than 7.5, less than 7.0, or less than 6.5. The pH of the buffered solution can be about 4.0 to about 7.5, about 5.5 to about 7.5, about 5.0 to about 6.5, and about 5.5 to about 6.5 with an acid or a base known in the art, e.g. hydrochloric acid, acetic acid, phosphoric acid, sulfuric acid and citric acid, sodium hydroxide and potassium hydroxide. As used herein when describing pH, “about” means plus or minus 0.2 pH units.

As used herein, the term “surfactant” can include a pharmaceutically acceptable excipient which is used to protect protein formulations against mechanical stresses like agitation and shearing. Examples of pharmaceutically acceptable surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulphate (SDS). Suitable surfactants include polyoxyethylenesorbitan-fatty acid esters such as polysorbate 20, (sold under the trademark Tween 20®) and polysorbate 80 (sold under the trademark Tween 80®). Suitable polyethylene-polypropylene copolymers are those sold under the names Pluronic® F68 or Poloxamer 188®. Suitable Polyoxyethylene alkyl ethers are those sold under the trademark Brij®. Suitable alkylphenolpolyoxyethylene esthers are sold under the trade name Triton-X. When polysorbate 20 (Tween 20®) and polysorbate 80 (Tween 80®) are used they are generally used in a concentration range of about 0.001 to about 1%, of about 0.005 to about 0.2% and of about 0.01% to about 0.1% w/v (weight/volume).

As used herein, the term “stabilizer” can include a pharmaceutical acceptable excipient, which protects the active pharmaceutical ingredient and/or the formulation from chemical and/or physical degradation during manufacturing, storage and application. Chemical and physical degradation pathways of protein pharmaceuticals are reviewed by Cleland, et al., Crit. Rev. Ther. Drug Carrier Syst., 70(4):307-77 (1993); Wang, Int. J. Pharm., 7S5(2): 129-88 (1999); Wang, Int. J. Pharm., 203(1-2): 1-60 (2000); and Chi, et al, Pharm. Res., 20(9): 1325-36 (2003). Stabilizers include but are not limited to sugars, amino acids, polyols, cyclodextrines, e.g. hydroxypropyl-beta-cyclodextrine, sulfobutylethyl-beta-cyclodextrin, beta-cyclodextrin, polyethylenglycols, e.g. PEG 3000, PEG 3350, PEG 4000, PEG 6000, albumine, human serum albumin (HSA), bovine serum albumin (BSA), salts, e.g. sodium chloride, magnesium chloride, calcium chloride, chelators, e.g. EDTA as hereafter defined. As mentioned hereinabove, stabilizers can be present in the formulation in an amount of about 10 to about 500 mM, an amount of about 10 to about 300 mM, or in an amount of about 100 mM to about 300 mM. In some embodiments, exemplary IL-12 can he dissolved in an appropriate pharmaceutical formulation wherein it is stable.

IL-12 also may be entrapped in microcapsules prepared, for example, by coacervation techniques or by interfacial polymerization (for example, hydroxymethylcellulose or gelatin-microcapsules and poly-(methylmethacylate)microcapsules, respectively), in colloidal drug delivery systems (for example, liposomes, albumin microspheres, microemulsions, nano-particles, and nanocapsules), or in macroemulsions. Such techniques are disclosed in Remington's Pharmaceutical Sciences, supra.

IL-12 to be used for in vivo administration must be sterile. This is readily accomplished by filtration through sterile filtration membranes, prior to or following lyophilization and reconstitution. IL-12 ordinarily will be stored in lyophilized form or in solution. Therapeutic IL-12 compositions generally are placed into a container having a sterile access port, for example, an intravenous solution bag or vial having a stopper pierceable by a hypodermic injection needle.

When applied topically, IL-12 is suitably combined with other ingredients, such as carriers and/or adjuvants. There are no limitations on the nature of such other ingredients, except that they must be physiologically acceptable and efficacious for their intended administration, and cannot degrade the activity of the active ingredients of the composition. Examples of suitable vehicles include ointments, creams, gels, or suspensions, with or without purified collagen. The compositions also may be impregnated into transdermal patches, plasters, and bandages, preferably in liquid or semi-liquid form.

For obtaining a gel formulation, IL-12 formulated in a liquid composition may be mixed with an effective amount of a water-soluble polysaccharide or synthetic polymer such as PEG to form a gel of the proper viscosity to be applied topically. The polysaccharide that may be used includes, for example, cellulose derivatives such as etherified cellulose derivatives, including alkyl celluloses, hydroxyalkyl celluloses, and alkylhydroxyalkyl celluloses, for example, methylcellulose, hydroxyethyl cellulose, carboxymethyl cellulose, hydroxypropyl methylcellulose, and hydroxypropyl cellulose; starch and fractionated starch; agar; alginic acid and alginates; gum arable; pullullan; agarose; carrageenan; dextrans; dextrins; fructans; inulin; mannans; xylans; arabinans; chitosans; glycogens; glucans; and synthetic biopolymers; as well as gums such as xanthan gum; guar gum; locust bean gum; gum arabic; tragacanth gum; and karaya gum; and derivatives and mixtures thereof. The preferred gelling agent herein is one that is inert to biological systems, nontoxic, simple to prepare, and not too runny or viscous, and will not destabilize the IL-12 molecule held within it.

Preferably the polysaccharide is an etherified cellulose derivative, more preferably one that is well defined, purified, and listed in USP, e.g., methylcellulose and the hydroxyalkyl cellulose derivatives, such as hydroxypropyl cellulose, hydroxyethyl cellulose, and hydroxypropyl methylcellulose. Most preferred herein is methylcellulose.

The polyethylene glycol useful for gelling is typically a mixture of low and high molecular weight PEGs to obtain the proper viscosity. For example, a mixture of a PEG of molecular weight 400-600 with one of molecular weight 1500 would be effective for this purpose when mixed in the proper ratio to obtain a paste.

The term “water soluble” as applied to the polysaccharides and PEGs is meant to include colloidal solutions and dispersions. In general, the solubility of the cellulose derivatives is determined by the degree of substitution of ether groups, and the stabilizing derivatives useful herein should have a sufficient quantity of such ether groups per anhydroglucose unit in the cellulose chain to render the derivatives water soluble. A degree of ether substitution of at least 0.35 ether groups per anhydroglucose unit is generally sufficient. Additionally, the cellulose derivatives may be in the form of alkali metal salts, for example, the Li, Na, K, or Cs salts.

If methylcellulose is employed in the gel, preferably it comprises about 2-5%, more preferably about 3%, of the gel and IL-12 is present in an amount of about 300-1000 mg per ml of gel.

An effective amount of IL-12 to be employed therapeutically will depend, for example, upon the therapeutic objectives, the route of administration, and the condition of the patient. Accordingly, it will be necessary for the therapist to titer the dosage and modify the route of administration as required to obtain the optimal therapeutic effect. Typically, the clinician will administer IL-12 until a dosage is reached that achieves the desired effect. A typical dosage for systemic treatment might range from about 10 ng/kg to up to 2000 ng/kg or more, depending on the factors mentioned above. In some embodiments, the dose ranges can be from about 1, 2, 3, 4, 5, 6, 7, 8, 9, 10, 11, 12, 13, 14, 15, 16, 17, 18, 19 to about 20; to about 30; to about 50; to about 100, to about 200, to about 300 or to about 500 ng/kg. In one aspect, the dose is less than 500 ng/kg, In another aspect, the dose is less than 300 ng/kg. In another aspect, the dose is less than about 200 ng/kg. In another aspect, the dose is less than about 100 ng/kg. In another aspect, the dose is less than about 50 ng/kg. In other aspects, the dose can range from about 10 to 300 ng/kg, 20 to 40 ng/kg, 25 to 35 ng/kg, 50 to 100 ng/kg.

In one aspect, exemplary therapeutic compositions described herein can be administered in combination with fractionation therapy. In one embodiment, the therapeutically effective dose is given before each fraction. In one embodiment, the therapeutically effective dose is given at about the same time as the administration of each fraction. In one embodiment, the therapeutically effective dose is given before each fraction, ranging from 5, 10, 15, 20, 25, 30, 35, 40 50, or 60 minutes before each fraction; or 2, 3, 4, 5, 6, 7, 8, 9, 10, 11, 12 hours after each fraction; or 1, 2, 3, 4, 5, 6, 7 days before each fraction. In one embodiment, the therapeutically effective dose is given after each fraction, ranging from 5, 10, 15, 20, 25, 30, 35, 40 50, or 60 minutes after each fraction; or 2, 3, 4, 5, 6, 7, 8, 9, 10, 11, 12 hours after each fraction; or 1, 2, 3, 4, 5, 6, 7 days after each fraction; or once, twice, three times, 4 times, 5 times, 6 times, 7 times weekly, biweekly, or bimonthly, during or after the radiation treatment. In another embodiment, one or more exemplary doses of IL-12 is administered (1 to 100 ng/kg) at about 5, 10, 15, 20, 30, 40, 50, 60 min, 1, 2, 3, 4, 5, 6, 7, 8, 9, 10, 11, 12, 13, 14, 15, 16, 17, 18, 19, 20, 21, 22, 23, 24 hours, 1 day, 2 days, 3 days, 4 days, 5 days, 6 days, 7 days both before and after each radiation dose in fractionated regimens of 1 to 10 doses/day for up to 30 days, administered either as TBI or locally, using each respective radiation source.

As an alternative general proposition, the IL-12 receptor is formulated and delivered to the target site or tissue at a dosage capable of establishing in the tissue an IL-12 level greater than about 0.1 ng/cc up to a maximum dose that is efficacious but not unduly toxic. This intra-tissue concentration should be maintained if possible by the administration regime, including by continuous infusion, sustained release, topical application, or injection at empirically determined frequencies. The progress of this therapy is easily monitored by conventional assays.

“Near the time of administration of the treatment” refers to the administration of IL-12 at any reasonable time period either before and/or after the administration of the treatment, such as about one month, about three weeks, about two weeks, about one week, several days, about 120 hours, about 96 hours, about 72 hours, about 48 hours, about 24 hours, about 20 hours, several hours, about one hour or minutes. Near the time of administration of the treatment may also refer to either the simultaneous or near simultaneous administration of the treatment and IL-12, i.e., within minutes to one day.

“Chemotherapy” refers to any therapy that includes natural or synthetic agents now known or to be developed in the medical arts. Examples of chemotherapy include the numerous cancer drugs that are currently available. However, chemotherapy also includes any drug, natural or synthetic, that is intended to treat a disease state. In certain embodiments of the invention, chemotherapy may include the administration of several state of the art drugs intended to treat the disease state. Examples include combined chemotherapy with docetaxel, cisplatin, and 5-fluorouracil for patients with locally advanced squamous cell carcinoma of the head (Tsukuda, M., et al., Int J Clin Oncol. 2004 June; 9 (3): 161-6), and fludarabine and bendamustine in refractory and relapsed indolent lymphoma (Konigsmann M, et al, Leuk Lymphoma. 2004; 45 (9): 1821-1827).

As used herein, exemplary sources of therapeutic or accidental ionizing radiation can include, for example, alpha, beta, gamma, x-ray, and neutron sources.

“Radiation therapy” refers to any therapy where any form of radiation is used to treat the disease state. The instruments that produce the radiation for the radiation therapy are either those instruments currently available or to be available in the future.

“High dose treatment modalities” refer to treatments that are high sub-lethal or near lethal. High dose treatment modalities are intended to have an increased ability to achieve therapeutic endpoint, but generally possess increased associated toxicities. Further, generally high dose treatment modalities exhibit increased hematopoietic damage, as compared with conventional treatment modalities. The protocols for high dose treatment modalities are those currently used or to be used in the future.

As used herein, radiation therapy “treatment modality” can include both ionizing and non-ionizing radiation sources. Exemplary ionizing radiation treatment modality can include, for example, external beam radiotherapy; Intensity modulated radiation therapy (IMRT); Image Guided Radiotherapy (IGRT); X Irradiation (e.g. photon beam therapy); electron beam (e.g. beta irradiation); proton irradiation; high linear energy transfer (LET) particles; stereotactic radiosurgery; gamma knife; linear accelerator mediated frameless stereotactic radiosurgery; robot arm controlled x irradiation delivery system; radioisotope radiotherapy for organ specific or cancer cell specific uptake; radioisotope bound to monoclonal antibody for tumor targeted radiotherapy (or radioimmunotherapy, RIT); brachytherapy (interstitial or intracavity) high dose rate radiation source implantation; permanent radioactive seed implantation for organ specific dose delivery.

“A dose dense treatment regimen” is generally a treatment regimen whereby the treatment is repeated sequentially in an accelerated manner to achieve the desired treatment outcome, as compared with conventional treatment regimens. The methods of the invention facilitate the use of dose dense treatment regimens by reducing or ameliorating the associated hematopoietic toxicities of the treatment, thereby permitting dose dense treatment regimens to be utilized and increasing the rate of success in treating a particular disease state. (see generally, Hudis C A, Schmits N, Semin Oncol. 2004 June; 31 (3 Suppl 8): 19-26; Keith B, et al., J Clin Oncol. 2004 Feb. 15; 22 (4): 749; author reply 751-3; Maurel J, et al, Cancer. 2004 Apr. 1; 100 (7): 1498-506; Atkins C D, J Clin Oncol. 2004 Feb. 15; 22 (4): 749-50.)

“Chemoprotection or radioprotection” refers to protection from, or an apparent decrease in, the associated hematopoietic toxicity of a treatment intended to target the disease state.

As used herein, “Acute Radiation Syndrome (ARS) (also known as radiation toxicity or radiation sickness), is characterized by an acute illness caused by receiving lethal or sublethal irradiation of the entire body (or most of the body) by a high dose of penetrating radiation in a very short period of time (e.g. a matter of minutes). Examples of people who suffered from ARS are the survivors of the Hiroshima and Nagasaki atomic bombs, the firefighters that first responded after the Chernobyl Nuclear Power Plant event in 1986, and some unintentional exposures to sterilization irradiators. In certain embodiments, the radiation dose associated with acute radiation syndrome is usually large (i.e., greater than 0.7 Gray (Gy) or 70 rads). In certain embodiments, mild symptoms may be observed with doses as low as 0.3 Gy or 30 rads.

As used herein, “acute damage effects” and “damage effects” can include radiation induced damage due to acute lethal and near lethal radiation dose.

“Solid tumors” generally refers to the presence of cancer of body tissues other than blood, bone marrow, or the lymphatic system.

“Hematopoietic disorders (cancers)” generally refers to the presence of cancerous cells originated from hematopoietic system.

“Ameliorate the deficiency” refers to a reduction in the hematopoietic deficiency, i.e., an improvement in the deficiency, or a restoration, partially or complete, of the normal state as defined by currently medical practice. Thus, amelioration of the hematopoietic deficiency refers to an increase in, a stimulation, an enhancement or promotion of, hematopoiesis generally or specifically. Amelioration of the hematopoietic deficiency can be observed to be general, i.e., to increase two or more hematopoietic cell types or lineages, or specific, i.e., to increase one hematopoietic cell type or lineages.

“Bone marrow cells” generally refers to cells that reside in and/or home to the bone marrow compartment of a mammal. Included in the term “bone marrow cells” is not only cells of hematopoietic origin, including but not limited to hematopoietic repopulating cells, hematopoietic stem cell and/or progenitor cells, but any cells that may be derived from bone marrow, such as endothelial cells, mesenchymal cells, bone cells, neural cells, supporting cells (stromal cells), including but not limited to the associated stem and/or progenitor cells for these and other cell types and lineages.

“Hematopoietic cell type” generally refers to differentiated hematopoietic cells of various types, but can also include the hematopoietic progenitor cells from which the particular hematopoietic cell types originate from, such as various blast cells referring to all the cell types related to blood cell production, including stem cells, progenitor cells, and various lineage cells, such as myeloid cells, lymphoid cell, etc.,

“Hematopoietic cell lineage” generally refers to a particular lineage of differentiated hematopoietic cells, such as myeloid or lymphoid, but could also refer to more differentiated lineages such as dendritic, erythroid, etc.

“IL-12 facilitated proliferation” of cells refers to an increase, a stimulation, or an enhancement of hematopoiesis that at least partially attributed to an expansion, or increase, in cells that generally reside or home to the bone marrow of a mammal, such as hematopoietic progenitor and/or stem cells, but includes other cells that comprise the microenvironment of the bone marrow niche.

“Stimulation or enhancement of hematopoiesis” generally refers to an increase in one or more hematopoietic cell types or lineages, and especially relates to a stimulation or enhancement of one or more hematopoietic cell types or lineages in cases where a mammal has a deficiency in one or more hematopoietic cell types or lineages.

“Hematopoietic long-term repopulating cells” are generally the most primitive blood cells in the bone marrow; they are the blood stem cells that are responsible for providing life-long production of the various blood cell types and lineages.

“Hematopoietic stern cells” are generally the blood stem cells; there are two types: “long-term repopulating” as defined above, and “short-term repopulating” which can produce “progenitor cells” for a short period (weeks, months or even sometimes years depending on the mammal).

“Hematopoietic progenitor cells” are generally the first cells to differentiate from (i.e., mature from) blood stem cells; they then differentiate (mature) into the various blood cell types and lineages.

“Hematopoietic support cells” are the non-blood cells of the bone marrow; these cells provide “support” for blood cell production. These cells are also referred to as bone marrow stromal cells.

“Bone marrow preservation” means the process whereby bone marrow that has been damaged by radiation, chemotherapy, disease or toxins is maintained at its normal, or near normal, state; “bone marrow recovery” means the process whereby bone marrow that has been damaged by radiation, chemotherapy, disease or toxins is restored to its normal, near normal state, or where any measurable improvement in bone marrow function are obtained; bone marrow function is the process whereby appropriate levels of the various blood cell types or lineages are produced from the hematopoietic (blood) stem cells.

“Bone marrow failure” is the pathologic process where bone marrow that has been damaged by radiation, chemotherapy, disease or toxins is not able to be restored to normal and, therefore, fails to produce sufficient blood cells to maintain proper hematopoiesis in the mammal.

### EXAMPLES

The invention is now described with reference to the following Examples. These Examples are provided for the purpose of illustration only, and the invention is not limited to these Examples, but rather encompasses all variations that are evident as a result of the teaching provided herein.

Prior to the experiments described herein, there was no published protocol that allows for compositions and methods comprising IL-12, including recombinant human interleukin-12 (IL-12) preparation for improving hematopoietic recovery following HSCT.

Aspects and embodiments of the instant disclosure stem from the unexpected discovery that certain IL-12 formulations have surprising and unexpected utility and efficacy when administered to a subject following HSCT transplantation.

By way of example, a method to prepare therapeutically effective IL-12 formulation was developed.

### Example 1

Materials and Methods

rMuIL-12, Mouse BMCT, and Human Bone Marrow CD34+ Cells.

rMuIL-12 (recombinant mouse IL-12) was from Peprotech (Catalog #210-12), Rocky Hill, N.J., USA) or SBH Sciences (LS #45(Q4)) (Natick, Mass., USA) exclusively to Neumedicines. Studies in mice and mouse-derived bone marrow cells utilized rMuIL-12. BMCT was prepared by flushing out bone marrow cells from femurs and tibias of normal C57/BL6 mice with PBS using a 25G5/8 needle. Cells were filtered through a 40 μm cell strainer, washed with RPM1 containing 10% FBS and cryopreserved in liquid nitrogen. Human bone marrow cells expressing CD34 were purchased from Lonza Group, Ltd, (Walkersville, Md.).

Mice

Female (BMCT study) or male (bone marrow study) C57BL/6 mice were obtained from Jackson Laboratories (Sacramento, Calif.). Murine studies were conducted at BATTS Laboratories (Northridge, Calif.). Mice were maintained in quarantine for at least one week prior to the initiation of the studies. Mice used in the studies were 8 weeks to 10 weeks old and weighed approximately 20 g with no signs of disease. All procedures were reviewed and approved by BATTS Laboratories Institutional Animal Care and Use Committee (IACUC), which is accredited by the Association for the Assessment and Accreditation of Laboratory Animal Care (AAALAC) and the American Association of Laboratory Animal Care. During the study, care and use of animals were in accordance with principles outlined in the Guide for the Care and Use of Laboratory Animals published by the US National Institutes of Health (publication No: 85-23, revised 1996).

### Example 2

Survival Studies in Irradiated Mice Treated with rMuIL-12 and BMCT

At day 0, female C57BL/6 mice (n=40) were subjected to TBI at a lethal dose of 8.2 Gy using Gammacell® 40 with ¹³⁷Cs source (Theratronics, Ontario, Canada) in a specially constructed “pie-box” to keep mice in the center of the irradiator for even distribution of radiation. Mice received intravenous injections of vehicle (n=10) or rMuIL-12 at a dose of 10 ng/mouse at either 24 hours before TBI (n=10) or 24 hours before and 3 days after TBI (n=10). A fourth group of mice (n=10) received BMCT (1.1×10⁶ cells) 2 hours after irradiation. Mice were monitored for survival up to day 35. During this period, mice had access to sterilized food and acidified water ad libitum. Between-group differences in survival were evaluated with Kaplan-Meier survival analysis, followed by Mantel-Cox Test for survival time and Pearson's chi-square test for percent survival. Blood samples were withdrawn from half the animals in each group on day 28 and the other half on days 21 and 35 and analyzed by automated hematology analyzer (Hemavet; Drew Scientific Inc., Waterbury Conn.). Between-group differences in blood cell counts were analyzed by analysis of variance (ANOVA).

### Example 3

Mouse Bone Marrow Immunohistochemistry

Male C57BL/6 mice (n=2 per group) were subjected to TBI at 8.0 Gy and subsequently received vehicle or rMuIL-12 (20 ng/mouse) subcutaneously either one dose at 24 hours after irradiation or two doses at 24 hours and 2 days after irradiation. Mice were sacrificed 12 days after irradiation, and femoral bones were isolated, fixed in formalin and subsequently prepared as paraffin-embedded, sectioned tissues by Cyto-Pathology Diagnostic Center, Inc. (Duarte, Calif., USA).

Sectioned tissues were deparaffinized with xylene, rehydrated with decreasing concentrations of ethanol, and subjected to heat-induced epitope retrieval (HIER) to recover antigens. Endogenous peroxidase was inhibited with 0.3% H₂O₂ for 30 mins (VWR; San Francisco, Calif.), and background staining was blocked with Background Sniper (Biocare Medical, LLC; Concord, Calif.). Tissue sections were then incubated with rabbit anti-mouse IL-12R 2 (Sigma; St Louis, Mo.). Tissue sections were washed and incubated with peroxidase conjugated anti-rabbit IgG (ImmPRESS; Vector Laboratories; Burlingame, Calif.). Orange coloring of peroxidase labeled cells was developed following incubation with AEC substrate (ImmPACT AEC; Vector Laboratories; Burlingame, Calif.). Tissue was counterstained with CAT Hematoxylin (Biocare Medical, Concord, Calif.). Tissue sections were immersed in Vectamount (Vector Laboratories; Burlingame, Calif.), covered with a cover slip, sealed with clear nail polish, and visualized using an Olympus Compound microscope (Olympus America, Inc.; Center Valley, Pa.) at 20×-100× magnification.

### Example 4

Immunocytochemical Analysis of IL-12Rβ2 on Human CD34+ Cells

Human bone marrow CD34+ cells obtained from Lanza Group, Ltd, (Walkersville, Md.) were seeded on slides treated with 5 μg/mL fibronectin, were fixed in cold methanol for 10 minutes at −20° C., and, after treatment with Background Sniper (Biocare Medical; Concord, Calif.), were labeled with a rabbit polyclonal antibody against IL-12Rβ2 (Sigma; St Louis, Mo.) followed by incubation with anti-rabbit IgG coupled with horseradish peroxidase (ImmPRESS reagent; Vector Laboratories; Burlingame, Calif.). Slides were incubated with InimPACT AEC peroxidase substrate (Vector Laboratories; Burlingame, Calif.) for 30 minutes and were counterstained in CAT Hematoxylin (Biocare Medical, LLC; Concord, Calif.). The negative control included cells fixed and treated with the same reagents without the primary rabbit polyclonal antibody. Photographs were taken using an Olympus compound microscope.

### Example 5

Preparation of Mouse Hematopoietic Lin− and Lin−IL-12Rβ2+ Cells and Human Hematopoietic Lin− Cells

Mouse bone marrow was obtained from Bioreclamation (Liverpool, N.Y.) or from BATTS Laboratories. Cells were diluted with MACS buffer (Miltenyi Biotec; Auburn, Calif.) and filtered through a 70 μm cell strainer (VWR; San Francisco, Calif.). Cells were then washed in MACS buffer and incubated with biotin-conjugated monoclonal antibodies specific for lineage markers namely, CD3e, CD4, CD5, CD8b, CD8a, B220, CD11b, Grl and Ter (80) (Miltenyi Biotec; Auburn, Calif.). Cells were then washed and incubated with anti-biotin Microbeads. Lin+ cells were depleted by passing cells through a MACS column placed in the magnetic field of a QuadroMACS separator (Miltenyi Biotec; Auburn, Calif.). Magnetically labeled Lin+ cells are retained on the column while unlabeled Lin− cells were collected as effluent. In select studies, mouse bone marrow Lin− cells were sorted via flow cytometry on a MoFlow cell sorter (Beckman Coulter; Indianapolis, Ind.) to yield IL-12Rβ2+ cell population using the HAM10B9 antibody (BD Biosciences; San Jose, Calif.), which reacts with the unique β2 subunit (IL-12Rβ2) of the mouse IL-12 receptor complex.

Human bone marrow cells were obtained from Lonza Group, Ltd, (Walkersville, Md.). Cells were diluted with MACS buffer (Miltenyi Biotec; Auburn, Calif.) and filtered through a 70 μm cell strainer (VWR; San Francisco, Calif.). Bone marrow mononuclear cells were separated by density gradient centrifugation (455 g for 35 minutes at 4° C.) using Ficoll-Paque (GE Lifesciences; Piscataway, N.J.). Platelets were removed from the mononuclear fraction following an additional centrifugation at 200 g for 10 minutes. The resultant cell pellet was resuspended in MACS buffer and incubated with biotin-conjugated monoclonal antibodies specific for lineage markers CD2, CD3, CD 11b, CD14, CD15, CD I6, CD19, CD56, CD123 and CD235a (Glycophorin A) (Miltenyi Biotec; Auburn, Calif.). Lin− cells were collected following removal of Lin+ cells by magnetic beads as mentioned earlier.

### Example 6

How Cytometry Analysis of Mouse and Human Hematopoietic Cells

Mouse and human bone marrow Lin− cells were incubated with an allophycocyanin-conjugated anti-human/mouse IL-12Rβ2, clone #305719 (APC-IL-12Rβ2) antibody (R&D systems; Minneapolis, Minn.) for 30 minutes at room temperature. Unstained and isotype controls were included. In select studies, human bone marrow Lin− cells and CD34+ cells were also labeled with APC-IL-12Rβ2 antibody and conjugated monoclonal phycoerythrin (PE)-CD34 antibody, clone #563 (BD Biosciences; San Jose, Calif.), fluorescein isothiocyanate (FITC)-ckit, clone #104D2 (Abcam; Cambridge, Mass.), FITC-KDR, clone #89106 (R&D systems; Minneapolis, Minn.), PE-CD133, clone # EMK08 (eBioscience; San Diego, Calif.), PE-Flt-3, clone #4G8 (BD Biosciences; San Jose, Calif.), PE-CDI50, clone # A12 (BD Biosciences; San Jose, Calif.), PE-CDCP1 antibody, clone #309121 (R&D systems; Minneapolis, Minn.). Human bone marrow CD34+ cells were similarly incubated with APC-IL-12Rβ2 and PE-CD34 antibodies. Cells were then washed, resuspended in DPBS, and analyzed with a MoFlow flow cytometer (Beckman Coulter; Brea, Calif.). At least three different donors were evaluated for each analysis.

### Example 7

Results

rMuIL-12 Increases Survival and Promotes Blood Cell Recovery as Effectively as BMCT in Lethally Irradiated Mice

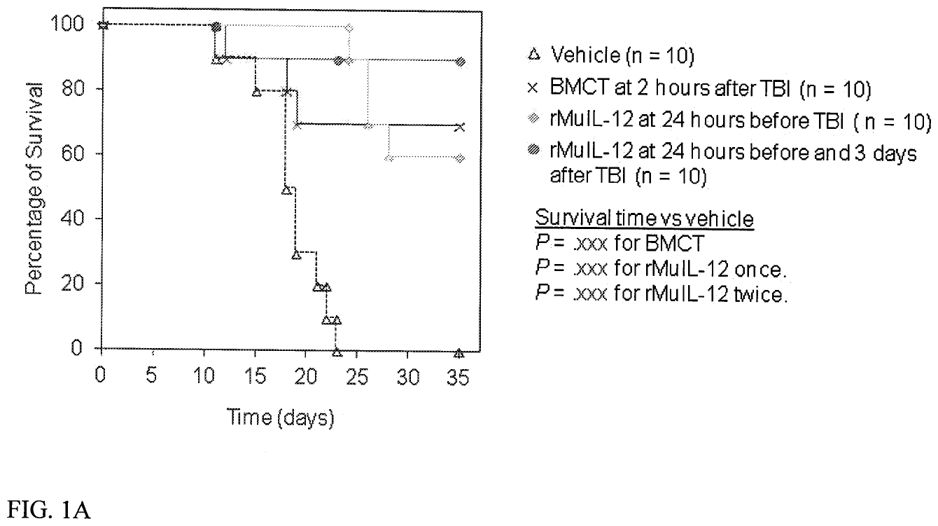

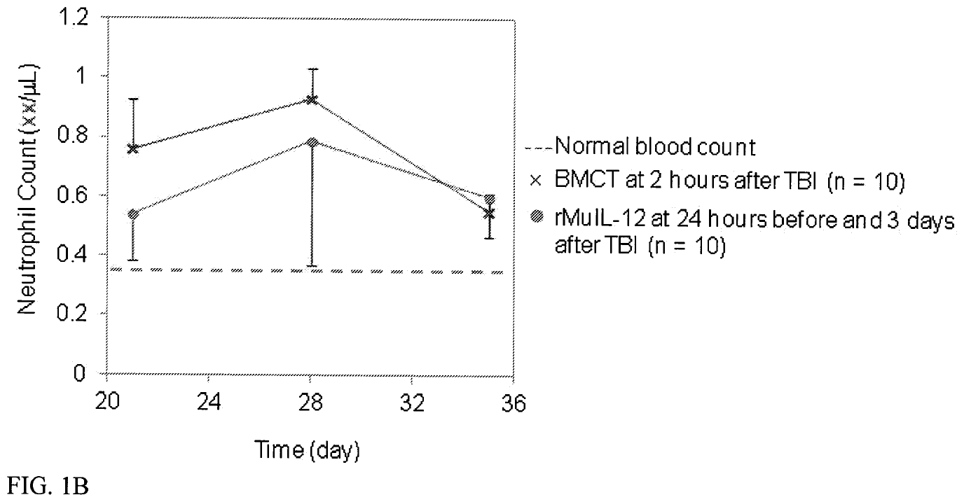

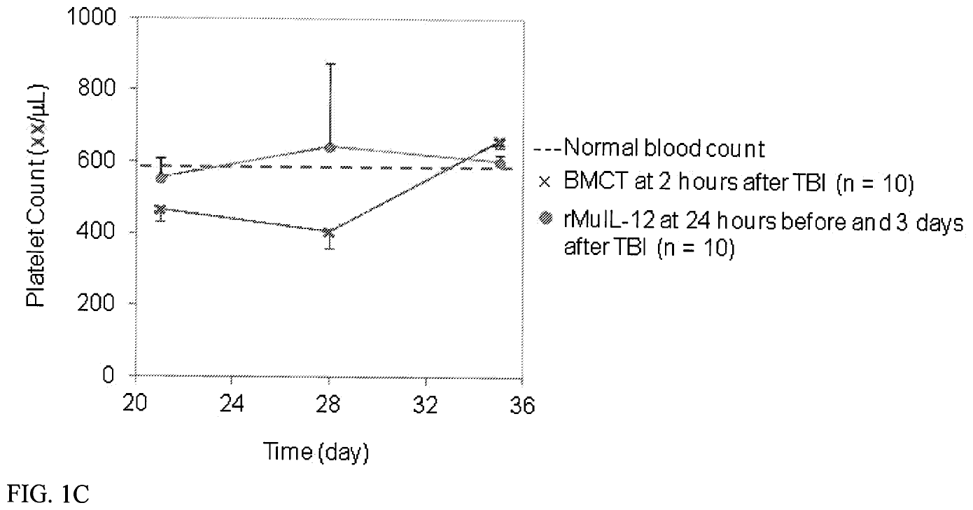

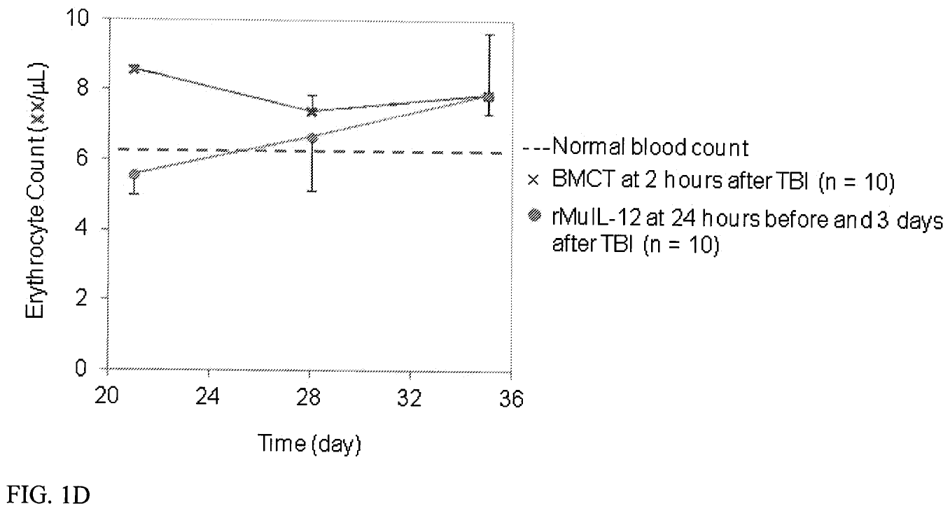

To begin to demonstrate the potential of rHuIL-12 for use in the clinical HSC transplantation setting, the survival and hematopoiesis-promoting effects of rMuIL-12 (10 ng/mouse) administered intravenously either once (at 24 hours before TBI) or twice (at 24 hours before and 3 days after TBI) was compared to those of BMCT (1.1×10⁶ whole bone marrow cells) administered 2 hours after TBI in lethally irradiated mice over a period of 35 days. BMCT and rMuIL-12 at both dosing schedules reduced death due to irradiation to essentially the same extent (FIG. 1a). Overall percentages of survival were 0% in the vehicle group, 70% in the BMCT group, 60% in the once rMuIL-12 group, and 90% in the twice rMuIL-12 group (FIG. 1a). Between-group differences in percent survival were significant when compared to the vehicle group (P<0.005). Between-group differences in survival were not statistically significant for the BMCT and rMuIL-12 groups

Analysis of blood cell counts in irradiated mice demonstrated that both BMCT and once or twice rMuIL-12 administrations increased peripheral blood cell counts to a similar extent (FIGS. 1b-d). Between-group differences in blood cell counts were not statistically significant for the BMCT and rMulL-12 groups. However, there were slight differences in recovery for the BMCT, rMuIL-12 dosed once, and rMuIL-12 dosed twice, groups. Notably, the platelet recovery for twice dosed rMuIL-12 groups reached normal platelet levels earlier than the BMCT group (FIG. 1d).

### Example 8

rMuIL-12 Promotes Hematopoiesis in Irradiated Mice Bone Marrow

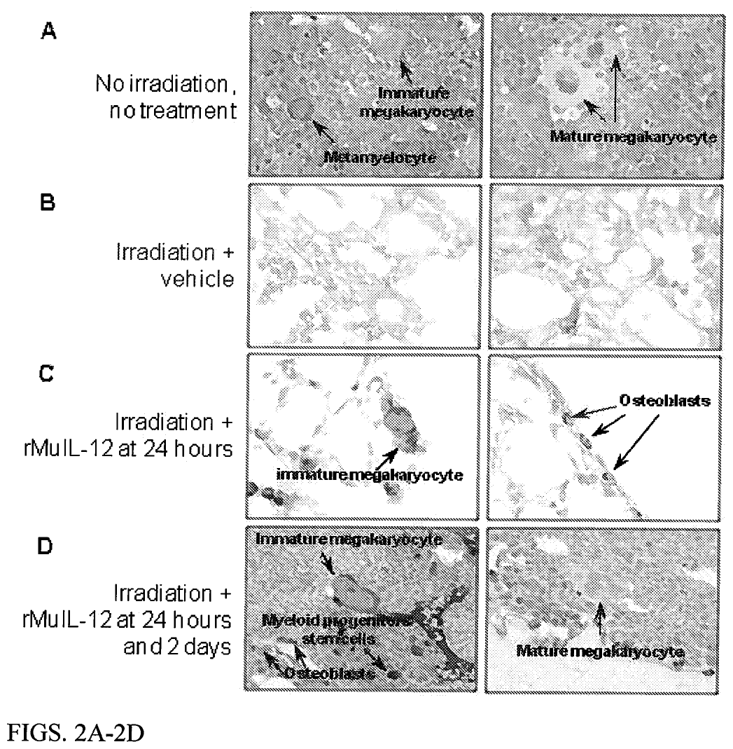

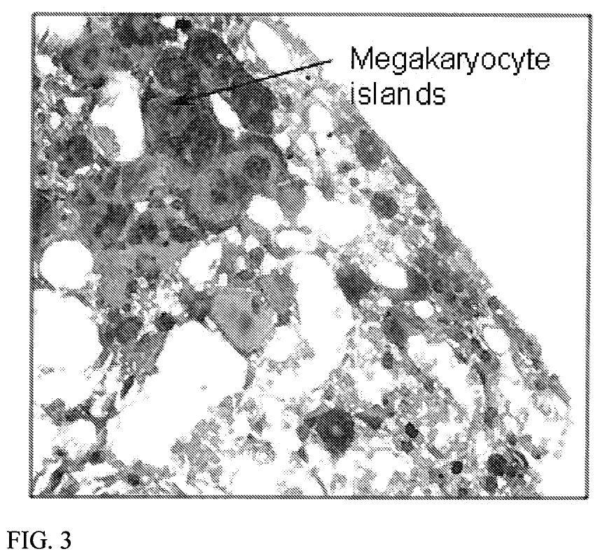

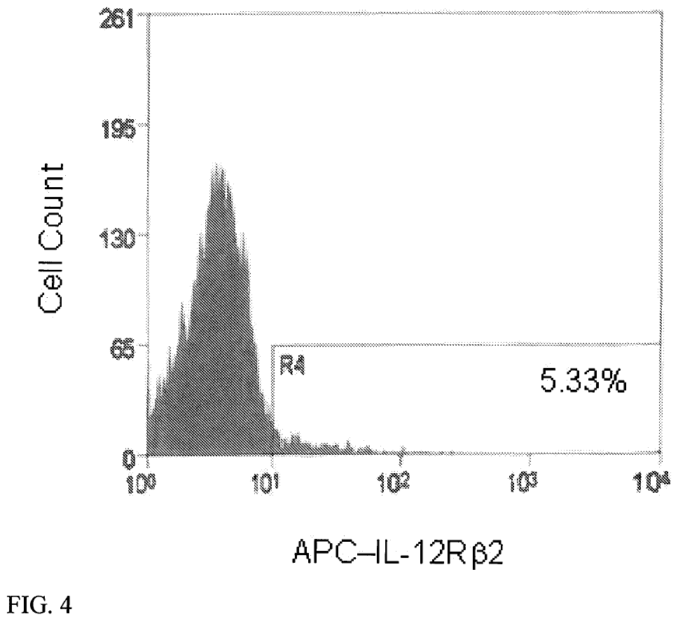

IL-12 functionality is driven by its interaction with its receptor, IL-12R, the beta 2 subunit (IL-12Rβ2), of which is a unique component of the receptor. We hypothesized that the regenerative ability of rMuIL-12 in lethally irradiated mice is driven by the expression of its receptor, mIL-12R on hematopoietic stem cells (HSCs). We further evaluated the hematopoiesis-promoting activity of rMuIL-12 by a phenotypic analysis of murine femoral bone marrow sections from lethally irradiated mice treated with rMuIL-12. Femoral bone marrow at 12 days and 30 days after lethal radiation was stained for IL-12Rβ2 from mice treated with rMuIL-12 (20 ng/mouse) or vehicle subcutaneously at 24 hrs after and 3 days after TBI. Bone marrow from irradiated, rMuIL-12-treated mice was characterized by the presence of IL-12Rβ2-expressing osteoblasts, progenitor cells, immature megakaryocytes with lobulated nuclei surrounded by a narrow rim of cytoplasm, matured megakaryocytes with lobulated nuclei and voluminous cytoplasm (FIG. 2a). In contrast, bone marrow from irradiated, vehicle-treated mice was characterized with minimal signs of hematopoietic regeneration and a complete lack of IL-12Rβ2-expressing cells at 12 days following irradiation (FIG. 2b). Myeloid progenitor and osteoblast cell types were analyzed by immunohistochemical analyses using Sca-1 and Osteocalcin markers respectively (data not shown). Osteoblasts have been shown to promote hematopoietic engraftment in mice in an allogeneic setting (EI-Badri N S, et al. “Osteoblasts Promote Engraftment Of Allogeneic Hematopoietic Stem Cells,” Exp Hematol 1998; 26:110-116.) and play a role in homing and expansion of megakaryocytes. (Ahmed N, et al., “Cytokine-Induced Expansion of Human CD34+ Stem/Progenitor and CD34+CD41+ Early Megakaryocytic Marrow Cells Cultured on Normal Osteoblasts,” Stem Cells 1999; 17:92-99; Dominici M, et al., “Restoration and Reversible Expansion of the Osteoblastic Hematopoietic Stem Cell Niche after Marrow Radioablation,” Blood 2009; I 14:2333-2343.) Consistent with this, we observed IL-12Rβ2 expressing megakaryocyte islands close to the endosteal surface in bone marrow of mice treated with rMuIL-12, dosed twice, at 24 hours after and 3 days after radiation (FIG. 3). This is in contrast to normal, untreated mice wherein the megakaryocytes occupy parasinusoidal sites. (Dominici M, et al., supra.) In order to confirm the expression of IL-12Rβ2 on the morphologically identified HSCs to support our results, Lin⁻cells were immuno-magnetically selected among mouse bone marrow cells. Lineage depleted cells (Lin−) represent a population of cells consisting of primitive stem cells and multipotent progenitors lacking mature lineage cell markers. Flow cytometry analysis demonstrated that approximately 0.5-7% of mouse Lin− cells expressed IL-12Rβ2 (FIG. 4).

### Example 9

In Vivo Treatment of Mice with rMuIL-12 Directly Increases the Number of IL-12Rβ2+ Cells Isolated from Bone Marrow Lin− Cells in the Absence of Radiation

The expression of mIL-12Rβ2 on mouse HSCs, megakaryocytes and osteoblasts, and the regenerative capacity of rMuIL-12 demonstrated by its ability to produce survival and hematopoietic recovery comparable to a BMCT in lethally irradiated mice suggest that the IL-12-mediated hematopoietic recovery and regeneration is triggered by its direct action on mIL-12Rβ2 expressing cells. Next we assessed the effects of rMuIL-12 on IL12Rβ2 expressing cells in mouse bone marrow in vivo. C57BL/6 mice were either treated with rMuIL-12 (10 ng/mouse) via tail vein injection or were not treated. Bone marrow was harvested 21 hours after treatment with rMuIL-12 for the first experiment, and at 25 hours post treatment with IL-12 for the second experiment. Lineage negative cells were isolated from each group and were further fractionated via flow cytometry sorting to yield Lin−IL-12Rβ2+. The relative percent of Lin−IL-12Rβ2+ cells present in the treated or untreated groups is shown in Table 1. Treatment with rMuIL-12 yields an increase in the relative number of Lin−IL-12Rβ2+ cells among lineage negative cells of the bone marrow compartment, and results in a 3.5 fold increase in IL-12Rβ2+ cells after 21 hrs and a 5.4 fold increase in IL-12Rβ2+ cells after 25 hrs.

### Example 10

Human Bone Marrow Lin− Cells and CD34+ Cells Express IL-12Rβ2 Along with Known HSC Markers

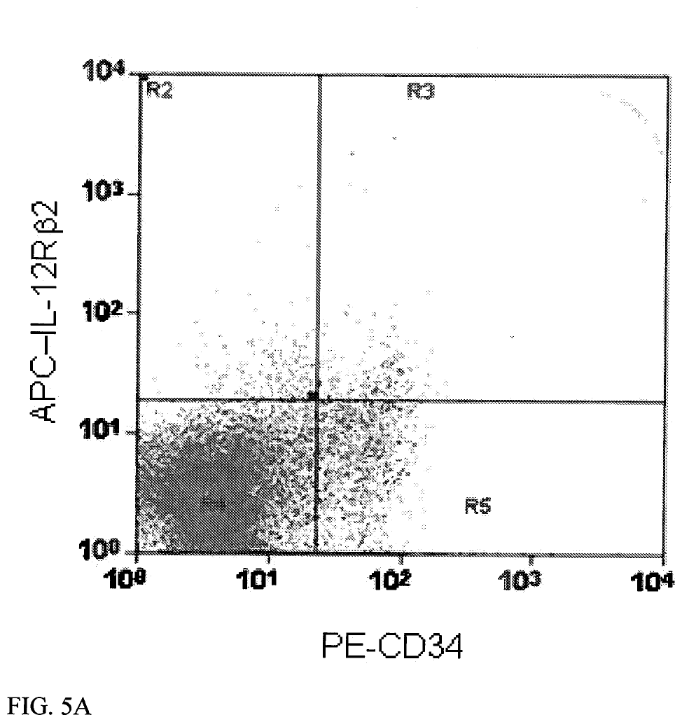

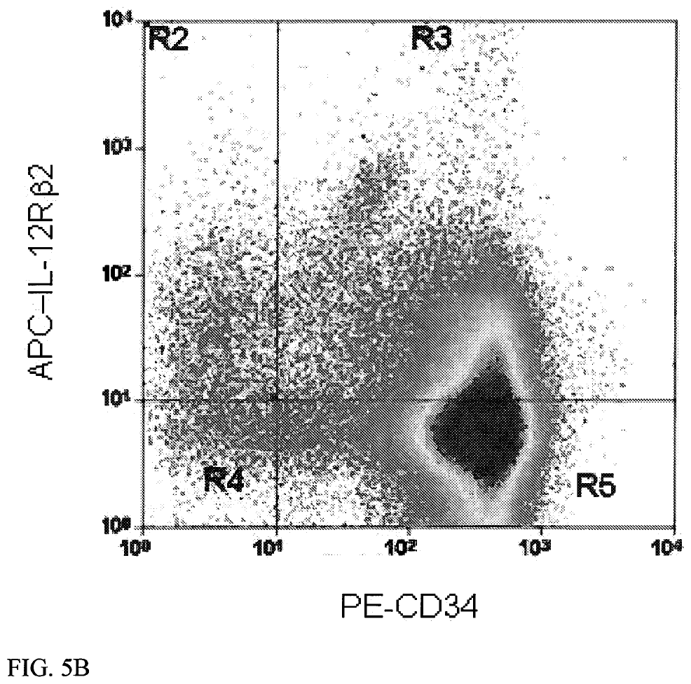

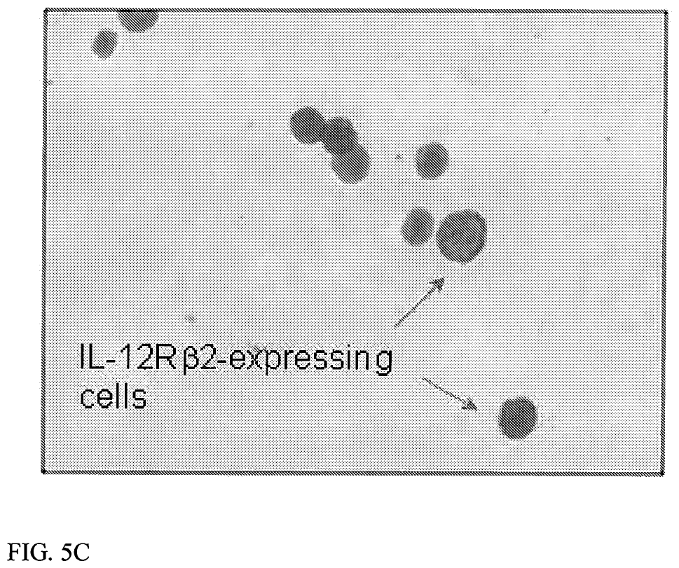

The hematopoietic regenerating function of rMuIL-12, the expression of IL 12Rβ2 on mouse Lin− cells and the proliferative effects of rMuIL-12 on mouse Lin−IL-12Rβ2+ cells suggest the potential impact of rHuIL-12 in regenerating human bone marrow. To evaluate the potential role of rHuIL-12 in human transplantation, we elucidated the expression of IL-12Rβ2 on human bone marrow hematopoietic stem/progenitor cells. We assessed the expression of IL-12Rβ2 on human bone marrow cells by flow cytometry analysis of Lin− cells and CD34+ cells labeled with antibodies against IL-12Rβ2 and CD34. CD34 cells were chosen as they have long been the accepted marker of choice for purification of hematopoietic stem cells for transplantation in the clinical setting. Among Lin− cells, approximately 0.5% to 4% of cells expressed IL-12Rβ2+ (Mean±SD: 1.89±1.21), regardless of the CD34 expression status (FIG. 5a). Among CD34+ cells, however, the expression of IL-12Rβ2 was highly donor dependent and detected on 6% to 57% of cells (Mean±SD: 19.59±17.52) (FIG. 5b). The presence of IL-12Rβ2 on CD34+ cells as detected by flow cytometry analysis was further demonstrated by immunocytochemical staining of human bone marrow CD34+ cells using a different antibody against IL-12Rβ2 (FIG. 5c). A negative control prepared with the same reagents but without incubation with the primary antibody against IL-12Rβ2 yielded no staining (data not shown).

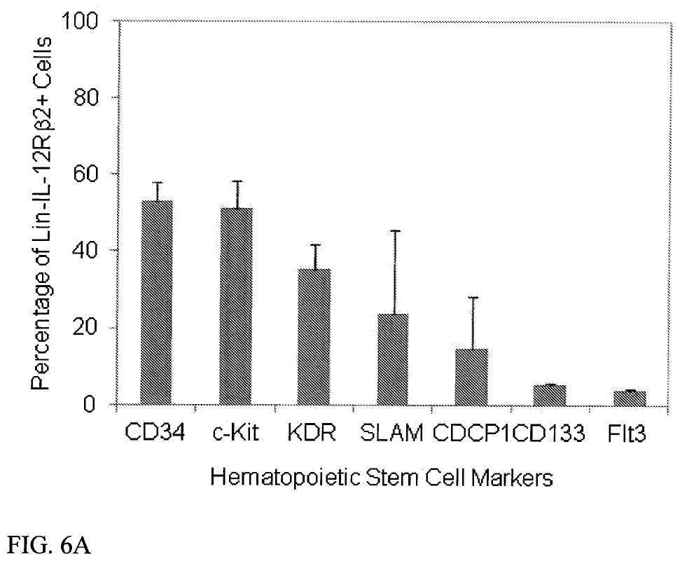

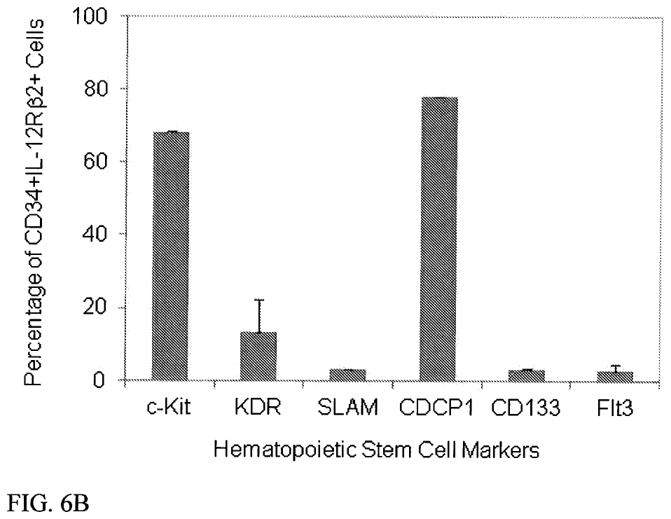

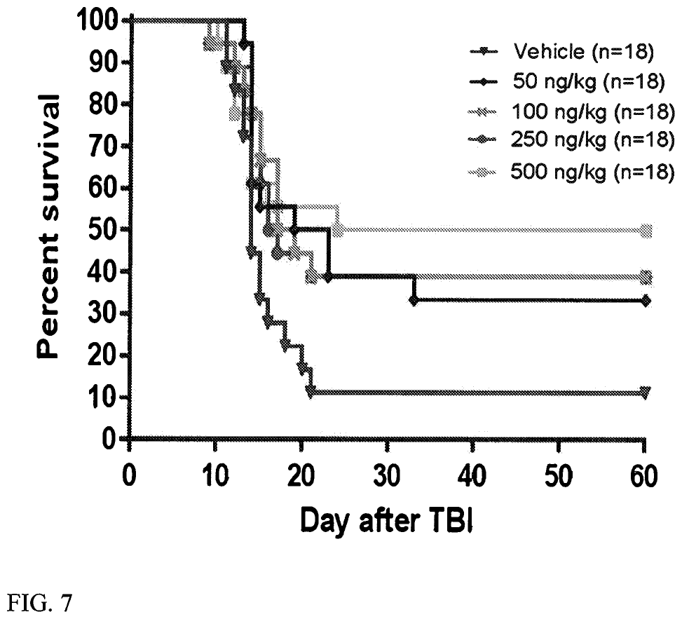

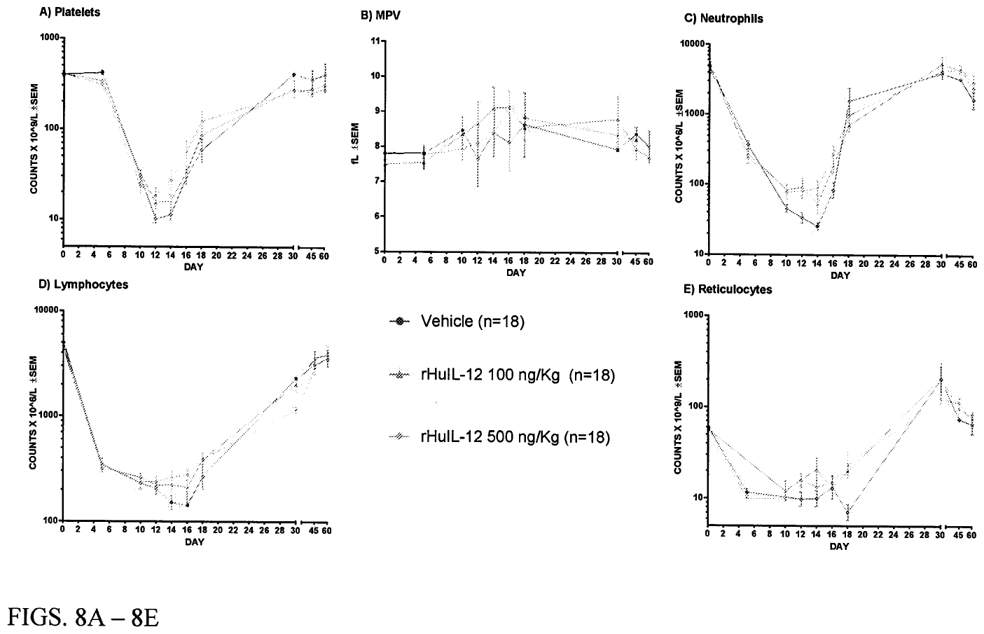

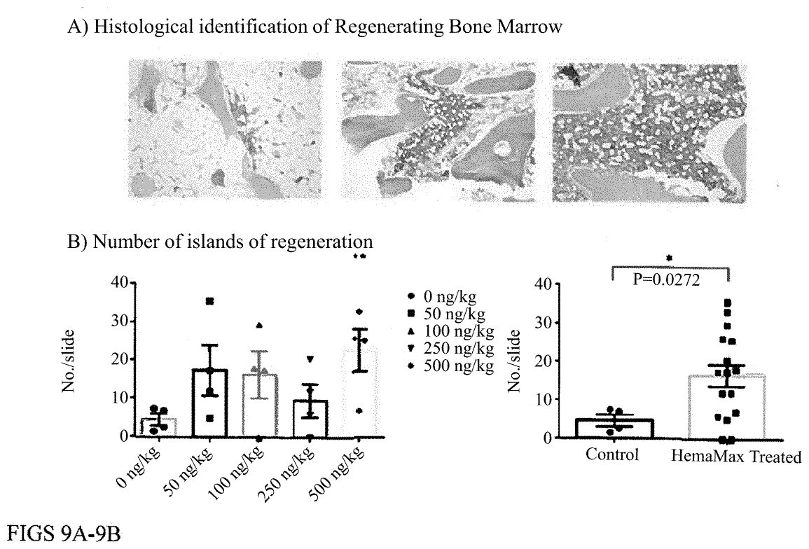

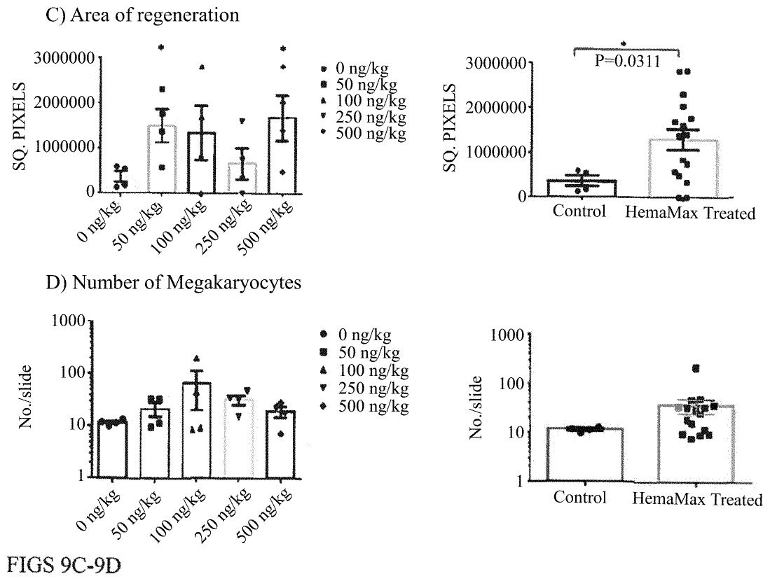

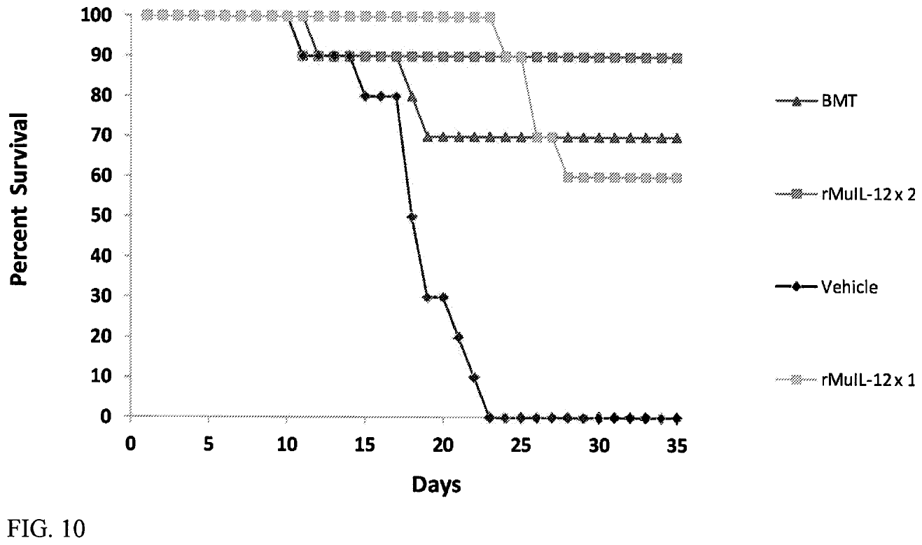

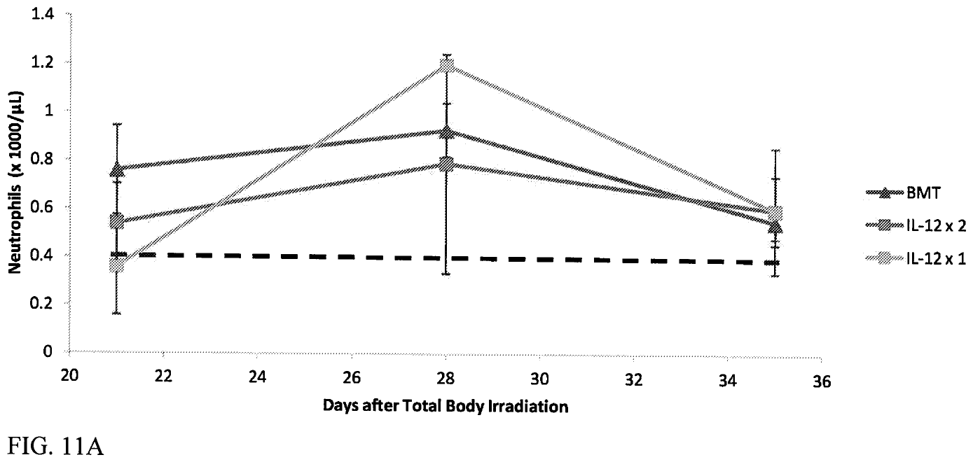

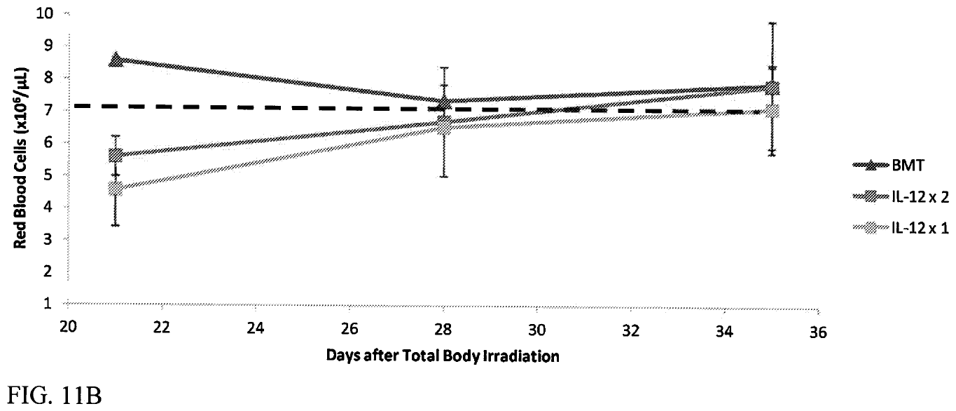

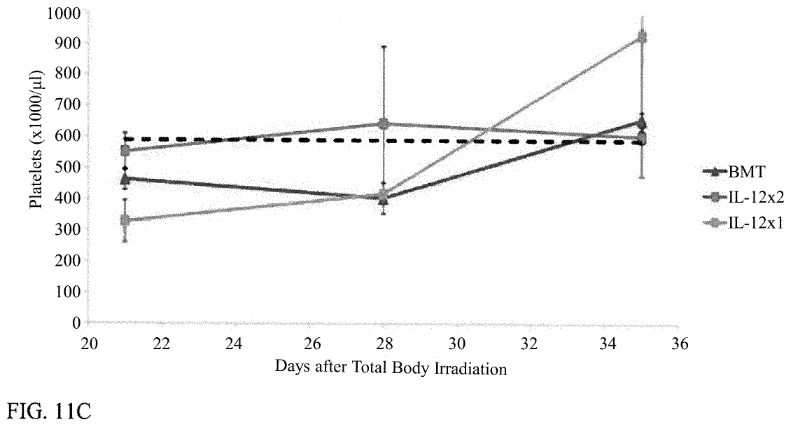

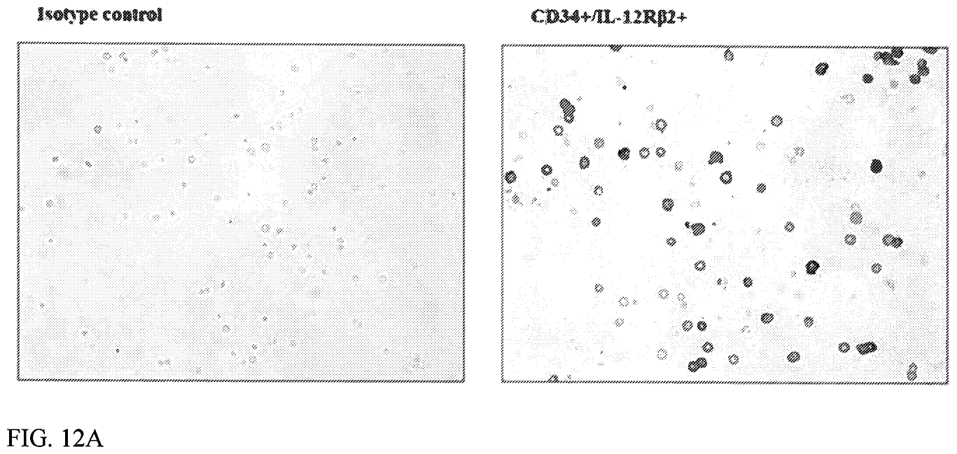

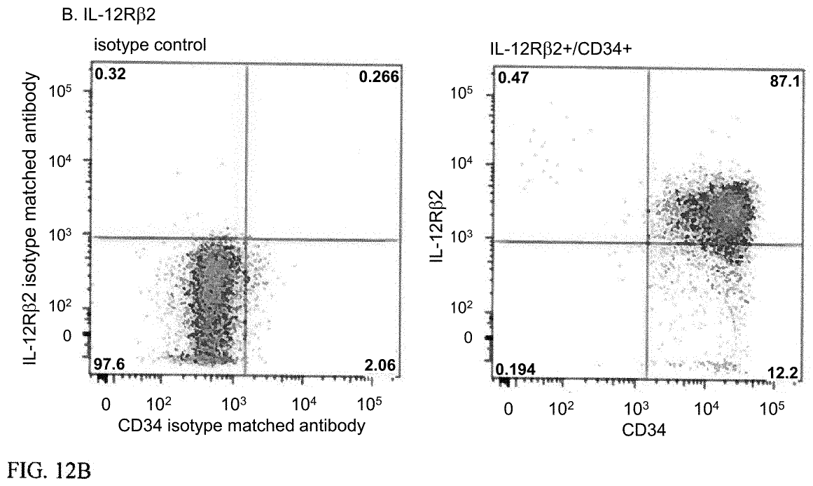

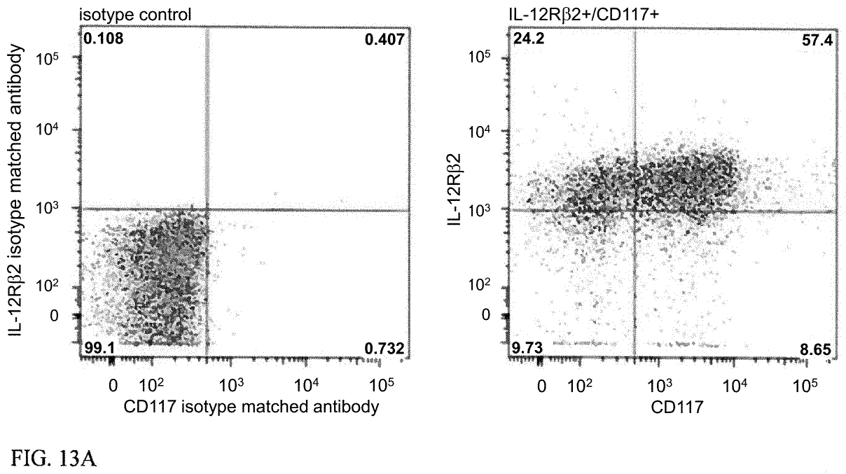

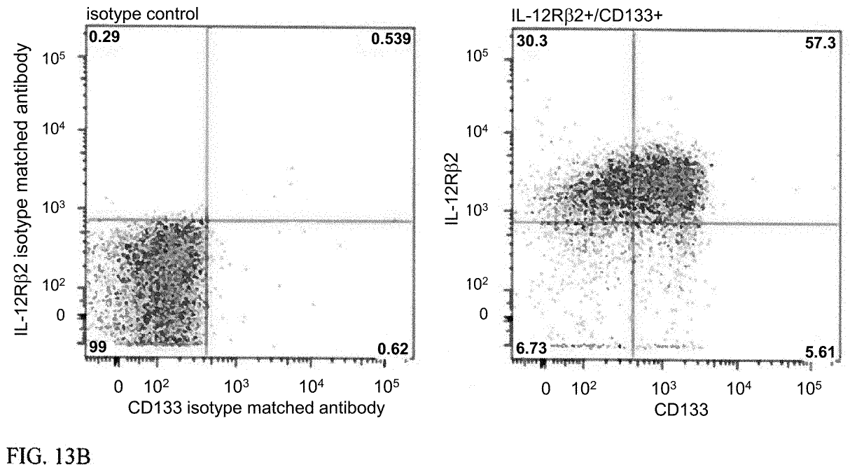

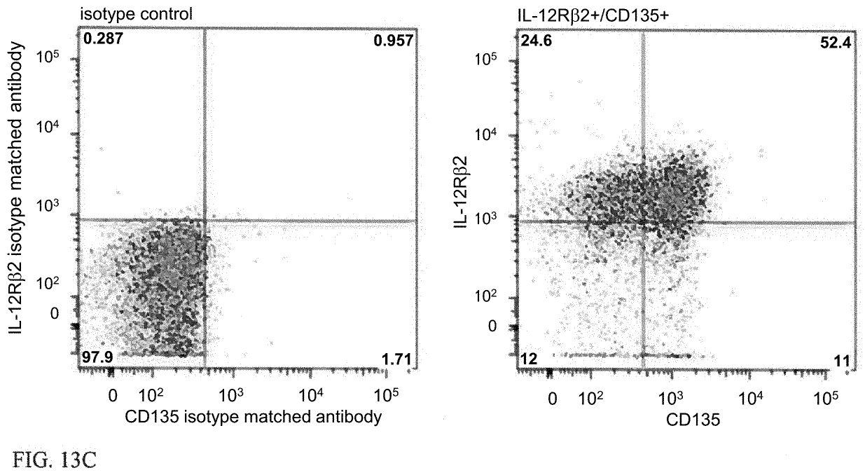

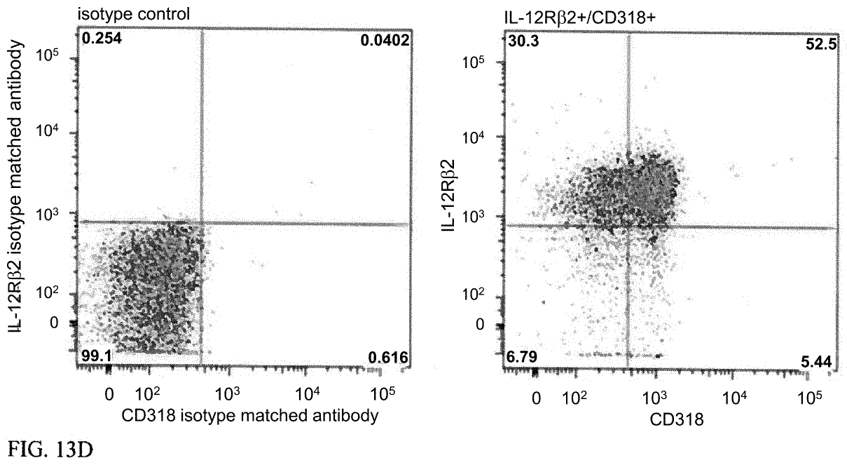

Co-expression of IL-12Rβ2 with other cell surface markers of HSCs was also evaluated by flow cytometry analysis in human bone marrow. Lin− cells and CD34+ cells were each co-labeled with antibodies against IL-12Rβ2 and CD34, c-kit, KDR, CD 133, Flt-3, CD 150 or CDCPI, which are known to be expressed on discrete population of primitive bone marrow cells. These analyses revealed that approximately 50% of Lin−IL-12Rβ2+ cells co-expressed CD34 (Mean±SD: 53±5) or c-Kit (Mean±SD: 51±7), 35% KDR (Mean±SD: 35±6), and 25% CD 150 (Mean±SD: 23±21) (FIG. 6a), Similar to Lin− cells, approximately 70% of CD34+IL-12Rβ2+ cells co-expressed c-kit (Mean±SD: 68±0.2), 80% CDCP I, and 15% KDR (Mean±SD: 13±9) (FIG. 6b).

The examples provided herein clearly demonstrated that an exemplary IL-12 preparation, HemaMax IL-12 effectively improved hematopoietic recovery following HSCT transplantation.

Discussion

HSCTs are used to treat cancer patients following myeloablative chemotherapy and/or radiotherapy for various hematological malignancies. Primary factors affecting the success of HSCT engraftment are the type and stage of cancer, preparative regimen for transplantation, quality and quantity of CD34+ cells, and post-transplant use of growth factors. (Ninan M J, et al., “Posttransplant Thrombopoiesis Predicts Survival in Patients Undergoing Autologous Hematopoietic Progenitor Cell Transplantation,” Biol Blood Marrow Transplant 2007; 13:895-904; Bensinger W I, et al. “Peripheral Blood Stem Cells (PBSCs) Collected After Recombinant Granulocyte Colony Stimulating Factor (rhG-CSF): an Analysis of Factors Correlating with the Tempo of Engraftment After Transplantation,” Br J Haematol 1994; 87:825-831; Klumpp T R, et al. “Phase II Study of High-Dose Cyclophosphamide, Etoposide, and Carboplatin (CEC) Followed by Autologous Hematopoietic Stem Cell Rescue In Women With Metastatic or High-Risk Non-Metastatic Breast Cancer: Multivariate Analysis of Factors Affecting Survival and Engraftment,” Bone Marrow Transplant 1997; 20:273-281.) Administrations of myeloid and erythroid growth factors are used to treat posttransplant neutropenia and anemia. (Held T K, et al. “Pharmacodynamic Effects of Haematopoietic Cytokines: the View of a Clinical Oncologist,” Basic Clin Pharmacol Toxicol 2010; 106:210-2 14.) However, such agents do not affect platelet regeneration. To overcome thrombocytopenia, patients rely on early post-transplant platelet transfusions. (Wandt H, et al., “New Strategies for Prophylactic Platelet Transfusion in Patients with Hematologic Diseases,” Oncologist 2001; 6:446-450.) As many as 17% of cancer patients after an initial recovery may experience a subsequent secondary post-transplant thrombocytopenia, which is significantly associated with a higher rate of death. (Ninan, M J, et al., supra. at 895-904.) Idiopathic post-transplant thrombocytopenia and low platelet counts are associated with poor engraftment with short and long term repopulating CD34+ cells and portend an increased risk of death from disease relapse. (Id.) Thus, there is a need for drugs that could improve the number of CD34+ cells for engraftment and increase hematopoietic recovery following transplantation. Currently, there are also no agents available to alleviate post-transplant thrombocytopenia, which is predictive of decreased overall survival.

rHuIL-12 in non-human primates, or rMuIL-12 in mice, has been shown to increase survival in models of TBI. (Basile L A, and Ellefson D, et al. “HemaMax™, a Recombinant Human Interleukin-12, is a Potent Mitigator of Acute Radiation Injury in Mice and Non-Human Primates, PLoS ONE,” Submitted 2011; Basile L A, and Gallaher T K, et al. “Multilineage Hematopoietic Recovery with Concomitant Antitumor Effects Using Low Dose Interleukin-12 in Myelosuppressed Tumor-Bearing Mice,” J Transl Med 2008; 6:26; Chen T, et al., “IL-12 Facilitates Both the Recovery of Endogenous Hematopoiesis and the Engraftment of Stem Cells after Ionizing Radiation,” Exp Hematol 2007; 35:203-213.) Several lines of evidence indicate that stimulation of bone marrow hematopoiesis may play a key role in pro-survival activity of rHuIL-12. (Basile L A, and Ellefson D, et al., supra.) When tested in vivo, rMuIL-12 increased peripheral blood cell recovery and survival in lethally irradiated mice similarly to a BMCT. Interestingly, recovery of platelets appeared earlier in the IL-12 treated mice than mice administered a BMCT. These findings are consistent with our previous reports demonstrating the hematopoiesis-promoting and pro-survival activities of rMuIL-12 and rHuIL-12 in mice and rhesus monkeys, respectively. (Basile L A and Ellefson D, et al., supra.; Basile L A and Gallaher T K, et al. supra., Chen T., et al., supra.) These hematopoietic recovery effects were also observed in tumor-bearing mice. In both Lewis lung and EL4 lymphoma models, rMuIL-12 provided early neutrophil, red blood cell and platelet recovery following sublethal TBI. Further, IL-12 decreased tumor burden synergistically with radiation or chemotherapy in both lymphoma and lung cancer murine models. (Basile L A and Gallaher T K, et al. supra.) Consistent with our findings, administration of rHuIL-12 given as immunotherapy induces in vivo expansion of major lymphocyte subsets following peripheral blood stem cell transplantation in patients with hematological malignancies. (Pelloso D, et al. “Immunological Consequences of Interleukin 12 Administration After Autologous Stem Cell Transplantation,” Clin Cancer Res 2004; 10:1935-1942.)

In the current study, of note was similar potency of rMuIL-12 and BMCT in increasing blood cell recovery and survival in lethally irradiated animals. Moreover, the hematopoiesis-promoting activity of rMu IL-12 appears to complement that of BMCT, as a combination of suboptimal rMuIL-12 dose with a low dose BMCT synergistically increased survival, while either of the treatments alone could not rescue lethally irradiated animals. (Chen T., et al., supra.) In the current study, platelet recovery was more robust in rMuIL-12 treated mice compared to BMCT. Comparable potencies and complementary activities of rMuIL-12 and BMCT suggest that rHuIL-12 could be an adjunct option to HSCT for enhancing engraftment potential and hematopoietic recovery of HSCs, particularly recovery of platelet counts.

The ability of rMuIL-12 to increase murine bone marrow hematopoiesis indicates the presence of functional IL-12 receptors (IL-12R) on primitive, extant hematopoietic cells, which when bound by IL-12, triggers events that initiate bone marrow regeneration. In support of this hypothesis, we demonstrated that rMuIL-12-treated murine bone marrow was characterized by the presence of IL-12Rβ2-expressing stem/progenitor cells, megakaryocytes, and osteoblasts, suggesting that both HSCs and niche cells may be direct targets of rHuIL-12. Activation of osteoblasts is crucial for the survival, expansion, and homing of hematopoietic stem cells and megakaryocytes. (Ahmed N, et al., supra, de Barros A P, et al., “Osteoblasts and Bone Marrow Mesenchymal Stromal Cells Control Hematopoietic Stem Cell Migration and Proliferation in 3D in vitro Model,” PLoS One 2010; 5:e9093; Hamada T, Mohle R, Hesselgesser J, et al. “Transendothelial Migration of Megakaryocytes in Response to Stromal Cell-Derived Factor 1 (SDF-1) Enhances Platelet Formation,” J Exp Med 1998; 188:539-548; Hodohara K, et al. “Stromal Cell-Derived Factor-1 (SDF-1) Acts Together with Thrombopoietin to Enhance the Development of Megakaryocytic Progenitor Cells,” (CFU-MK). Blood 2000; 95:769-775; Kiel M J, et al., “Maintaining Hematopoietic Stem Cells in the Vascular Niche,” Immunity 2006; 25:862-864; Wang J F, et al., “The Alpha-chemokine Receptor CXCR4 is Expressed on the Megakaryocytic Lineage from Progenitor to Platelets and Modulates Migration and Adhesion,” Blood 1998; 92:756-764.) It has been shown that exposure to lethal doses of radiation leads to osteoblastic niche expansion whereby the surviving pool of radioresistant osteoprogenitors proliferates close to the endosteal bone areas. (Dominici M, Rasini V, et al., supra.) In this study, surviving megakaryocytes were also observed close to the trabecular bone endosteal surface rather than in their normal parasinusoidal site. Megakaryocytes release factors that stimulate the expansion of osteoblastic niche. (Id.) Consistent with these findings, immunohistochemical examinations in our study revealed a cellular configuration in mice bone marrow, showing cellular islands consisting of osteoblastic niche, megakaryocytes, and hematopoietic stem cells close to the bone. Collectively, these findings suggest that rHuIL-12 may act directly via IL-12 receptor to orchestrate the activities of HSCs and niche cells and to stimulate hematopoiesis. In agreement with this notion, IL-12 has been reported to enhance colony formation of 5-fluorouracil-treated human peripheral blood CD34+ cells in the presence of accessory human CD34− cells. (Grafte S, et al. IL-12 “Indirectly Enhances Proliferation of 5-FU-Treated Human Hematopoietic Peripheral Blood CD34+ Cells” Am J Hematol 1998; 58:183-188.)

The presence of IL-12β2 expression in the bone marrow from irradiated mice treated with rMuIL-12, as opposed to its absence in the bone marrow from irradiated, untreated mice suggests that rMuIL-12 may upregulate IL-12 receptor expression in bone marrow and exert its hematopoiesis-promoting activity directly via IL-12 receptors on HSC and niche cells. Consistent with this hypothesis, treatment with exogenous rMuIL-12 in the absence of radiation yielded an increase in the relative number of Lin−IL-12Rβ2+ cells among lineage negative cells of the bone marrow compartment, and resulted in an average 4.5 fold enhancement of IL-12Rβ2+ cells for the two different time points at which bone marrow was harvested. These data are also consistent with our BrdU incorporation assay in the absence of radiation, which showed an increase in BrdU positive cells in whole bone marrow (16.5%) in IL-12-treated mice as compared with untreated mice (7.5%) 21 hours after treatment (data not shown). From these data, we conclude that the observed increase in isolated and selected lin− IL-12Rβ2+ cells from bone marrow following in-vivo treatment with the rMuIL-12 ligand results from direct HSC expansion via the IL-12 ligand/IL-12 receptor system, leading to an increase in the number of daughter HSC bearing the IL-12 receptor.

The instant disclosure is the first report demonstrating that IL-12Rβ2 is expressed on mouse and human Lin− cells and human CD34+ cells, two pools of bone marrow cells encompassing hematopoietic stem and progenitor cells. The expression of IL-12Rβ2 on human CD34+ cells was also confirmed by immunocytochemical staining. Overall, these studies demonstrated that 1-4% of human Lin− cells and 6-50% of human CD34+ cells expressed IL-12Rβ2. A considerable number of Lin−IL-12Rβ2+ cells (20% to 50%) and CD34+IL-12Rβ2+ cells (15% to 80%) also co-expressed other potential markers of HSCs primarily c-kit, KDR, CD150, or CDCP1 (Hawley R G, et al., “Hematopoietic Stem Cells,” Methods Enzymol 2006; 419:149-179; Conze T, et al. “CDCP1 is a Novel Marker for Hematopoietic Stem Cells,” Ann N Y Acad Sci 2003; 996:222-226; Drake A C, et al. “Human CD34+ CD133+ Hematopoietic Stem Cells Cultured with Growth Factors Including Angpt15 Efficiently Engraft Adult NOD-SCID I12rgamma−/− (NSG) Mice,” PLoS One 2011; 6:e18382; Ziegler B L, et al. “KDR Receptor: a Key Marker Defining Hematopoietic Stem Cells,” Science 1999; 285:1553-1558.) Of note was c-Kit, a receptor for stem cell factor (SCF) whose hematopoiesis-stimulating activity is dramatically (7-fold) increased by rMuIL-12 in mice in vitro. (Jacobsen S E, et al., “Cytotoxic Lymphocyte Maturation Factor (Interleukin 12) is a Synergistic Growth Factor for Hematopoietic Stem Cells,” J Exp Med 1993; 178:413-418.) The receptor c-kit has been shown to play an important role in self-renewal and maintenance of HSCs in vivo. (Porrata L F, et al., “Early Lymphocyte Recovery Predicts Superior Survival After Autologous Hematopoietic Stem Cell Transplantation in Multiple Myeloma or Non-Hodgkin Lymphoma,” Blood 2001; 98:579-585.) The c-kit/SCF complex plays an important role in stem cell adhesion to its microenvironment in the bone marrow and HSC homing. IL-12 and SCF have been shown to synergistically support proliferation of lymphoid primitive hematopoietic progenitors in vitro. (Hirayama F, et al., “Synergistic Interaction Between Interleukin-12 and Steel Factor in Support of Proliferation of Murine Lymphohematopoietic Progenitors in Culture,” Blood 1994; 83:92-98.) These unexpected and surprising synergistic effects and co-expression of IL-12Rβ2 with c-kit further demonstrated the role for IL-12 in hematopoietic regeneration and recovery. Consistent with prior reports, CD150 expression was mostly restricted to Lin− cells while CDCP1 expression was mainly limited to CD34+ cells Conze T, Lammers. (Conze T, Lammers, et al. supra; Sintes J, et al., “Differential Expression of CD150 (SLAM) Family Receptors by Human Hematopoietic Stem and Progenitor Cells,” Exp Hematol 2008; 36:1199-1204.) Lin− cells expressing CD150 have been shown to represent a subset of long-term reconstituting HSCs. (Buhring H J, et al., “CDCP1 Identifies a Broad Spectrum of Normal and Malignant Stem/Progenitor Cell subsets of Hematopoietic and Nonhematopoietic Origin,” Stem Cells 2004; 22:334-343.) Stimulation of CD34+ cells with CDCP1 reactive monoclonal antibody resulted in an increase in erythroid colony forming units. CDCP1 expression was also observed on cell types resembling mesenchymal cells. (Id.) Both Lin− cells and CD34+ cells expressed KDR, consistent with previous reports. (Ziegler B L, et al., supra.) The HSC niche has been shown to consist of mesenchymal stem cells characterized by a CD34−KDR+ phenotype, known to play a role in tissue regeneration and subsequently, KDR+ cells have been shown to possess reconstituting function. Co-expression of IL-12Rβ2 with KDR further suggests that IL-12Rβ2, expressed on HSCs or bone marrow niche cells may play an important role in hematopoietic recovery. Collectively, these findings suggest that the IL-12/IL-12 receptor pathway is implicated in human bone marrow hematopoiesis and that IL-12Rβ2 may represent a marker delineating a novel pool of hematopoietic stem and progenitor cells and bone marrow niche cells that can be exogenously stimulated by rHuIL-12 to expand and thereby reconstitute hematopoietic tissue following myeloablation.

The efficacy of IL-12 has been the subject of several studies in the context of its anti-tumor activity in cancer patients. In spite of intensive interest, IL-12 never advanced as a drug and never received Food and Drug Administration (FDA) approval because of its modest clinical activity and significant toxicity at high (300 ng/Kg to 600 ng/Kg), repeated dosing regimens (5 doses per week in a repeated regimen). (Atkins M B, et al., “Phase I Evaluation of Intravenous Recombinant Human Interleukin 12 in Patients with Advanced Malignancies,” Clin Cancer Res 1997; 3:409-417; Bajetta E, Del V M, Mortarini R, et al., Pilot Study of Subcutaneous Recombinant Human Interleukin 12 in Metastatic Melanoma,” Clin Cancer Res 1998; 4:75-85; Gollob J A, et al., “Phase I Trial of Twice-Weekly Intravenous Interleukin 12 in Patients with Metastatic Renal Cell Cancer or Malignant Melanoma: Ability to Maintain IFN-Gamma Induction Is Associated With Clinical Response,” Clin Cancer Res 2000; 6:1678-1692; Gollob J A, et al., “Phase I Trial of Concurrent Twice-Weekly Recombinant Human Interleukin-12 Plus Low-Dose IL-2 in Patients with Melanoma or Renal Cell Carcinoma,” J Clin Oncol 2003; 21:2564-2573; Lenzi R, et al., “Phase I Study of Intraperitoneal Recombinant Human Interleukin 12 in Patients with Mullerian Carcinoma, Gastrointestinal Primary Malignancies, and Mesothelioma,” Clin Cancer Res 2002; 8:3686-3695; Little R F, et al., “Phase 2 Study of Pegylated Liposomal Doxorubicin in Combination with Interleukin-12 for AIDS-Related Kaposi Sarcoma,” Blood 2007; 110:4165-4171; Motzer R J, et al., “Phase I Trial of Subcutaneous Recombinant Human Interleukin-12 in Patients with Advanced Renal Cell Carcinoma,” Clin Cancer Res 1998; 4:1183-1191; Ohno R, et al., “A Dose-Escalation and Pharmacokinetic Study of Subcutaneously Administered Recombinant Human Interleukin 12 and its Biological Effects in Japanese Patients with Advanced Malignancies,” Clin Cancer Res 2000; 6:2661-2669; Portielje J E, et al., “Phase I Study of Subcutaneously Administered Recombinant Humanlinterleukin 12 in Patients with Advanced Renal Cell Cancer,” Clin Cancer Res 1999; 5:3983-3989; Robertson M J, et al., “Interleukin 12 immunotherapy After Autologous Stem Cell Transplantation for Hematological Malignancies,” Clin Cancer Res 2002; 8:3383-3393; van Herpen C M, et al., “Intratumoral Administration of Recombinant Human Interleukin 12 in Head and Neck Squamous Cell Carcinoma Patients Elicits a T-Helper 1 Profile in the Locoregional Lymph Nodes,” Clin Cancer Res 2004; 10:2626-2635.) At relatively lower doses, therapy with exogenous IL-12 was well tolerated in patients with hematological malignancies at doses ranging from 30 ng/Kg to 250 ng/Kg given once approximately 2 months after peripheral blood stem cell transplantation followed 2 weeks later by 5 daily administrations in a repeated regimen. (Robertson M J, et al., supra.)

In contrast, we have found that rHuIL-12 exerts its hematopoietic-promoting activity at low nanogram per kilogram doses in animals which are equivalent to less than 100 ng/Kg as a human dose, given only once or twice in both our murine and rhesus monkey studies. Moreover, in our toxicology studies, our proprietary rHuIL-12 was well tolerated in rhesus monkeys after up to seven doses of 1000 ng/Kg (data not shown), which is equivalent to a human dose of about 300 ng/kg, with no overt sign of toxicity. These findings indicate that the rHuIL-12 dose for hematopoietic regeneration will be substantially lower than the IL-12 doses previously used in cancer patients, suggesting a more favorable safety profile for rHuIL-12 in HSC transplantation patients.

Our findings demonstrate that rHuIL-12 may potentially offer additional therapeutic value by stimulating multilineage recovery of peripheral blood cell counts, particularly platelets. The anti-tumor and immune stimulating effects of IL-12 further add to its therapeutic value in cancer patients. IL-12/IL-12R thus represents a viable pathway that can be introduced via administration of exogenous IL-12 to increase remission in patients undergoing HSC transplantation.

### CONCLUSIONS

Exemplary methods and compositions comprising rMuIL-12 promoted hematopoiesis and increased the recovery of peripheral blood cells and survival in lethally irradiated mice as effectively as a BMCT, indicating that rHuIL-12 therapy can to increase HSC engraftment following HSCT. We identified IL-12Rβ2 expressing cells in irradiated mouse bone marrow which are potential targets of IL-12. Administration of rMuIL-12 increased the number of IL-12R 2 expressing Lin− cells in mouse bone marrow, indicating that bone marrow HSCs and niche cells are the direct target of rMuIL-12 and that hematopoiesis-promoting activity of rMuIL-12 is mediated by IL-12 receptors on HSCs. Finally, we show expression of IL-12Rβ2 on human bone marrow lin− and CD34+ cells, indicating a potential role for IL-12 in human transplantation.

### Example

TABLE 1
Percentage of Bone Marrow Lin− IL-12Rβ2+ Cells
in Mouse Treated With Vehicle or rMuIL-12. Exogenous IL-12
increases the relative number of IL-12Rβ2+ cells
among lineage negative cells of the bone marrow compartment,
and resulted in an average 4.5 fold enhancement of IL- 12Rβ2+ cells.

| | | | Fold |
|---|---|---|---|
| Percentage of IL-12Rβ2+ | Vehicle | rMuIL-12 | increase |
| Experiment 1 (after 21 | 0.6 | 2.1 | 3.5 |
| hours) | | | |
| Experiment 2 (after 25 | 0.5 | 2.7 | 5.4 |
| hours) | | | |

### Example 11

Demonstration of Efficacy: A Single, Low Dose rHuIL-12 Restores Hematopoiesis and Increases Survival in Rhesus Monkeys Exposed to Lethal Radiation

A single, low-dose of rHUIL-12 significantly increased survival relative to placebo in non-human primates exposed to lethal radiation as a single agent, without the use of supportive care rHUIL-12 reduced rates of sepsis and severe neutropenia/thrombocytopenia and increased bone marrow regeneration relative to placebo.

The hematopoietic syndrome of the acute radiation syndrome (HSARS) leads to death in humans exposed to lethal total body irradiation (TBI). Recombinant human interleukin-12 (rHuIL-12) is being developed for mitigation of HSARS under the FDA Animal Rule, where efficacy is proven in an appropriate animal model (e.g, non-human primates) and safety is demonstrated in humans. Rhesus monkeys (9 animals/sex/dose group) were randomized to receive a single subcutaneous injection of rHuIL-12 (0 [placebo], 50, 100, 250, or 500 ng/kg), without antibiotics, fluids or blood transfusions, 24-25 hours after TBI (700 cGy). Survival rates at Day 60 were 11%, 33%, 39%, 39%, and 50% for the placebo, 50, 100, 250, and 500 ng/kg rHuIL-12 dose groups, respectively (log rank p<0.05 for each dose vs. placebo). rHuIL 12 also significantly reduced the incidence of severe neutropenia, severe thrombocytopenia, and sepsis. Additionally, bone marrow regeneration following TBI was significantly greater in monkeys treated with rHuIL-12 than in controls. These data demonstrate that a single injection of rHuIL-12 delivered one day after TBI can significantly increase survival and significantly reduce radiation-induced hematopoietic toxicity and infections. Therefore, rHuIL-12 is efficacious as an effective stand-alone medical countermeasure against the lethal effects of radiation exposure.

Acute radiation syndrome (ARS) is a life-threatening illness caused by whole body or significant partial-body exposure to radiation doses>1 Gy over a short period of time, as would occur in the event of a nuclear accident or attack. The pathophysiology of ARS is well understood, and is similar across all mammals, involving detrimental effects on the hematopoietic, gastrointestinal, central nervous and cutaneous systems. In the hematopoietic subsyndrome of ARS (HSARS), toxicity is due to rapid bone marrow ablation, leading to pancytopenia. HSARS ultimately results in death due to infection and/or hemorrhage over the range of 2 weeks to 2 months, depending on the radiation exposure level.

While the availability of a radiation medical countermeasure (R-MCM) in the event of a large scale radiation emergency is critical for saving lives, no treatments are currently approved by the US Food and Drug Administration (FDA). Typical treatment guidelines for HSARS include short- or long-term cytokine administration, depending on the exposure level. While available cytokine products support the growth of some individual cell types (such as G-CSF for neutrophils) 1, reviews of their use have not shown consistent reductions in overall mortality after TBI, and they cytokines are not approved by FDA for the HSARS indication. An optimal R-MCM against HSARS resulting from a nuclear disaster or accident would be able to regenerate all the lineages of the bone marrow compartment, and, given the expected logistic impediments, should be effective when administered hours to days after exposure, preferably as a single dose, and in the absence of intensive supportive care. These requirements are not fulfilled by G-CSF, which affect only granulopoietic lineage, require multiple daily administration and have been shown to improve survival only in combination with intensive supportive care. 9 Additionally, G-CSF in the context of radiation exposure was reported associated with long-standing isolated thrombocytopenia10, and even delayed adverse effects involving lung toxicity 18 and fibrosis (Aeolus reference).

We previously reported that a single administration of recombinant human IL-12 (rHuIL-12 given 24-25 hours after irradiation, in the absence of antibiotics, fluids or blood products, improved survival in both a murine HSARS model and in a proof-of-concept, open-label, male-only study of non-human primates (NHP).8 These findings supported the further development of rHuIL-12 as an R-MCM for HSARS under the FDA Animal Rule, where efficacy is proven in an appropriate animal model (eg, non-human primates) and safety is demonstrated in humans. Herein, we describe results of a randomized, blinded, pivotal phase efficacy study of the radiation countermeasure effects of rHuIL-12 in a large group of male and female rhesus monkeys. This Phase 2 study advances rHuIL-12 towards approval under the Animal Rule.

Methods

Animals

Rhesus monkeys (Macaca mulatta) were obtained from the Yongfu County Xingui Wild Animals Raising Ltd., China. Monkeys (3 to 5 years old, and 3.0 to 5.7 kg at the start of treatment) were housed individually and acclimated for ≥5 weeks prior to irradiation. Harlan Teklad Certified Hi-Fiber Primate Diet #7195C (Harlan Laboratories, Indianapolis, Ind.) was provided twice daily.

Randomization and Blinding

In the main study, male and female animals (45 each; 9 animals per sex per dose group) were randomized, stratified by body weight, to the following doses of rHuIL-12: vehicle; 50 ng/kg, 100 ng/kg, 250 ng/kg, or 500 ng/kg administered by SC injection (groups 1-5). The pathologist and study staff other than the study team leader and those involved with irradiation were blinded. An evaluation of the bone marrow at Day 12 was conducted in a blinded manner using separate animals randomized to the same doses of rHuIL-12 (2 per sex per group).

Total Body Irradiation

The dose of 700 cGy (60 cGy/minute from a Theratron 1000 Co60 source [Best Theratronics; Ottawa, Ontario, Canada]) was based on available historical data from CiToxLAB North America. TBI was conducted with animals in a vertical position, as described previously. For homogenous dose distribution, the first half-dose was delivered anteroposterior and the second half-dose was delivered posteroanterior. Dosimetry was verified to be within 10% of prescribed dose using nanodot chips (Landauer, Inc., Glenwood, Ill., USA) positioned on the front and back of each animal.

rHuIL-12 Dosing

Clinical grade rHuIL-12 or vehicle was administered by SC injection between the scapulas approximately 24-25 hours following TBI. The concentration of the test item in each dosing sample was verified by intertek Pharmaceutical Services (San Diego, Calif.) using the Quantikine® Human IL-12 ELISA Kit (R&D Systems Inc., Minneapolis, USA).

Assessments

Clinical signs were recorded twice daily. Decreases in appetite (based on food intake) and physical activity were recorded daily and scored as flows: 1=slight; 2=moderate; and 3=severe. A detailed physical examination was performed prior to rHuIL-12 dosing and twice weekly thereafter. Body temperature (auricular) was taken prior to irradiation and on Days 3-10, 12, 14, 16, 18, 30, 45, and 60, or when clinically justified. Blood sampling (0.5 mL for peripheral blood counts was performed prior to irradiation and at Days 5, 10, 12, 14, 16, 18, 30, 45, and 60. Blood was collected for hemoculture in cases of febrile neutropenia (absolute neutrophil count<0.05 109/L together with rectal body temperature≥104° F./40.0° C.) and at necropsy.

Terminal Procedure

Animals were euthanized prior to Day 60 if any of the following criteria were observed: respiratory distress; complete anorexia for 3 day; loss of >20% of initial body weight over a 3 day period; severely decreased activity level (recumbent during an entire observation period or unresponsiveness to touch); acute loss of >20% estimated blood volume; generalized seizure activity; abnormal appearance (posture, rough coat, head down, exudates around eyes and nose, pallor, tucked abdomen and clinical appearance) associated with abnormal vital signs: severe dehydration with hypothermia (decreasing rectal temperature reaching <34.6° C. and severely decreased activity level) or hyperthermia (temperature>40.1° C. and severely decreased activity level). Euthanasia decisions were made by a team of technicians and veterinarians blinded to the animal group assignment. Surviving animals were euthanized at Day 60 following TBI.

Necropsy comprised an external macroscopic examination, a detailed internal examination, evaluation of organ weights and gross pathology, and collection of tissues for histopathology. Presence of hemorrhage was scored for major organs as follows: 0=absences; 1=minimal; 2=slight; 3=moderate; 4=marked; 5=severe. For histological examination, tissues were embedded in paraffin, sectioned and stained with hematoxylin and eosin-phloxin (H & E).

Microbiological analysis was conducted on brain, heart, kidney, liver, both lungs, and spleen. Bacterial growth was scored (0 to 4) for each organ. The total score was summed for each animal; the mean score was calculated score for each treatment group.

Bone Marrow Histopathology

Animals in the bone marrow evaluation group were to be euthanized on Day 12 after TBI. One animal underwent unscheduled euthanasia on Day 11; all animals were included in the analysis. Two H&E sections for each animal were scanned on an Olympus BX41 compound microscope. Images of approximately 40 fields of view encompassing each femur section in its entirety were acquired on Infinity Analyze software v5.0 at a magnification of 10×. The number of bone marrow regeneration islands was determined by visual quantification in each field of view in each section. The total area of bone marrow regeneration was determined using ImageJ software, version 1.46. The mean number of regeneration islands and the mean area of regeneration from two sections per animal were used in the statistical analyses. The number of megakaryocytes was determined visually in each femur section.

Statistical Analysis

All statistical comparisons were conducted for according to sex and for the entire study population. Survival functions were estimated using the Kaplan-Meier product-limit method applied on daily intervals. The control group was compared to each of the other treated groups using the Mantel log-rank test. GLP analysis was performed at CiToxLAB.

Group comparisons for incidences of severe neutropenia (defined as neutrophil count<0.05×109/L), severe thrombocytopenia (defined as platelet count<10×109/L), and hemoculture positivity were performed using Fisher Exact test. If the overall comparison was significant (p<0.05), pair-wise comparisons between the control group and each of the dose groups was done using the Fisher Exact test.

Group means for bone marrow regeneration data (number and area of regeneration islands) were compared by a one-tailed t-test using the statistical software program Prism version 6 (GraphPad, San Diego, Calif.). Differences with p<0.05 were considered significant.

Results

Survival

Survival data is present in FIG. 1. The administered radiation dose corresponded to an approximate LD90/60 (2/18 animals survived in the untreated groups) under the conditions of this experiment (no antibiotics, fluids or blood transfusions). Fifty-eight out of 59 deaths occurred between Day 9 and Day 24, which is consistent with previously observed rates and timing of death due to HSARS in rhesus monkeys, and one death occurred at Day 33. The highest proportion of deaths occurred between Day 11 and Day 21 with Day 14 being the peak day of death for both control and rHuIL-12-treated groups. Death frequency was similar for males and females. All deaths, regardless of cause, were included in the statistical analysis of efficacy. Each rHuIL-12 treated group showed statistically significant increases in survival over the vehicle (log rank test p<0.05). In the vehicle group, 2 of 18 animals survived (11%, both males), while 33% (3 males and 3 females), 39% (4 males and 3 females), 39% (4 males and 3 females), and 50% (5 males and 4 females) survived in rHuIL-12 treated groups 2-5, respectively. Pair-wise comparison between rHuIL-12-treated groups showed no significant differences.

Possible cause of unscheduled death prior to Day 60 was predominantly infection. Also, macroscopic and microscopic evidence of hemorrhage was observed in a variety of organs.

Hematology

Blood counts for platelets, mean platelet volume, neutrophils, lymphocytes, and reticulocytes are presented in FIG. 2 A-E, respectively. Hematology measurements were made at a pre-radiation time point corresponding to basal levels, and thereafter, at post-irradiation time points of 5, 10, 12, 14, 16 and 18 days, which corresponds to the period of severe cytopenias seen in the HSARS model, and at 30, 45 and 60 days to evaluate the return to basal levels in surviving animals.

Platelets

Platelet nadirs occurred at day 12 or 14, depending on the dosing group. Significant thrombocytopenia (<50×109/L) was present over the 10-15 day interval. The nadir average of the control group was 10.1×109 platelets/L, which was lower than the nadir average for each of the treated groups, which were 12.1, 15.5, 12.7, and 18.6×109 platelets/L in rHuIL-12 treated groups 2-5, respectively. By day 18, initial recovery was observed in survivors in all groups, with full recovery on day 30. The proportions of blood samples with severe thrombocytopenia (platelets<10×109/L) between Day 10 and 18 were 33% in the control group and 34%, 20%, 22% and 12% in rHuIL-12 treated groups 2-5, respectively. The pair-wise comparison to control was significant for the 500 ng/kg group (Fisher exact test p=0.0073). Mean platelet volume was increased between day 14 and 18, corresponding to the release of young platelets from recovering bone marrow. Average peak values were 8.64 fL in the control group compared with 9.21, 9.13, 9.56 and 9.17 fL in the rHuIL-12 treated groups 2-5, respectively.

Neutrophils

Neutrophil nadirs occurred between days 10 and 14, depending on dosing group. Severe neutropenia (<50×106/L) occurred in 100% animals in the control group and in 88.9%, 77.8%, 83.3%, and 72.2% animals in the rHuIL-12 treated groups 2-5, respectively. The nadir average of the control group was 26×106/L, which was lower than the nadir average for each of the treated groups, which were 34, 54, 39, and 78×106/L in the groups treated with rHuIL-12 at doses of 50, 100, 250 and 500 ng/kg, respectively. Neutrophil recovery was initiated by day 18 and completed by day 30. The percentage of blood samples presenting with severe neutropenia on days 10 to 18 was 67% in the control group compared to 46%, 35%, 46% and 31% at doses of 50, 100, 250 and 500 ng/kg, respectively (p=0.0196, 0.0005, 0.0278, and <0.0001, Fisher exact test).

Lymphocytes

Lymphocyte nadirs occurred between days 10 and 16, depending on dosing group. All groups showed severe lymphopenia down to 7-10% of pre-radiation levels. The nadir average of the lymphocytes in the control group was 0.143×109/L, which was lower than the nadir average for each of the treated groups (0.163, 0.213, 0.220, 0.239×109/L at doses of 50, 100, 250 and 500 ng/kg, respectively). On Day 18, initial recovery from the nadir was observed in all groups. By day 30, group average levels still ranged from 30% to near 60% of the pre-radiation levels, while on days 45 and 60 the lymphocyte counts were in the normal range but still were slightly lower than the baseline levels. Statistical significance was not reached for males and females together, but the difference in the lymphocyte nadir between controls and animals treated with rHuIL-12 was statistically significant in females treated at the 50, 250, and 500 ng/kg dose levels (Sidak adjusted t-test p=0.0443, 0.0103, and 0.0211, respectively).

Red Blood Cells and Reticulocytes

The red blood cells nadir occurred on Day 18 in all groups, which represented a 37% reduction from baseline. Red blood cells nadirs were comparable in all groups (data not shown). Reticulocyte nadirs were 7.1×109/L in the control group and 8.7×109/L, 12.1×109/L, 9.1×109/L, and 9.8×109/L in the 50, 100, 250, and 500 ng/kg rHuIL-12 groups, respectively, suggesting a stimulatory effect of rHuIL-12 on erythropoiesis. However, the differences did not reach statistical significance.

Febrile Neutropenia

A total of 15 animals (8 males; 7 females) were reported to have febrile neutropenia, and most (10/15) were treated at the two highest dose levels of rHuIL-12. Ten of the 15 animals had a positive hemoculture and the most common bacteria were Escherichia coli and Staphylococcus aureus. Duration of febrile neutropenia was 1 day in all the animals and resulted in death on the same or next day in 12 animals. Three of the 15 animals survived to Day 60 (1 in the 250 ng/kg group and 2 in the 500 ng/kg group). Notably, 2 of the 3 surviving animals had negative blood cultures.

Bone Marrow

TBI-related bone marrow hypocellularity was observed in all animals that died before day 30. Bone marrow smears from these animals showed similar myelosuppressive effects in all treatment groups. For the survived animals the hypocellularity was completely reversed by Day 60 in all, except for one animal (500 ng/kg group) that had residual marked hematopoietic hypocellularity in the humerus bone marrow, but normal in other bone marrow sites.

In a companion study, a separate cohort of animals (2 per gender per group) was exposed to the same radiation level as in the survival study and treated with the same dose levels of rHuIL-12. All animals were sacrificed at Day 12, which represented the day of estimated maximal bone marrow suppression based on the timing of nadir of blood cell counts. Histological analysis of bone marrow showed severe hypocellularity with pockets of regeneration (FIG. 3A). Quantitation of the number of regeneration islands, total area of the regenerating islands, as well as number of megakaryocytes in each animal, was performed in a blinded analysis. Both the number and the area of the regenerative islands were higher in the rHuIL-12-treated groups compared to control, with all treated groups exhibiting a similar range of increased values (FIGS. 3B-C). The difference for both parameters reached statistical significance for 500 ng/kg groups, as well as in a pooled comparison (all treated groups versus control group) (p=0.0272 and p=0.0311, for the number and the area of islands of regeneration, respectively). Importantly, the megakaryocytes number was also higher for all rHuIL-12-treated groups relative to control, but the differences were not statistically significant (FIG. 3D).

Microbiology and Pathology

Infection

In the control group hemoculture positivity was 86%, compared to 65%, 65%, 47% and 44% in the rHuIL-12-treated groups 2-5, respectively. The difference was statistically significant for the two highest doses (p=0.0072 for each group). The decrease in the prevalence of infection was seen for both gram-negative and gram-positive bacteria. Bacteriological analysis of heart, kidney, liver, both lungs, brain and spleen was performed on necropsy for all animals. There was a decrease in mean bacterial growth score in rHuIL-12-treated animals compared to controls (Table 1).

Escherichia coli and Staphylococcus aureus were the most frequent isolates from organs and hemoculture. Twelve out of 16 control animals (75%) who were unscheduled euthanized were positive for E. coli in organ culture compared to 66.7%, 63.6%, 72.7% and 55.6% in rHuIL-12 treated groups 2-5, respectively. Ten out of 16 control animals (62.5%) who were unscheduled euthanized were positive for S. aureus in organ culture compared to 50.0%, 54.5%, 54.5% and 44.4% in rHuIL-12 treated groups 2-5, respectively.

Hemorrhage

Overall group mean hemorrhage scores for all organs, as well as a separate score for the gastrointestinal system, are shown in Table 1. Although the mean scores were higher in control group than in all rHuIL-12-treated groups, the differences did not reach statistical significance, likely due to substantial organ to organ and animal to animal variation. Notably, the proportions of animals that had hemorrhage scores≥4 in at least one organ were higher in the control group than in the groups treated with rHuIL-12, and brain hemorrhage was found only animals in the control group (2 animals).

Discussion

The rhesus monkey model used in this study is an established model of human HSARS, as the hematologic effects of TBI and the resulting occurrence of infection and hemorrhage in this model are similar to those reported for humans 9,10. The data from this randomized, blinded, placebo-controlled study demonstrate a positive and significant effect of a single, subcutaneous injection of rHuIL-12, over a 10-fold dose range, on survival following lethal TBI (700cGy; LD90/60) in this rhesus monkey model of HSARS. Notably, this effect was achieved without the use of supportive care. These results are consistent with beneficial effect of rHuIL-12 on survival found in our proof-of-concept study in NHP 8.

In the current study, clinical signs (vomiting, diarrhea, and body weight) resulting from the TBI generally were similar among all treatment groups and between the sexes (see Supplementary). Decreases in activity and appetite, which occurred during the period of blood cell count nadirs and highest rates of infection, hemorrhage and death, were greatest in the control group and smaller in groups treated with rHuIL-12.

Radiation-induced bone marrow suppression was mitigated by rHuIL-12: animal groups treated with rHuIL-12 showed statistically significant reductions in the occurrence of severe neutropenia and severe thrombocytopenia, as well as attenuated nadirs for lymphocytes, neutrophils, platelets, and reticulocytes. Mean platelet volume also was increased in animals treated with rHuIL-12 relative to controls, suggesting the release of newly formed platelets from the bone marrow. Quantitative analysis of the bone marrow regenerative pockets in a companion study supported the conclusion that rHuIL-12 alone has a stimulatory effect on hematopoiesis allowing for regeneration of bone marrow and recovery of the major blood cell components. Notably, the megakaryocytes number was also higher in rHuIL-12-treated groups relative to control. Stimulation of multiple hematopoietic lineages in vivo by rHuIL-12, as seen in this study, is consistent with previous reports, where IL-12 stimulated growth of hematopoietic stem cells and progenitors in vitro (4-6) and with our previous study in tumor-bearing mice. 11

Consistent with the reduction in severe neutropenia, the incidence of blood culture positivity for infection was significantly reduced from 86% in the control group to 47% and 44% in the groups that received rHuIL-12 at doses of 250 or 500 ng/kg, respectively. These data demonstrate that rHuIL-12 administered 24 hours after TBI decreased infectivity of broad-spectrum bacteria in the absence of antibiotics.

In support of our finding of a reduction of severe neutropenia and decrease in infections and sepsis, IL-12 is known to have multiple stimulatory effects on innate and adaptive immunity, which likely contributed to the decrease incidence of infection. At the early stages following irradiation, Th1 function is reduced due to the suppression of endogenous IL-12 secretion from antigen presenting cells. 12,13 IL-12 administered after irradiation promotes the proliferation and activation of the surviving NK cells, macrophages and dendritic cells. 14,15 The tri-directional cross talk between NK, macrophages and dendritic cells further promotes their maturation, leading to the restoration of Th1 function and the establishment of early immune competence following TBI. Further, continuous production of endogenous IL-12 from pathogen-activated dendritic cells serves as a positive feedback loop and plays a key role in sustaining the initial response to exogenous IL-12. 17 Taken together, these IL-12 generated immune-mediated effects can account in large part for the positive survival benefit observed in this study, which are likely derived from the anti-infectivity properties of IL-12.

Consistent with the reduction in severe thrombocytopenia, rHuIL-12 treatment in this study was associated with lower severity of hemorrhage for animals that died or were euthanized prior to the scheduled termination. In support of our finding of reduced severe thrombocytopenia and hemorrhage, we recently reported that in the bone marrow the expression of the β2 subunit of the IL-12 receptor (IL-12Rβ2), which is most specific to IL-12 signaling, was found on hematopoietic stem cells, megakaryocytes and osteoblasts. The presence of IL-12Rβ2 receptors on these key bone marrow cells suggest that through its receptors, rHuIL-12 may promote proliferation and differentiation of the surviving stem cells and megakaryocytes following radiation exposure, enhancing platelets regeneration and reducing severe thrombocytopenia. The ability of rHuIL-12 to facilitate regeneration of platelets may be of clinical importance in indications other than HSARS mitigation, as there is currently no available drug that can facilitate platelet recovery following myelosuppressive therapies in cancer patients.

While leucocyte growth factors are recommended for use in victims of radiation, they are not approved by FDA for this indication. There is only one published study that demonstrated improved survival in rHuG-CSF- vs control NHP 9 in combination with intensive, trigger-based medical management (antibiotics, intravenous blood product transfusions, intravenous fluid replacement). We recently completed a randomized blinded study comparing single injection of rHuIL-12 or vehicle with 18 injections of rHuG-CSF in the NHP model without supportive care. Preliminary analysis confirmed superior survival in rHuIL-12-treated group vs vehicle and vs G-CSF-treated group, while G-CSF did not increase survival compared to vehicle (manuscript in preparation). In parallel to the animal efficacy studies, safety and tolerability of rHuIL-12 was determined in normal healthy subjects as per the Animal Rule. A first in human (FIH) study was conducted to determine the safe and well-tolerated doses of rHuIL-12 via dose escalation (at doses ranging from 2 to 20 μg) which was followed by a phase 1b expansion study at the highest safe and well-tolerated dose from the FIH study of 12 μg (Gokhale et al, in preparation). The 12 μg unit human dose for a 70 kg adult can be converted to 171 ng/kg rhesus monkey dose using a weight based conversion and this dose is within the efficacious dose range as determined in our rhesus monkeys studies.

In summary, this randomized, placebo-controlled, blinded study has demonstrated that rHuIL-12 is an ideal frontline radiomitigator due its ability to increase survival and regenerate the hematopoietic system when administered 24 hours following radiation exposure as a single, low dose without supportive antibiotics, fluids or blood products. Translation of the efficacious and safe dose from an animal model to humans is a significant challenge for any drug development program under the Animal Rule. Thus, our finding that statistically significant increases in survival can be achieved over a ten-fold effective dose range of rHuIL-12 in the NHP model will provide a distinct advantage for optimal human dose selection.

TABLES

TABLE 1
Macroscopic Organ Hemorrhage Score and Organ
Infection Score Per Animal (Average ± SEM)

| | Total hemorrhage | GI tract hemorrhage | Total infection |
|---|---|---|---|
| Dose (ng/kg) | score^{a} | score^{b} | score^{c} |
| 0 | 8.4 ± 1.6 | 5.6 ± 1.1.4 | 12.8 ± 2.2 |
| 50 | 5.1 ± 0.63 | 3.2 ± 0.75 | 12.3 ± 3.4 |
| 100 | 6.6 ± 0.73 | 4.5 ± 0.90 | 10.6 ± 3.7 |
| 250 | 5.0 ± 1.0 | 3.1 ± 0.78 | 8.8 ± 3.1 |
| 500 | 5.8 ± 1.1 | 3.7 ± 0.70 | 8.2 ± 3.3 |

^{a}The following tissues were included in calculating the mean hemorrhage scores presented in this table: stomach, ileum, jejunum, duodenum, colon, cecum, rectum, heart, brain, kidneys, liver, lungs, urinary bladder). Hemorrhage score was defined as follows: 0 = absence of hemorrhage; 1 = minimal hemorrhage(s); 2 = slight hemorrhage(s); 3 = moderate hemorrhage(s); 4 = marked hemorrhage(s); and 5 = severe hemorrhage(s). In each animal scores for all organs were summed, then mean score for each treatment group was calculated.
^{b}The following tissues were included in calculating the GI tract hemorrhage score: stomach, ileum, jejunum, duodenum, colon, cecum
^{c}For infection score determination, organ samples were collected on necropsy and cultured (animals found dead excluded from the analysis), including brain, heart, kidney, liver, both lungs, and spleen. Bacterial growth was scored for each organ (from 0 to 4). In each animal scores for all organs were summed, then mean score for each treatment group was calculated.

### Example 12

Recombinant Interleukin-12 vs. Bone Marrow Transplant for Restoring Hematopoiesis after Lethal Irradiation in Mice and Evaluation of Potential Cellular Targets

In addition to its well-characterized immunomodulatory effects, interleukin-12 (IL-12) plays a role in restoring hematopoiesis in bone marrow damaged from irradiation. We have previously reported that recombinant murine IL-12 (rMuIL-12) alone, without supportive care, increased survival in lethally irradiated mice. Herein, we compared two schedules of administration of rMuIL-12 to bone marrow transplant (BMT) for rescue of mice exposed to lethal irradiation. Ten animals per group received one of the following: vehicle; 10 ng rMuIL-12 given 24 hours before total body irradiation (TBI); 10 ng rMuIL-12 given 24 hours before and 3 days after TBI; or donor bone marrow (1.1×106 cells) infused 2 hours after TBI. Results showed that survival rates in animals treated with rMuIL-12 were comparable to BMT. Additionally, hematopoietic recovery of neutrophils, red blood cells and platelets for both treatments were also comparable. In vivo experiments in mice showed that relative to vehicle, rMuIL-12 increased the expression of IL-12 receptor β2 subunit (IL-12β2) on bone marrow-derived lineage-negative (Lin−) cells, a subset of cells consisting of hematopoietic stem/progenitor cells. We further investigated the expression of IL-12Rβ2 on subsets of human hematopoietic stem/progenitor cells (HSCs). Human bone marrow-derived CD34+ HSCs expressed IL-12Rβ2 and IL-12Rβ2 co-expressed with other markers of human HSCs. These findings suggest that IL-12 function in hematopoietic recovery may be driven by interaction with IL-12Rβ2 expressing stem cells in bone marrow. These data motivate the study of adjuvant recombinant human IL-12 for enhancing hematopoietic recovery following bone marrow ablation in various clinical settings.

An ideal drug for mitigation of radiation-induced hematopoietic syndrome would mimic the effects of HSC transplantation (promotion of growth of multiple bone marrow cell lineages, reduction of morbidity, and improvement in survival rates) among victims of radiation exposure, would be safe when administered to healthy subjects, and could be administered at a single dose level to all victims. We previously proposed the pro-inflammatory cytokine interleukin 12 (IL-12) as such a candidate [1]. In addition to its well-established role in immunity [2], IL-12 also appears to play a fundamental role in preserving hematopoietic capabilities in damaged bone marrow. Early in vitro studies demonstrated that IL-12 together with either colony-stimulating factors (CSFs) [3] or IL-3 and steel factor (SF; also call stem cell factor [SCF]) [4,5,6] could induce proliferation of myeloid stem cells and progenitor cells [6], Lin−/Sca-1+ cells [3], lymphohematopoietic progenitors [5], or mixed, erythroid, and myeloid colonies [4]. In vivo studies by Neta et al. [7] demonstrated that IL-12 alone (1 μg/mouse) protected murine bone marrow from the effects of lethal irradiation, but increased radiosensitivity of the gastrointestinal system. Subsequently, Chen et al. [8] reported that an IL-12 dose of 100 ng/mouse was effective in protecting the bone marrow from lethal irradiation without increasing the radiosensitivity of the gastrointestinal system. We have recently reported that relative to placebo, doses of 10 to 40 ng of recombinant murine IL-12 (rMuIL-12) administered to mice 24 hours after lethal radiation increased survival, promoted hematopoiesis, as indicated by induction of IL-12 receptor β2 subunit-expression in bone marrow cells [1]. Further, in non-human primates exposed to lethal TBI, treatment with recombinant human IL-12 (rHuIL-12) resulted in a significant increase in survival relative to placebo-treated animals and attenuated the nadirs for leukocytes and platelets [1].

In the current study, we have compared the abilities of rMuIL-12 in inducing hematopoietic recovery and promoting survival to that of a conventional BMT in lethally irradiated mice. In addition, to further elucidate the potential cellular targets of IL-12, we evaluated the expression of the β2 subunit of the IL-12 receptor (IL-12Rβ2) in human bone marrow-derived FISCs.

Materials and Methods

Recombinant IL-12

rMuIL-12 was obtained from Peprotech (Catalog #210-12); Rocky Hill, N.J., USA) or SBH Sciences (LS #45[Q4]; Natick, Mass., USA The dose reported for each animal experiment is the dose determined by the ELISA (mouse IL-12 (p70) ELISA MAX™ Deluxe kit (Biolegend; San Diego, Calif.).

In Vivo Animal Studies

C57BL/6 mice were from Jackson Laboratories (Sacramento, Calif.) or Harlan Laboratories (Indianapolis, Ind.). At the time of study initiation, mice were 8 to 10 weeks old, weighed approximately 20 g, and had no signs of disease. All procedures were reviewed and approved by the BALI'S (Northridge Calif.) Laboratories Institutional Animal Care and Use Committee (accredited by the Association for the Assessment and Accreditation of Laboratory Animal Care and the American Association of Laboratory Animal Care) and were in accordance with guidelines of the National Institutes of Health [9].

Comparison of Effect of rMuIL-12 and BMT on Survival in Mice Exposed to Lethal Radiation

The effects of rMuIL-12 and BMT on survival were compared in 40 female mice exposed to lethal TBI. Ten mice each were assigned to one of the following intravenous treatments: vehicle (phosphate-buffered saline [PBS]; 100 μL) administered 24 hours before TBI; 10 ng rMuIL-12 administered 24 hours before TBI (hereafter referred to as single-dose rMuIL-12); 10 ng rMuIL-12 administered 24 hours before and 3 days after TBI (hereafter referred to as repeat-dose rMuIL-12); or donor bone marrow (1.1×106 cells) administered 2 hours after TBI. Donor marrow cells for transplantation were isolated from normal female C57BL/6 mice by flushing femurs and tibias with PBS using a 25-gauge ⅝-inch needle. Cells were filtered through a 40 μm cell strainer, washed with RPMI media containing 10% fetal bovine serum, and cryopreserved in liquid nitrogen. On Day 0, mice were exposed to TBI at a dose of 8.2 Gy (targeted LD90 [1]) using a Gammacell® 40 (137Cs) (Theratronics, Ontario, Canada). Mice were centered in the irradiator using a “pie-box” for radiation distribution. Following TBI, mice had access to sterilized food and acidified water ad libitum and were monitored for survival until Day 35. Blood was drawn on Days 21, 28 and 35 from 5 animals in each group. Blood cell counts were determined by Hemavet® 850 (Drew Scientific Inc.; Waterbury, Conn.).

Flow Cytometry Analysis of In Vivo Effect of rMuIL-12 on IL-12Rβ2 Receptor Beta-2 Subunit (IL-12Rβ2) Expression on Lin− Cells in Healthy Female C57BL/6 Mice

Mice (6 per treatment group) received rMuIL-12 (10 ng) via tail vein injection or vehicle (PBS). Bone marrow was harvested 21 hours after rMuIL12 treatment. Marrow flushed from femurs and tibias was diluted with MACS buffer (Miltenyi Biotec; Auburn, Calif.) and filtered through a 70 μm cell strainer (VWR; San Francisco, Calif.). Cells were washed and incubated with biotin-conjugated monoclonal antibodies against the following murine lineage markers (Lin+): CD3e, CD4, CD5, CD8b, CD8a, B220, CD11b, Grl and Ter (80) (Miltenyi Biotec; Auburn, Calif.). Cells were incubated with anti-biotin-labeled magnetic beads and passed through a MACS column placed in the magnetic field of a QuadroMACS separator (Miltenyi Biotec; Auburn, Calif.). Unlabeled Lin− cells were collected as effluent and incubated with anti-mouse IL-12Rβ2 antibody (HAM 10B9, BD Biosciences; San Jose, Calif.) for 30 minutes at room temperature. Cells were analyzed on a MoFlow cell cytometer (Beckman Coulter; Indianapolis, Ind.) with unstained and isotype controls. The experiment was repeated with bone marrow harvested at 25 hours after rMuIL-12 and Lin− cells analyzed for IL-12R□2 expression as mentioned earlier.

Immunocytochemical Detection of IL-12Rβ2 in Human Bone Marrow CD34+ Cells

Human bone marrow-derived CD34+ cells were obtained commercially from All Cells (Emeryville, Calif.). Cells were seeded on 5 μg/mL fibronectin slides and fixed in cold methanol, 10 minutes 20° C. Fixed cells were treated with 0.3% H202 for 30 minutes to eliminate endogenous peroxidase staining and then 20 minutes with Background Sniper (Biocare Medical; Concord, Calif.) to eliminate non-specific staining. Treated cells were labeled with a rabbit polyclonal antibody against human IL-12Rβ2 (Sigma; St Louis, Mo.) followed by incubation with anti-rabbit IgG coupled to horseradish peroxidase (ImmPRESS reagent; Vector Laboratories; Burlingame, Calif.). Slides were incubated with ImmPACT AEC peroxidase substrate (Vector Laboratories; Burlingame, Calif.) for 30 minutes and were counterstained in CAT Hematoxylin (Biocare Medical, LLC; Concord, Calif.). The negative control included cells labeled with anti-rabbit IgG coupled to horseradish peroxidase without primary antibody. Images were acquired using an Olympus BX41 compound microscope (Olympus, America, Inc; Center Valley, Calif.) and Infinity Analyze Software v5.0. Analysis was done in triplicate with CD34+ cells from three individual donors.

Flow Cytometry Analysis of Expression of IL-12Rβ2 on Human Bone Marrow-Derived Stem/Progenitor Cells

Human bone marrow-derived CD34+ cells were obtained commercially, as described above. CD34+ cells were washed and incubated for 30 minutes at room temperature with a LIVE/DEAD Fixable dead cell stain (Invitrogen; Grand Island, N.Y.) which is a viability marker. Cells were washed and incubated with antibodies to each of the following hematopoietic stem cell markers: fluorescein isothiocyanate (FITC)-conjugated cKit/CD117 (clone # YB5.B8; BD Biosciences; San Jose, Calif.), phycoerythrin (PE)-conjugated CD133 (clone #293C3; BD Biosciences), PE-Flt-3/CD135 (clone #4G8; BD Biosciences), PE-Slam/CD150 (clone # Al2; BD Biosciences), PE-VegfR2/KDR (clone #89106; R&D systems; Minneapolis, Minn.), PE-CDCP1/CD318 (clone #309121; R&D systems), FITC-CD34 (clone # AC136; Miltenyi Biotec; Auburn, Calif.). Isotype matched control for each antibody listed above was analyzed in parallel to determine background fluorescence. Cells were washed and fixed and permeabilized using BD Cytofix/Cytoperm fixation/permeabilization kit (BD Biosciences; San Hose, Calif.). Intracellular IL-12R□2 was determined using an allophycocyanin-conjugated (APC) antibody against human IL-12R□2 (Clone #305719; R&D systems; Minneapolis, Minn.). Isotype matched control for IL-12R□2 antibody was analyzed in parallel. Cells were analyzed using a Special Order Research Product (SORP) BD™ LSRII digital flow cytometer (BD BioScience; San Jose, Calif.; License # H47300022) equipped with three high-powered, solid state lasers (wavelengths of 488 nm, 635 nm and near-UV) located at the Broad CIRM Center at University of Southern California-Health Science Campus. This cytometer is specialized for acquisition of data with up to 10 fluorescent parameters. Data were analyzed using Flow.Jo version 9.5.2 (TreeStar, Ashland, USA). Gating strategy was as follows: plot of forward versus side scatter to select CD34 cells, side scatter versus live/dead cell stain for dead cell exclusion, height versus area scatter to exclude doublets (data not shown) and IL-12R□2 versus stem cell marker. Quadrant gates were set based on isotype matched antibody controls. Analysis was done in triplicate with CD34+ cells from three individual donors.

Statistical Analyses

Survival curves for all treatment groups were statistically compared using Cox-Mantel test. Blood cell recovery for all groups was analyzed by Student T-test. Mean with standard deviation was reported when applicable.

Results

rMuIL-12 Increases Survival and Promotes Blood Cell Recovery as Effectively as BMT in Healthy Mice Exposed to Lethal Radiation

BMT and either single or repeat doses of rMuIL-12 showed similar abilities to increase survival compared with vehicle in C57BL/6 mice exposed to lethal TBI (Figure I), with Day 35 overall survival (OS) of 0% in the vehicle group, 70% in the BMT group, 60% in the single-dose rMuIL-12 group, and 90% in the repeat-dose rMuIL-12 group. OS with rMuIL-12 was comparable to that with BMT. OS in each of the rMuIL-12 groups and BMT groups was significantly higher than the vehicle group using Cox-Mantel test (P<0.005 for each comparison). At Day 21, neutrophil levels in BMT group were significantly higher than that of single-dose rMuIL-12 (p<0.05) (FIG. 2A). Neutrophils levels in repeat-dose rMuIL-12 group were not statistically different from BMT group. At Day 28 and Day 35, neutrophil levels in all treatment groups were comparable statistically. RBC levels in the BMT group were significantly higher than both rMuIL-12 groups at Day 21 (p<0.005) (FIG. 2B). RBC levels for all treatment groups were comparable on Day 28 and Day 35. Platelet levels were significantly higher for the repeat-dose rMuIL-12 group compared to single-dose rMuIL-12 (p<0.005) and BMT at Day 21 (p<0.05) (FIG. 2C). At Day 28 and Day 35, platelet levels for all treatment groups were comparable statistically. Error bars represent Mean±SD. Overall, recovery of neutrophils, platelets, and red blood cells (RBCs) by Day 35 with rMuIL-12 was comparable to BMT.

In Vivo Treatment with rMuIL-12 Increases the Expression of IL-12 Receptor, Beta-2 Subunit (IL-12Rbeta2) on Bone Marrow-Derived Lineage Depleted (Lin−) Cells

To elucidate expression of IL-12Rbeta 2 in murine bone marrow, we treated healthy female C57BL/6 mice with a single, intravenous, 10-ng dose of rMuIL-12 (n=6) or vehicle (n=6) via tail vein injection and examined expression of IL12Rbeta2 in Lin− cells isolated from the bone marrow. Results from two experiments showed that rMuIL-12 induced a 3.5-fold to 5.4-fold increase, (mean (±SD)−4.45±1.3) in the expression of IL-12Rb2 on Lin− cells (Table 1) 21 to 25 hours post-treatment. Lin− cells represent a cell population consisting of hematopoietic stem/progenitor cells and depleted of mature hematopoietic cells and committed precursor progenitors. Our data shows that IL-12 increases the IL-12Rbeta2 expression on Lin− cells in murine bone marrow. This data with the hematopoietic recovery function of IL-12 suggests that IL-12Rbeta2 may be expressed on HSCs.

Human Bone Marrow CD34+ Cells Express IL-12R β 2

We next elucidated IL-12Rbeta2 expression on human bone marrow CD34 expressing HSCs. First, using immunocytochemical staining, we demonstrated the presence of IL-12R β 2 on commercially available human bone marrow CD34+ cells (FIG. 3A). Second, we quantified the expression of IL-12R β 2 on human CD34+ cells by flow cytometry. Among the CD34+ cells from three individual donors tested, the mean (±SD) percentage that expressed IL-12R β 2 was 89±2 (FIG. 3B).

Human Bone Marrow CD34+ Cells Co-Express IL-12Rβ2 and Other Known Hematopoietic Stem/Progenitor Cell (USC) Markers

We next sought to examine IL-12Rβ2 co-expression with HSC markers CD117, CDI33, CD135, and CD318 to further support our observation of IL-12Rβ2 expression on CD34+ cells. These markers were chosen because they have been shown to represent HSC populations with engraftment potential [18, 23, 24, 25, 26, 27]. IL-12Rβ2 co-expressed with CD 117, CD133, CD135 and CD318 on human bone marrow CD34+ cells. The mean percentages of CD34+ cells that co-expressed IL-12β2 and CD117, CD133, CD135, and CD318 were 70±11%, 63±7%, 57±4%, and 61±9%, respectively (Table 2 and FIGS. 4A through 4D). Notably, the majority of CD 117, CD133, CD135, and CD318 expressing CD34 sub-populations showed IL-12Rβ2 expression. These results suggest a potential role for IL-12Rβ2 in IL-12 mediated hematopoietic recovery via HSC populations in the bone marrow.

Discussion

The current study compared the in vivo hematopoietic activity and survival benefit resulting from treatment with rMuIL-12 or BMT in lethally irradiated mice. Results demonstrated that irradiated mice treated with BMT or either one or two 10 ng doses of rMuIL-12 had significantly increased survival compared with vehicle control. Survival curves among the three active treatment groups were not significantly different from each other. Comparable recovery of neutrophils, RBCs, and platelets were observed in all three active treatment groups by Day 35. Platelet levels in repeat-dose rMuIL-12 were significantly higher at earlier timepoint Day 21 compared to BMT supporting the role of IL-12/IL-12R in platelet recovery. These data confirm and extend previous data from our laboratory [1] and from other investigators [7,8]. We also have observed similar effects of rMuIL-12 on blood cell recovery in sublethally irradiated mice bearing Lewis lung and EL4 lymphoma tumors [10].

The ability of rMuIL-12 to promote survival and blood cell recovery in irradiated murine bone marrow suggests that functional IL-12 receptors (IL-12R) are present on bone marrow cells. The IL-12 receptor is a heterodimeric transmembrane protein composed of a β1 subunit (IL-12R β1) that is common to IL-23 receptor complex [11] and an IL-12 receptor-specific beta2 subunit (IL-12Rbeta2) [2]. Using immunohistochemical analysis, we previously reported that IL-12Rbeta2 was expressed on bone marrow cells in mice [1]. Because the murine bone marrow immunohistochemistry data suggested that IL-12 treatment promoted the proliferation and differentiation of hematopoietic bone marrow cells, in the current paper we examined whether rMuIL-12 treatment in normal mice would affect IL-12Rbeta2 expression in the Lin− population, which represents a population of hematopoietic stem/progenitor cells. Results showed that relative to control mice, those treated with rMuIL-12 showed a 3- to 5-fold increase in the expression of IL-12Rbeta2 on bone marrow-derived Lin− cells.

We further sought to determine if IL-12Rbeta2 was expressed in human bone marrow. Immunocytochemistry showed the presence of IL-12Rbeta2 in human bone marrow-derived CD34+ cells, and flow cytometry analysis revealed that CD34+ cells express 12Rbeta2. The current study is the first to show that IL-12Rbeta2 is expressed on human CD34+ cells, which represents a mixed population of hematopoietic stem and progenitor cells in the bone marrow. We identified IL-12R β 2 expression in 87-90% of normal human marrow-derived CD34+ cells, which represent a population of cells that possess long term reconstitution potential and are used for bone marrow transplantations in the clinical setting [12, 13, 14]. Further, subpopulations of CD34 cells expressing the markers CD117 (c-Kit), CD133, CD135 (Flt3), and CD318 (CDCP1), all of which have been used to identify HSC populations, co-expressed IL-12R 2. CD 117 is the receptor for SCF, which has been shown to function as a hematopoietic growth factor involved in expansion of HSCs [15] and has been implicated in the mobilization of HSCs [5,15]. Interestingly, IL-12 and SCF have been shown to synergistically support proliferation of lymphohematopoietic progenitors [5]. CD133 has been reported to be expressed in neural and hematopoietic stem/progenitor cells [16], and CD34+/CD133+ cells can engraft in NOD/SCID mice [17]. CD135, a member of the receptor tyrosine kinase family, plays a critical role in maintenance of hematopoietic homeostasis, and has been implicated in expansion of hematopoietic progenitor cells [18]. Flt3-ligand has been shown to synergize with IL-12 and other cytokines and growth factors in expansion of bone marrow HSCs [19]. CD318+ cells have been shown to possess multilineage differentiation potential when engrafted in NOD/SCID mice [20]. Taken together, the IL-12Rβ2 expression data from murine and human bone marrow suggest that IL-12Rβ2 expressing HSCs could be potential targets of the hematopoietic activity of IL-12.

The apparent ability of IL-12 to stimulate hematopoiesis and promote survival of animals with lethally damaged bone marrow suggests that IL-12 may be a useful therapeutic in situations of damaged bone marrow, whether induced clinically, as part of a pre-transplant regimen, or as a result of a radiation accident or disaster. HemaMax™ (Neumedicines Inc.; Pasadena, Calif.), a recombinant human interleukin-12 preparation (rHuIL-12), is being developed for treatment of the hematopoietic syndrome of the acute radiation syndrome (HSARS) under the FDA Animal Rule. We have recently reported that a single, low dose (100 ng/kg or 250 ng/kg) of HemaMax™ administered 24 hours after exposure to lethal radiation can reconstitute the bone marrow and increase survival in nonhuman primates (NHP) [1]. In addition, our data showed that rHuIL-12 attenuated the nadir for leukocytes and platelets in lethally irradiated NHPs [1].

With respect to BMT in the oncology setting, the hematopoietic properties of IL-12 may be applicable as an adjuvant therapeutic. Improving techniques in bone marrow stem cell mobilization, developing apheresis approaches for their collection, and using new strategies for reducing toxicity have improved the outcome of HSC transplantation and expanded its therapeutic value to a wider population of cancer patients [21, 22, 23]. Nevertheless, the success of HSC transplantation is largely limited by number of cells required for long-term reconstitution of the hematopoietic system. Conventional transplantations employ the use of CD34+ cells, which are isolated by mobilization with growth factors, such as G-CSF, yet the requirement for multiple rounds of G-CSF treatment to isolate sufficient quantities of CD34+ cells may be immunosuppressive [24,25]. Myeloid and erythroid growth factors are used to treat post-transplant neutropenia and anemia [26], but do not affect platelet regeneration. Thus, to overcome post-transplant thrombocytopenia, which can occur in as many as 17% of patients with cancer and significantly increases risk of death [27], patients must rely on early post-transplant platelet transfusions [28]. The increased level of platelets observed after IL-12 treatment in mice exposed to lethal radiation as observed in the current study, as well as in our previous studies [1,10], suggests that IL-12 may be beneficial in promoting post-transplant platelet recovery in humans.

Although numerous clinical trials have evaluated IL-12 as an anti-tumor agent in cancer patients, the agent has not advanced as a therapeutic due to its modest clinical activity and significant toxicity when administered as repeated, high dose (300 ng/kg to 600 ng/kg) regimens (5 doses per week) [29, 30, 31, 32, 33, 34, 35, 36, 37]. By contrast, IL-12 doses of 30 to 250 ng/kg given as a single dose approximately 2 months after peripheral blood stem cell transplantation and then for 5 consecutive days 2 weeks later, showed acceptable tolerability in patients with high-risk hematological malignancies [37]. As noted above, rHuIL-12, like its murine counterpart, exerted its hematopoietic-promoting activity at doses of 100 to 250 ng/kg when administered only once or twice to rhesus monkeys exposed to lethal TBI [1]. These doses are equivalent to a human dose of less than 100 ng/kg. In our current Phase 1 human safety studies, rHuIL-12 has been well tolerated and no safety issues have been identified (Neumedicines, Inc., unpublished data). These findings suggest that the rHuIL-12 dose required for hematopoietic regeneration will be substantially lower than the IL-12 doses previously used in cancer patients and should result in a more favorable safety profile for rHuIL-12 in the settings of both ARS and HSC transplantation for cancer.

### CONCLUSIONS

We have demonstrated that rMuIL-12 is equivalent to BMT for promoting hematopoietic recovery and survival in mice exposed to lethal radiation, and that rMuIL-12 treatment increased levels of IL-12Rβ2 expression in Linβ cells isolated from normal mice. Further, CD34+ cells from normal human bone marrow were found to express IL-12Rβ2. IL-12Rβ2 also co-expressed with other known HSC markers was observed. These findings begin to bring to light the importance of the IL-12/IL-12R system to stem cell biology and regenerative medicine and suggest that this pathway represents a therapeutic target for increasing remission and survival in patients undergoing HSC transplantation or suffering the effects of lethal radiation exposure in a nuclear disaster. Thus, clinical studies are warranted to evaluate the safety and efficacy of adjuvant rHuIL-12 therapy in these settings.

TABLE 1
Effect of rMuIl-12 on Expression of IL-12Rβ2 in
Lin⁻ Cells Isolated from Murine Bone Marrow

Percentage of Lin− cells expressing IL12-2β2

| | Vehicle | 10 ng rMuIL-12 | |
|---|---|---|---|
| Experiment | (N = 6)* | (N = 6)* | Fold increase |
| 1 | 0.6 | 2.1 | 3.5 |
| 2 | 0.5 | 2.7 | 5.4 |

Notes:
Flow cytometry analyses were conducted with bone marrow Lin cells collected 21 hours or 25 hours after treatment with rMuIL-12 in Experiments 1 and 2, respectively.
*Data represent the mean of 6 mice per treatment group per experiment.

TABLE 2
Percentage of Human Bone Marrow-CD34⁺ Cells that Co-express
IL-12Rβ2 and Other Hematopoietic Stem Cell Markers

| | Percentage of CD34+ cells |
|---|---|
| | Expressing IL-12Rβ2 and |
| | Indicated Marker |
| Cell Population/CD Marker | Mean + SD |
| CD34⁺ | |
| IL- 12Rβ2+/CD 117(c-kit)⁺ | 70 ± 11 |
| IL- 12Rβ2+/CD 133+ | 63 ± 7 |
| IL- 12Rβ2+/CD135(Flt3)⁺ | 57 ± 4 |
| IL- 12Rβ2+/CD318(CDCP1)⁺ | 6 1 ± 9 |

Notes:
Data represent summary statistics based on cells from three different healthy biological donors

While this invention has been disclosed with reference to specific embodiments, it is apparent that other embodiments and variations of this invention can be devised by those skilled in the art without departing from the true spirit and scope of the invention. The appended claims include all such embodiments and equivalent variations.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

### REFERENCES

1. Waselenko J K, MacVittie T J, Blakely W F, et al. Medical management of the acute radiation syndrome: recommendations of the Strategic National Stockpile Radiation Working Group. Ann intern Med. 2004; 140:1037-1051.
2. DiCarlo A L, Maher C, Hick J L, et al. Radiation injury after a nuclear detonation: medical consequences and the need for scarce resources allocation. Disaster Med Public Health Prep. 2011; 5 Suppl 1:S32-44.
3. Dainiak N, Waselenko J K, Armitage J O, MacVittie T J, Farese A M. The hematologist and radiation casualties. Hematology Am Soc Hematol Educ Program. 2003:473-496.
4. Bellone G, Trinchieri G. Dual stimulatory and inhibitory effect of NK cell stimulatory factor/IL-12 on human hematopoiesis. J Immunol. 1994; 153:930-937.
5. Dybedal I, Larsen S, Jacobsen S E. IL-12 directly enhances in vitro murine erythropoiesis in combination with IL-4 and stem cell factor. J Immunol. 1995; 154:4950-4955.
6. Jacobsen S E, Veiby O P, Smeland E B. Cytotoxic lymphocyte maturation factor (interleukin 12) is a synergistic growth factor for hematopoietic stem cells. J Exp Med. 1993; 178:413-418.
7. Trinchieri G. Interleukin-12: a cytokine at the interface of inflammation and immunity. Adv Immunol. 1998; 70:83-243.
8. Basile L A, Ellefson D, Gluzman-Poltorak Z, et al. HemaMax, a Recombinant Human Interleukin-12, Is a Potent Mitigator of Acute Radiation Injury in Mice and Non-Human Primates. PLoS One. 2012; 7:e30434.
9. Farese A M, Cohen M V, Katz B P, et al. Filgrastim improves survival in lethally irradiated nonhuman primates. Radiat Res. 2013; 179:89-100.
10. Friesecke I, Beyrer K, Fliedner™. How to cope with radiation accidents: the medical management. Br J Radiol. 2001; 74:121-122.
11. Basile L A, Gallaher T K, Shibata D, Miller J D, Douer D. Multilineage hematopoietic recovery with concomitant antitumor effects using low dose Interleukin-12 in myelosuppressed tumor-bearing mice. J Transl Med. 2008; 6:26.
12. Park H R, Jo S K, Paik S G. Factors effecting the Th2-like immune response after gamma-irradiation: low production of IL-12 heterodimer in antigen-presenting cells and small expression of the IL-12 receptor in T cells. Int J Radiat Biol. 2005; 81:221-231.
13. Park H R, Jo S K, Paik S G. The NK1.1+T cells alive in irradiated mice play an important role in a Th1/Th2 balance. Int J Radiat Biol. 2006; 82:161-170.
14. Colombo M P, Trinchieri G. Interleukin-12 in anti-tumor immunity and immunotherapy. Cytokine Growth Factor Rev. 2002; 13:155-168.
15. Williams J P, Brown S L, Georges G E, et al. Animal models for medical countermeasures to radiation exposure. Radiat Res. 2010; 173:557-578.
16. Gerosa F, Baldani-Guerra B. Nisii C, Marchesini V, Carra G, Trinchieri G. Reciprocal activating interaction between natural killer cells and dendritic cells. J Exp Med. 2002; 195:327-333.
17. Trinchieri G. Interleukin-12 and the regulation of innate resistance and adaptive immunity. Nat Rev Immunol. 2003; 3:133-146.
18. Azoulay, E., Attalah, H., Harf, A., Schlemmer, B., and Delclaux C. 2001. Granulocyte colony-stimulating factor or neutrophil-induced pulmonary toxicity: myth or reality? Chest 120:1695-1701.
19. Basile L A, Ellefson D, Gluzman-Poltorak Z et al. (2012) HemaMax, a Recombinant Human Interleukin-12, Is a Potent Mitigator of Acute Radiation Injury in Mice and Non-Human Primates. PLoS One 7: e30434.
20. Gately M K, Renzetti L M, Magram J, Stern A S, Adorini L, et al. (1998) The interleukin-12/interleukin-12-receptor system: role in normal and pathologic immune responses. Annu Rev Immunol 16: 495-521.
21. Jacobsen S E, Veiby O P, Smel and EB (1993) Cytotoxic lymphocyte maturation factor (interleukin 12) is a synergistic growth factor for hematopoietic stem cells. J Exp Med 178: 413-418.
22. Bellone G, Trinchieri G (1994) Dual stimulatory and inhibitory effect of NK cell stimulatory factor/IL-12 on human hematopoiesis. J Immunol 153: 930-937.
23. Hirayama F, Katayama N, Neben S, Donaldson D, Nickbarg E B, et al. (1994) Synergistic interaction between interleukin-12 and steel factor in support of proliferation of murine lymphoheinatopoietic progenitors in culture. Blood 83: 92-98.
24. Ploemacher R E, van Soest P L, Boudewijn A, Neben S (1993) Interleukin-12 enhances interleukin-3 dependent multilineage hematopoietic colony formation stimulated by interleukin-11 or steel factor. Leukemia 7: 1374-1380.
25. Neta R, Stiefel S M, Finkelman F, Herrmann S, Ali N (1994) IL-12 protects bone marrow from and sensitizes intestinal tract to ionizing radiation. J Immunol 153: 4230-4237.
26. Chen T, Burke K A, Zhan Y, Wang X, Shibata D, et al. (2007) IL-12 facilitates both the recovery of endogenous hematopoiesis and the engraftment of stem cells after ionizing radiation. Exp Hematol 35: 203-213.
27. (1996) US Food and Drug Administration, Guide for the Care and Use of Laboratory Animals. US National Institutes of Health.
28. Basile L A, Gallaher T K, Shibata D, Miller J D, Douer D (2008) Multilineage hematopoietic recovery with concomitant antitumor effects using low dose Interleukin-12 in myelosuppressed tumor-bearing mice. J Transl Med 6: 26.
29. Trinchieri G, Pflanz S, Kastelein R A (2003) The IL-12 family of heterodimeric cytokines: new players in the regulation of T cell responses. Immunity 19: 641-644.
30. Civin C I, Almeida-Porada G, Lee M J, Olweus J, Terstappen L W, et al. (1996) Sustained, retransplantable, multilineage engraftment of highly purified adult human bone marrow stem cells in vivo. Blood 88: 4102-4109.
31. Terstappen L W, Huang S, Safford M, Lansdorp P M, Loken M R (1991) Sequential generations of hematopoietic colonies derived from single nonlineage-committed CD34+CD38− progenitor cells. Blood 77: 1218-1227.
32. Moog R (2008) Management strategies for poor peripheral blood stem cell mobilization. Transfus Apher Sci 38: 229-236.
33. Bodine D M, Seidel N E, Zsebo K M, Orlic D (1993) In vivo administration of stem cell factor to mice increases the absolute number of pluripotent hematopoietic stem cells. Blood 82: 445-455.
34. Florek M, Haase M, Marzeseo A M, Freund D, Ehninger G, et al. (2005) Prominin-1/CD133, a neural and hematopoietic stem cell marker, is expressed in adult human differentiated cells and certain types of kidney cancer. Cell Tissue Res 319: 15-26.
35. Drake A C, Khoury M, Leskov I, Iliopoulou B P, Fragoso M, et al. (2011) Human CD34+ CD133+ hematopoietic stem cells cultured with growth factors including Angptl5 efficiently engraft adult NOD-SOD Il2rgamma−/− (NSG) mice. PLoS One 6: e18382.
36. Kikushige Y, Yoshimoto G, Miyamoto T, lino T, Mori Y, et al. (2008) Human Flt3 is expressed at the hematopoietic stem cell and the granulocyte/macrophage progenitor stages to maintain cell survival. J Immunol 180: 7358-7367.
37. Jacobsen S E W, Okkenhaug C, Myklebust J, Veiby O P and Lyman S D (1995) The Flt3 ligand potently and directly stimulates the growth and expansion of primitive murine bone marrow progenitor cells in vitro: Synergistic interactions with Interleukin (IL) 11, IL-12 and other hematopoietic growth factors. J Exp Med 81: 1357-1363
38. Takeda H, Fujimori Y, Kai S, Ogawa H, Nakano T (2010) CD318/CUB-domain-containing protein 1 expression on cord blood hematopoietic progenitors. Exp Ther Med 1: 497-501.
39. Bakanay S M, Demirer T (2011) Novel agents and approaches for stem cell mobilization in normal donors and patients. Bone Marrow Transplant.
40. Gyurkocza B, Rezvani A, Storb R F (2010) Allogeneic hematopoietic cell transplantation: the state of the art. Expert Rev Hematol 3: 285-299.
41. Hill-Kayser C E, Plastaras J P, Tochner Z, Glatstein E (2011) TBI during BM and SCT: review of the past, discussion of the present and consideration of future directions. Bone Marrow Transplant 46: 475-484.
42. Anderlini P, Przepiorka D, Seong D, Champlin R, Korbling M (1996) Transient neutropenia in normal donors after G-CSF mobilization and stem cell apheresis. Br J Haematol 94: 155-158.
43. Stroncek D F, Clay M E, Smith J, Ilstrup S, Oldham F, et al. (1996) Changes in blood counts after the administration of granulocyte-colony-stimulating factor and the collection of peripheral blood stem cells from healthy donors. Transfusion 36: 596-600.
44. Held T K, Gundert-Remy U (2010) Pharmacodynamic effects of haematopoietic cytokines: the view of a clinical oncologist. Basic Clin Pharmacol Toxicol 106: 210-214.
45. Ninan M J, Flowers C R, Roback J D, Arellano M L, Waller E K (2007) Posttransplant thrombopoiesis predicts survival in patients undergoing autologous hematopoietic progenitor cell transplantation. Biol Blood Marrow Transplant 13: 895-904.
46. Wandt H, Ehninger G, Gallmeier W M (2001) New strategies for prophylactic platelet transfusion in patients with hematologic diseases. Oncologist 6: 446-450.
47. Atkins M B, Robertson M J, Gordon M, Lotze M T, DeCoste M, et al. (1997) Phase I evaluation of intravenous recombinant human interleukin 12 in patients with advanced malignancies. Clin Cancer Res 3: 409-417.
48. Bajetta E, Del Vecchio M, Mortarini R, Nadeau R, Rakhit A, et al. (1998) Pilot study of subcutaneous recombinant human interleukin 12 in metastatic melanoma. Clin Cancer Res 4: 75-85.
49. Gollob J A, Veenstra K G, Parker R A, Mier J W, McDermott D F, et al. (2003) Phase I trial of concurrent twice-weekly recombinant human interleukin-12 plus low-dose IL-2 in patients with melanoma or renal cell carcinoma. J Clin Oncol 21: 2564-2573.
50. Lenzi R, Rosenblum M, Verschraegen C, Kudelka A P, Kavanagh J J, et al. (2002) Phase I study of intraperitoneal recombinant human interleukin 12 in patients with Mullerian carcinoma, gastrointestinal primary malignancies, and mesothelioma. Clin Cancer Res 8: 3686-3695.
51. Little R F, Aleman K, Kumar P, Wyvill K M, Pluda J M, et al. (2007) Phase 2 study of pegylated liposomal doxorubicin in combination with interleukin-12 for AIDS-related Kaposi sarcoma. Blood 110: 4165-4171.
52. Motzer R J, Rakhit A, Schwartz L H, Olencki T, Malone™, et al. (1998) Phase I trial of subcutaneous recombinant human interleukin-12 in patients with advanced renal cell carcinoma. Clin Cancer Res 4: 1183-1191.
53. Ohno R, Yamaguchi Y, Toge T, Kinouchi T, Kotake T, et al. (2000) A dose-escalation and pharmacokinetic study of subcutaneously administered recombinant human interleukin 12 and its biological effects in Japanese patients with advanced malignancies. Clin Cancer Res 6: 2661-2669.
54. Portielje J E, Kruit W H, Schuler M, Beck J, Lamers C H, et al. (1999) Phase 1 study of subcutaneously administered recombinant human interleukin 12 in patients with advanced renal cell cancer, Clin Cancer Res 5: 3983-3989.
55. Robertson M J, Pelloso D, Abonour R, Hromas R A, Nelson R P, Jr., et al. (2002) Interleukin 12 immunotherapy after autologous stem cell transplantation for hematological malignancies. Clin Cancer Res 8: 3383-3393.

1. A method of restoring hematopoiesis and reducing infectious complications comprising administering one or more effective dose(s) of IL-12 following myeloablative therapy.

2. A method of restoring hematopoiesis and reducing infectious complications comprising administering one or more effective dose(s) of IL-12 prior to myeloablative therapy.

3. (canceled)

4. The method of claim 1, where radiation therapy is used in the myeloablative therapy.

5. The method of claim 1, where chemotherapy is used in the myeloablative therapy.

6. The method of claim 1, wherein hematopoiesis is restored via an increased number of progenitor cells in the bone marrow.

7-12. (canceled)

13. A method for restoring hematopoiesis comprising administering one or more effective dose(s) of IL-12 either before, after, or before and after myeloablative therapy, wherein hematopoiesis is restored via activation of the IL-12 receptor on hematopoietic cells in the bone marrow.

14. The method of claim 13, wherein the hematopoietic cells comprise niche cells and stem cells.

15. The method of claim 14, wherein the niche cells comprise osteoblasts.

16. The method of claim 13, where hematopoiesis is restored following activation of the IL-12 receptor on megakaryocytes.

17. (canceled)

18. The method of claim 13, wherein hematopoiesis is restored following activation of the IL-12 receptor on osteoblastic cells, megakaryocyte cells, and hematopoietic stem cells in the bone marrow.

19. (canceled)

20. The method of claim 1, wherein the myeloablative therapy is followed by an autologous transplant.

21. The method of claim 1, wherein the myeloablative therapy is followed by an allogenic transplant.

22. The method of claim 1, wherein mobilization and collection of hematopoietic stem cells is done prior to myeloablative therapy.

23. The method of claim 22, wherein mobilization and collection of hematopoietic stem cells yields a low count of CD34+ cells.

24. (canceled)

25. The method of claim 1, wherein the myeloablative therapy is given to treat a hematopoietic malignancy selected from the group consisting of chronic myeloid leukemia, chronic lymphocytic leukemia, mantle cell lymphoma, low-grade non-Hodgkin's lymphoma, acute myeloid leukemia, intermediate grade lymphoma, multiple myeloma, myelodysplastic syndrome and Hodgkin's disease.

26. (canceled)

27. The method of claim 1, wherein the IL-12 is a recombinant human IL-12.

28. The method of claim 1, wherein the myeloablative method is a combination of radiation therapy and chemotherapy.

29. The method of claim 1, wherein the method comprises a non-myeloablative method.

30. The method of claim 29, wherein the non-myeloablative method comprises mini-transplant.

31. The method of claim 29, wherein the non-myeloablative method comprises reduced intensity conditioning (RIC).
